(19) United States
(12) Patent Application Publication
(10) Pub. No.: US 2023/0032478 A1
(43) Pub. Date: Feb. 2, 2023

(54) Methods for Improved Regeneration of Plants Using Growth-Regulating Factor (GRF), GRF-Interacting Factor (GIF), or Chimeric GRF-GIF

(71) Applicants: THE REGENTS OF THE UNIVERSITY OF CALIFORNIA, Oakland, CA (US); CONSEJO NACIONAL DE INVESTIGACIONES CIENTÍFICAS Y TÉCNICAS, Ciudad Autonoma de Buenos Aires (AR)

(72) Inventors: JORGE DUBCOVSKY, Oakland, CA (US); JUAN MANUEL DEBERNARDI, Oakland, CA (US); DAVID TRICOLI, Oakland, CA (US); JAVIER PALATNIK, Ciudad Autonoma de Buenos Aires (AR)

(21) Appl. No.: 17/597,511

(22) PCT Filed: Jul. 8, 2020
(86) PCT No.: PCT/US20/41135

§ 371 Date: Jan. 10, 2022

Related U.S. Application Data
(60) Provisional application No. 62/873,123, filed on Jul. 11, 2019.

(51) Int. Cl. C12N 15/82; C07K 14/475

(52) U.S. Cl. CPC C12N 15/8262; C07K 14/475; C12N 15/8295

(57) ABSTRACT

Disclosed are methods of producing plants with an improved regeneration efficiency using Growth-Regulating Factor (GRF), GRF-Interacting Factor (GIF), or chimeric GRF-GIF genes and proteins. The disclosure also provides plants with an improved regeneration efficiency that are produced by the disclosed methods, methods of reducing the use of exogenous cytokinins in the regeneration of plants, and methods of improving the regeneration efficiency of plants.

### REFERENCE TO RELATED APPLICATION

This application claims priority to PCT/US2020/041135, filed Jul. 8, 2020, which claims priority to provisional application U.S. Ser. No. 62/873,123, filed Jul. 11, 2019, all of which are incorporated herein by reference in their entirety.

### STATEMENT REGARDING FEDERALLY SPONSORED RESEARCH

This invention was made with government support under Grant No. 2017-67007-25939 awarded by the United States Department of Agriculture, National Institute of Food and Agriculture. The government has certain rights in the invention.

### SEQUENCE LISTING

The instant application contains a Sequence Listing which has been submitted in ASCII format via EFS-Web and is hereby incorporated by reference in its entirety. Said ASCII copy, created on May 27, 2020 is named P1322WO00_SEQLISTING_06-17-2020_ST25 and is 385,059 bytes in size.

### BACKGROUND

Plant breeding and genetic engineering have been used now for many decades to improve plant performance and provide agricultural improvement of plants especially crop plants. A drawback in plant transformation techniques is the low regeneration efficiency of certain plants, including low efficiency in select plant genus and species and even particular genotypes within a plant species. Therefore, there is a need to improve regeneration efficiency of plants.

### SUMMARY

The present methods are to improving plant regeneration efficiency. The methods employ introducing into one or more plant cells a Growth Regulating Factor (GRF) and/or GRF-Interacting Factor protein (GIF) that are introduced into a plant cell, or a GRF-GIF chimera introduced into a plant cell. Introducing a nucleic acid molecule encoding the polypeptides or introducing the polypeptides increases regeneration efficiency. In certain embodiments, the methods increase regeneration efficiency of low regeneration efficiency plants. Other embodiments provide for accelerating the time to produce transgenic plants. Embodiments provide for mutating the miR396 target region of the GRF sequence, to reduce repression of the GRF protein by miR396 in plant cells. Still further embodiments provide that the presence of the GRF, and GIF or GRF-GIF chimera polypeptide in the plant cell allows the cell to be regenerated on media that has cytokinin concentration too low for regeneration of plant cells that do not comprise the polypeptides so introduced. Plants may be selected on such media without the need of an additional marker sequence. In certain embodiments, plants transformed with GRF and/or GIF or GRF-GIF that have high regeneration efficiency are used for subsequent transformation experiments. Operably linking the GRF, GIF or GRF-GIF chimera to an inducible promoter or domain also allows for selection of timing of expression or activity of the polypeptides. In certain embodiments, plants transformed with GRF and/or GIF or GRF-GIF that have high regeneration efficiency are used for subsequent transformation experiments. Still further embodiments provide that the GRF, GIF or GRF-GIF chimera are combined with gene editing technologies, and that the GRF, GIF or GRF-GIF chimera together with the gene editing constructs may be removed by segregation in the progeny of the edited plant.

### TECHNICAL FIELD

This disclosure relates generally to methods of plant regeneration. More specifically, this disclosure relates to methods for improving plant regeneration efficiency by transformation with a Growth-Regulating Factor (GRF) and/or a GRF-Interacting Factor (GIF), or a GRF-GIF chimera.

### BRIEF DESCRIPTION OF THE FIGURES

FIG. 1. A-C) Molecular Phylogenetic analysis using the Neighbor-Joining method. A) Arabidopsis, rice and wheat GRF proteins. B) Arabidopsis, rice and wheat GIF proteins. C) Scheme of the wheat GRF4-GIF1 chimera expressed by the maize UBIQUITIN promoter.

FIG. 2. Callus in cytokinins regeneration media. A higher number of shoots regenerated during Kronos transformation in the presence of the Ubi::GRF4-GIF1chimera (SEQ ID NO:5, middle) than in Kronos transformation using the same vector without the GRF-GIF chimera (shown at both sides).

FIG. 3. A) Validation of 26 independent transformation events by PCR. PCR products obtained with primers Fw-GRF4b and Rev-GIF1b using genomic DNA isolated from 26 independent T₀ plants as template. Each number correspond to an independent transformation event. L=DNA ladder. B) Schematic representation of the GRF4-GIF1 chimera and the primers used for genotyping. The amplified fragment is 1.087 Kb.

FIG. 4. Callus in cytokinins regeneration media. Shoot regeneration during wheat transformation in the presence of the Ubi::GIF1 (SEQ ID NO:2) and in wheat transformation using the same vector without the GIF1 gene.

FIG. 5. Callus in cytokinins regeneration media. Regeneration efficiency of plant cells transformed with Ubi::GRF4 (SEQ ID NO:1), Ubi::GIF1 (SEQ ID NO:2), Ubi::GRF4-GIF1chimera (SEQ ID NO:5) compared to control.

FIG. 6. Effect of GRF4-GIF1 chimera on regeneration from leaf explants.

FIG. 7. A) Schematic representation of the different steps of wheat transformation. B) Effect of GRF4-GIF1 chimera on induction of embryogenesis in the absence of cytokinins.

FIG. 8. Scheme of the wheat GRF4-GIF1 chimera expressed by the maize UBIQUITIN promoter (SEQ ID NO: 59). Below is the sequence of the wild-type miR396 target site (top (SEQ ID NO: 53), a miR396-resistant site with silent mutations (bottom (SEQ ID NO: 54), and their interaction with miR396 (middle (SEQ ID NO: 55).

FIG. 9. A) Shoot regeneration of plant cells transformed with a construct comprising GRF4-GIF1 chimera, Cas9, and a guide RNA (gRNA) targeting Gene Q. B) Region of the gene Q targeted with the guide RNA (SEQ ID NO: 68). C) Confirmation of the editing of the target gene.

FIG. 10. Shoot regeneration of plant cells transformed with GRF4-GIF1 chimera grown in one-tenth of the normal cytokinin concentration.

FIG. 11. Coding sequences of wheat chimeric clones. A) The nucleotide sequence of wheat GRF4-GIF1. Sequences are from tetraploid wheat Kronos. B) The sequence of the wheat GRF4-GIF1-encoded protein. Sequences in blue are from GRF4, in black for the spacer, and in green for GIF1.

FIG. 12. The sequence of pLC41- Ubi::GRF4-GIF1 (SEQ ID NO: 5). The GRF4 sequence is in blue letters, the spacer in black, and the GIF sequence is in green letters. The Ubi promoter is highlighted in gray, the HA tag in red, the NOS terminator in pink, the 35S promoter in green, and HPT in yellow. LB (GTTTACACCACAATATATCCTGCCA) (SEQ ID NO: 57) and RB (GTTTACCCGCCAATATATCCTGTCA) (SEQ ID NO: 58) sequences are underlined.

FIG. 13. The sequences of the Vitis vinifera GRF4-GIF1 chimera. A) The nucleotide sequence of Vitis GRF4-GIF1 (SEQ ID NO:6). B) The sequence of the Vitis GRF4-GIF1-encoded protein (SEQ ID NO:7). Sequences in blue are from GRF4, in black for the spacer, and in green for GIF1.

FIG. 14. A) Example sequence of a concatenated QLQ-WRC domain used for a BLASTP search and the phylogenetic analysis. B) The sequences of predicted proteins of the five selected wheat GRFs (SEQ ID NO: 9-13). Conserved QLQ and WRC domains are highlighted in yellow and green respectively.

FIG. 15. The sequences of predicted proteins of the five rice GRF orthologs (SEQ ID NO: 14-18). Conserved QLQ and WRC domains are highlighted in yellow and green respectively.

FIG. 16 shows A) the predicted sequences of the closest Arabidopsis GRFs (SEQ ID NO: 19-22) and B) the predicted protein of the closet Vitis vinifera GRF (SEQ ID NO: 23) Conserved QLQ and WRC domains are highlighted in yellow and green respectively.

FIG. 17 shows A) the predicted sequences of the closest Arabidopsis GIFs (SEQ ID NO:24, SEQ ID NO:25, and SEQ ID NO:26) and B) shows the predicted wheat GIF sequences (SEQ ID NO:27, SEQ ID NO:28, and SEQ ID NO:29). Conserved SNH domains are highlighted in yellow.

FIG. 18 are graphs showing wheat transformation efficiency using single wheat GRF4 or GIF1. A) Schemes of the different cassette used to express wheat GIF1 and GRF4 alone, GRF4 and GIF1 alone in the same T-DNA, and the GRF4-GIF1 chimera. B to D) Regeneration frequency of transgenic Kronos plants transformed with the wheat GRF4-GIF1 chimera relative to Kronos plants transformed with: B) empty vector (n=14, ****P<0.0001). C) Control vector and vectors including only GIF1 or only GRF4 (n=5, different letters indicate significant differences at P<0.05, Tukey Test). D) Vector including GRF4 and GIF1 under separate Ubi promoters (not fused, n=5, ** P<0.0144).

FIG. 19 shows phylogenetic trees of GRF and GIF families for wheat (yellow highlight), rice, Arabidopsis, citrus and grape. The closest homologs to wheat GRF4 and GIF1 are highlighted in orange for citrus and in violet for grape. We will refer to those genes and their encoded proteins as citrus GRF4 (Ciclev10032065m), citrus GIF1 (Ciclev10022144m), and grape GRF4 (GSVIVT01024326001) and grape GIF1 (GSVIVT01036262001). A) We used the QLQ and WRC domains for the analysis of the GRF proteins and B) the SNH domain for the analysis of the GIF proteins. The evolutionary history was inferred by using the Maximum Likelihood method. We show the tree with the highest log-likelihood. The percentage of trees in which the associated taxa clustered together is shown next to the branches. We conducted the evolutionary analysis in the program MEGA X. Yellow highlight: wheat. Orange highlight: selected Citrus homolog. Violet highlight: selected Vitis homolog.

FIG. 20. Shows wheat transformation with chimeras including other GRF and/or GIF sequences. A) Scheme of a GRFs-GIF1 chimeras tested. B) Regeneration efficiency induced by chimeras combining different wheat GRF genes with GIF1. Means are based on three experiments, except for GRF5 (2 experiments available). Different letters over the bars indicate significant differences with the control in a Tukey test (P<0.05). Only GRF4-GIF1 and GRF5-GIF1 were significantly different from the control. The asterisk indicates a significant difference in regeneration efficiency (P=0.0368) in a contrast comparing the combined GRF4-GIF1 and GRF5-GIF1 chimeras (evolutionary related) with the combined GRF1-GIF1 and GRF9-GIF1 chimeras (more distantly related, FIG. 19A). C) Scheme of a GRF4-GIFs chimeras tested. D) Regeneration frequency of transgenic Kronos plants transformed GRF4 chimeras fused to either GIF1, GIF2 or GIF3 (3 experiments, contrast chimeras with GIF1 vs. GIF2 and GIF3 P=0.0046). Different letters above bars indicate significant differences (P<0.05, Tukey test). Error bars are s.e.m. The number of inoculated embryos is indicated below the constructs. Normality of residuals was confirmed by Shapiro-Wilk's test and homogeneity of variances by Levene's test (raw-data available in Table 7).

FIG. 21. Shows effect of the GRF4-GIF1 chimera in regeneration efficiency in different genotypes. A) Representative transformations showing higher frequency of regenerated shoots in the presence of the GRF4-GIF1 chimera than in the control (empty vector) in different wheat and Triticale genotypes. B) Regeneration efficiency of GRF4-GIF1 vs control in different cultivars of tetraploid and hexaploid wheat and Triticale breeding line UC3190. The number of inoculated embryos is indicated below the names and the frequencies are indicated on top of the bars. The raw data is in Table 9.

FIG. 22. Shows A) Scheme of the inducible wheat GRF4-GR-GIF1 chimera with a rat Glucocorticoid Receptor (GR) in the middle of GRF4-GIF1 (SEQ ID NO:32, 33). C) Picture of plates containing transformed Kronos embryos in regeneration media in absence of DEX (−dex) or in the presence of 10 uM DEX (+dex). The presence of DEX induces GRF4-GR-GIF1 activity and significantly increases regeneration efficiency.

FIG. 23 shows the GRF4-GIF1 chimera induces embryogenesis in the absence of cytokinins. A) Schematic representation of the different steps of wheat transformation. B). Representative callus in auxin media with no hygromycin. Note growing green shoots in callus transformed with the wheat GRF4-GIF1 chimera in the absence of cytokinins (red arrows). Control: pLC41. C) Transgenic specific PCR product (arrow) shows no transgenic plants among four plants regenerated from the control and five transgenic plants among nine regenerated from the GRF4-GIF1 chimera. A pair of primers specific for the T-DNA was used in the PCR.

FIG. 24 shows the effect of the GRF4-GIF1 chimera in regeneration efficiency in the absence of exogenous cytokinin. Immature wheat embryos from a GRF4-GIF1 transgenic Kronos T₁ plant and a segregating non-transgenic T₁ sister line where treated following the standard transformation protocol, excluding the Agrobacterium inoculation and the addition of hygromycin to the plates. In the last step, the calli where transferred to regeneration media in the absence of cytokinin. The number of calli regenerating green shoots was significantly higher in the GRF4-GIF transgenic plant (21 out of 27) than in the non-transgenic sister control (3 out of 26). Pictures of representative plates showing calli in regeneration media without cytokinin.

FIG. 25 shows accelerated wheat transformation protocol using the GRF4-GIF1 chimera relative to normal protocol of wheat transformation at the UC Davis transformation facility. The protocol with the GRF4-GIF1 chimera is faster, reducing the overall process by 5 weeks.

FIG. 26 shows transformation of citrus with GRF4-GIF1 chimeras. A) Citrus epicotyls transformed with an empty vector and the Citrus GRF4-GIF1 chimera (60 d after Agrobacterium inoculation). B) Citrus epicotyls transformed with an empty vector and the Vitis GRF4-GIF1 and miR396-resistant GRF4-GIF1 (γGRF4-GIF1) chimeras (120 d after Agrobacterium inoculation). C) Scheme of a Vitis GRF4-GIF1 chimera showing the miR396 target site and its interaction with miR396 below. In the miR396-resistant a GRF4-GIF1 version, we introduced silent mutations (in red) to reduce interactions with miR396. D) Statistical comparison of the three Citrus experiments. Different letters indicate significant differences in a Tukey test (P<0.05). Horizontal lines on top indicate a significant contrast between the control and the GRF-GIF constructs (P=0.0153).

FIG. 27 shows transformation of grape with rGRF4-GIF1 chimera. The photo is of grape regenerating calli transformed with an empty vector and the grape rGRF4-GIF1 chimera.

FIG. 28 shows transformation of pepper (Capsicum annuum) cultivar R&C Cayenne with a GRF4.1-GIF1.1 chimera including Capsicum annuum closest homologs to wheat GRF4=pepper LOC107869915 (SEQ ID NO: 138) and to wheat GIF1=pepper LOC107870303 (SEQ ID NO: 139) respectively. The picture corresponds to Experiment #201027/28, showing a >4-fold increase in regeneration efficiency in the pepper cotyledon pieces transformed with the GRF4.1-GIF1.1 chimera (10 in 42=23.8%) compared with those transformed with the empty control vector (2 in 40=5.0%) (Table 11, Method 6).

### DETAILED DESCRIPTION

The present disclosure provides methods of using Growth-Regulating Factor proteins (GRFs) and GRF-Interacting Factor proteins (GIFs), either alone or in combination or as a fused chimera, to improve plant regeneration efficiency. The GRF genes belong to a conserved plant-specific family of transcription factors (TFs) (van der Knaap et al., 2000; Kim et al 2003). The GRF family is defined by the presence of the domains QLQ and WRC, which mediate protein-protein and protein-DNA interactions respectively (Kim et al., 2003, Kim and Kende 2004, Horiguchi et al., 2005). The GRF TFs are highly conserved in land plants, and GRF genes have been identified in dicots, monocots, gymnosperms and moss (Omidbakhshfard et al., 2015). These genes are also conserved targets of the microRNA miR396 (Debernardi et al., 2012). GRFs can control the size of many plant organs, and act as promoters of growth by increasing cell proliferation in developing organs (Rodriguez et al., 2010). Loss-of-function mutations in GRFs or downregulation by overexpression of miR396, can significantly reduce plant size in species like Arabidopsis and rice (Horiguchi et al., 2005; Kim et al., 2003; Kim and Kende, 2004; Wang et al., 2011; Liu et al., 2009; Rodriguez et al., 2010; Li S et al., 2016). On the other hand, increased GRF activity can produce larger organs, including larger leaves, grain and roots in Arabidopsis, rice, wheat, and brassicas, among others (Horiguchi et al., 2005; Rodriguez et al., 2010; Debernardi et al., 2014; Beltramino et al., 2018; Li, S. et al. 2018).

The GRF proteins can form complexes and work together with proteins encoded by members of the GIF gene family. GIF proteins do not have a DNA binding domain (Kim and Kende, 2004), but it has been demonstrated that GIF proteins interact with GRFs and with chromatin remodeling complexes in vivo (Debernardi et al., 2014; Vercruyssen et al., 2014). Based on that observation, it was proposed that GIFs could act as co-activators bringing chromatin remodeling complexes to DNA sequences recognized by the GRFs (Debernardi et al., 2014, Vercruyssen et al., 2014). Mutation in GIF genes mimic most of the phenotypes observed in GRF loss-of-function, while overexpression of GIF can promote organ growth and can boost the activity of GRFs (Kim and Kende 2004, Horiguchi et al., 2005; He et al., 2017; Shimano et al., 2018; Zhang et al., 2018; Debernardi et al., 2014). It has been previously observed that when Arabidopsis GRF3 and GIF1 are expressed together as a chimera, they can promote a larger increase of leaf size relative to the individual separate genes (patent WO 2013/102762 A1).

In an embodiment, the plant into which the GRF and/or GIF sequences are introduced is transgenic, that is, one which has had a heterologous nucleic acid molecule or polypeptide introduced, or which has had its genome edited by any of the techniques available including the examples provided herein. Such a heterologous nucleic acid molecule or polypeptide is any which is not naturally found next to the adjacent nucleic acid molecule or where the levels of polypeptide are higher or lower than that of a plant not comprising the heterologous nucleic acid molecule or polypeptide. In another example the nucleic acid molecule or polypeptide may be from another organism. When referring to a heterologous nucleic acid molecule it includes such a molecule linked to the promoter that does not naturally occur with the promoter sequence and/or is modified in genomic locus by human intervention. The nucleotide sequence in an example is heterologous to the promoter sequence, but it may be from any source, and it may be homologous or native and found naturally occurring in the plant cell, or heterologous or foreign to the plant host. Using the methods described here, a plant transformed with a GRF-GIF chimera or the GRF and/or GIF transgenes and the heterologous nucleic acid molecule or polypeptide has increased regeneration efficiency. The present disclosure provides methods that may significantly expand the number of plant species amenable to efficient transformation technology by significantly increasing plant regeneration efficiency. The methods provided herein may also be used to expand the genotypes that can be transformed within a certain crop. In wheat, for example, only a few cultivars including Bobwhite, Fielder and Kronos can be transformed efficiently, and those still show low regeneration efficiency. The ability to transform different genotypes is important for breeding applications, because the ability to transform directly top producing genotypes would eliminate the need of costly and lengthy backcrossing processes. The methods provided herein may be used to accelerate the production of transgenic plants from leaf explants instead of embryos.

In certain embodiments, the present disclosure provides methods of producing a plant having an improved regeneration efficiency. This method comprises the steps of (1) transforming one or more cells of the plant with one or more nucleic acid molecules, wherein the one or more nucleic acid molecules encode a GRF protein, a GIF protein, or a GRF-GIF chimera; and (2) culturing the one or more plant cells in regeneration media. Embodiments provide the GRF and GIF nucleic acid molecule or polypeptide may be introduced separately or may be introduced into the plant as a chimera. Constructs may comprise nucleic acid molecules encoding the chimera or multiple constructs may be introduced in embodiments.

The one or more cells of the plant may be transiently or stably transformed. The cells may be derived from any plant tissue, for example, from a leaf explant, microspore, ovule, etc. The cells may be protoplasts.

Embodiments provide that the time to produce a transgenic plant from the time of transformation is greatly reduced. The time to produce such a plant may be accelerated such that the time to produce the transgenic plant is decreased by five days, ten days, 15 days, 20 days, 25 days, 30 days, or more, or amounts in-between, compared to time to produce a transgenic plant where the GRF/GIF proteins are not introduced into the plant. For example, use of the proteins and nucleic acid molecules encoding same in wheat accelerates the production of transgenic plants from 90 to 60 days.

The term plant or plant material or plant part is used broadly herein to include any plant at any stage of development, or part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. The tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, provided are plants regenerated from the tissue cultures.

Improved or increased regeneration efficiency refers to increasing the number of plant cells, tissue or plants regenerated from the one or more cells having introduced the GRF, GIF or GRF/GIF chimera. In an embodiment the number of shoot regeneration and embryogenesis is increased. Further embodiments provide the regeneration can occur on media that has cytokinin concentration that is too low for the plant cell to regenerate. Using this type of method, it is possible to select for plants comprising the GRF, GIF or GRF-GIF chimera without the need for a separate marker to identify transformed cells. Plants that can grow on such media will comprise the molecules or proteins. In certain embodiments, the GRF protein can be a wheat GRF1, GRF2, GRF3, GRF4, GRF5, GRF6 or GRF9 polypeptide or the related proteins in other plant species. Embodiments provide in one example the GRF protein is wheat, rice, Arabidopsis, Vitis, Citrus or Capsicum GRF polypeptide. Still further embodiments provide the GRF protein is a wheat GRF4 or a protein that shares significant identity with a wheat GRF4, and the GIF is wheat GIF1 or a protein that shares significant identity with a wheat GIF1. Exemplary embodiments provide the GRF polypeptide may be as shown in any one of SEQ ID NO: 9-23, and 37-39, Examples of GIF sequences are any of those shown at SEQ ID NO: 24-30, 43-44, and 52. In some embodiments, the GRF is at least about 50%, at least about 60%, at least about 70%, at least about 80%, at least about 85%, at least about 90%, at least about 95%, or at least about 99% identical to the above listed sequences. In some embodiments, the GIF is at least about 50%, at least about 60%, at least about 70%, at least about 80%, at least about 90%, at least about 95%, or at least about 99% identical to the above listed sequences. In some embodiments, the one or more nucleic acid molecules encode a chimeric GRF-GIF construct in which the GRF portion is at least about 50%, at least about 60%, at least about 70%, at least about 80%, at least about 85%, at least about 90%, at least about 95%, or at least about 99% identical to a corresponding portion of a wheat GRF4 or other above listed GRF sequences and the GIF portion is at least about 50%, at least about 60%, at least about 70%, at least about 80%, at least about 85%, at least about 90%, at least about 95%, or at least about 99% identical to the corresponding portion of the above GIF sequences and in an embodiment to the wheat GIF1 sequence. In some embodiments, the one or more nucleic acid molecules encode a chimeric GRF4-GIF1 construct.

In some embodiments, the GRF protein comprises QLQ and WRC domains that are at least 70%, at least 80%, at least 85%, at least 90%, at least 95%, at least 99% identical to at least one of the domains as may be found in SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22. In other embodiments, the GRF comprises QLQ and WRC domains that are at least 70%, at least 85%, at least 90%, at least 95%, at least 99% identical to at least one of the QLQ domain of at least one of SEQ ID NO: 69-83 and the WRC domain of at least one of SEQ ID NO: 84-95 which are found in the full length GRF sequences of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22. In examples, QLQ domains are those as shown in SEQ ID NO: 69-83 and WRC domains are those as shown in SEQ ID NO: 84-95 and having such identity thereto.

In some embodiments, the GIF comprises an SNH domain that is at least 70%, at least 80%, at least 85%, at least 90%, at least 95%, at least 99% identical to at least one of SEQ ID NO: 97-103 which are found in the full length GIF sequences of SEQ ID NO:24, SEQ ID NO:25, SEQ ID NO:26, SEQ ID NO:27, SEQ ID NO:28, or SEQ ID NO:29 or SEQ ID NO: 133 (Os11g40100); SEQ ID NO: 134 (Os12g31350) or SEQ ID NO: 135 (Os03g52320). Examples of such domains are those found at SEQ ID NO: 97-103, 136 and 137nd having such sequence identity thereto.

In some embodiments, the one or more nucleic acid molecules encoding the GRF comprise one or more mutations in the recognition site of miR396. Further embodiments provided the mutations are silent mutations. Still further embodiments provide the mutations reduce repression of the GRF protein. Yet further embodiments provide the mutations are made to a miR396 target site as shown in wild type sequence of SEQ ID NO: 53 and in one example can produce the modified miR396 of SEQ ID NO: 54.

The term nucleic acid molecule refers to a nucleic acid molecule, which can be a RNA molecule as well as DNA molecule, and can be a molecule that encodes for a desired polypeptide or protein, but also may refer to nucleic acid molecules that do not constitute an entire gene, and which do not necessarily encode a polypeptide or protein. For example, when used in a homologous recombination process, the promoter may be placed in a construct with a sequence that is similar to an area of the chromosome in the plant that may not encode a protein. If desired, the nucleotide sequence of interest can be optimized for plant translation by optimizing the codons used for plants and the sequence around the translational start site for plants. Sequences resulting in potential mRNA instability can also be avoided.

Unless otherwise indicated, a particular nucleic acid sequence also implicitly encompasses conservatively modified variants thereof (e.g. degenerate codon substitutions) and complementary sequences as well as the sequence explicitly indicated. The term conservatively modified variants applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide is implicit in each described polypeptide sequence and is within the scope of the products and processes described.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994).

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., see, e.g., Creighton, Proteins: Structures and Molecular Properties (WH Freeman & Co.; 2nd edition (December 1993)).

In an embodiment the GRF polypeptide or nucleic acid molecule encoding same is selected from a wheat GRF1, GRF2, GRF3, GRF4, GRF5, GRF6, GRF9 polypeptide or nucleic acid molecule encoding same or homolog thereof. An embodiment provides the GIF polypeptide or nucleic acid molecule encoding same is selected from a wheat GIF1, GIF2 or GIF3 polypeptide or nucleic acid molecule encoding same or homolog thereof. Embodiments provide the polypeptide or nucleic acid molecule encoding same is a wheat GRF1, GRF2, GRF3, GRF4, GRF5, GRF6, GRF9 polypeptide or nucleic acid molecule encoding same or a homolog of such wheat polypeptide or nucleic acid molecule encoding same. Further embodiments provide the polypeptide or nucleic acid molecule encoding same is a wheat GIF1, GIF2 or GIF3 polypeptide or nucleic acid molecule encoding same or homolog thereof. A homolog is a gene or polypeptide inherited in different species retaining the same biological function. The GRF family is defined herein and are transcription factors having the conserved QLQ and WRC domains which mediate protein-protein and protein-DNA interactions, are highly conserved in land plants and identified in dicots, monocots, gymnosperms and moss, as discussed herein. GIF proteins interact with GRFs and have a conserved SNH domain. Such GRF and GIF polypeptides have the property of increasing regeneration efficiency, particularly when combined in a chimeric protein. Examples of such homologs are provided herein, such as those found in rice, Citrus, Vitis, Capsicum and Arabidopsis. A person of skill in the art can readily identify such homologs. For example, NCBI provides searching for homologs for a gene or the protein in other organisms. See ncbi.nlm.nih.gov/homologene. Further, such sequences include those that hybridize to the sequences of the GRF and GIF nucleic acid molecules or polypeptides shown herein under stringent hybridization conditions.

Methods of alignment of sequences for comparison are well known in the art. Thus, the determination of percent identity between any two sequences can be accomplished using a mathematical algorithm.

Optimal alignment of sequences for comparison can use any means to analyze sequence identity (homology) known in the art, e.g., by the progressive alignment method of termed “PILEUP” (Morrison, (1997) Mol. Biol. Evol. 14:428-441, as an example of the use of PILEUP); by the local homology algorithm of Smith & Waterman (Adv. Appl. Math. 2: 482 (1981)); by the homology alignment algorithm of Needleman & Wunsch (J. Mol. Biol. 48:443-453 (1970)); by the search for similarity method of Pearson (Proc. Natl. Acad. Sci. USA 85: 2444 (1988)); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins (1988), Gene 73: 237-244; Corpet (1988), Nucleic Acids Res. 16:10881-10890; Huang, Computer Applications in the Biosciences 8:155-165 (1992); and Pearson (1994), Methods in Mol. Biol. 24:307-331); Pfam (Sonnhammer (1998), Nucleic Acids Res. 26:322-325); TreeAlign (Hein (1994), Methods Mol. Biol. 25:349-364); MEG-ALIGN, and SAM sequence alignment computer programs; or, by manual visual inspection.

Another example of algorithm that is suitable for determining sequence similarity is the BLAST algorithm, which is described in Altschul et al, (1990) J. Mol. Biol. 215: 403-410. The BLAST programs (Basic Local Alignment Search Tool) of Altschul, S. F., et al., searches under default parameters for identity to sequences contained in the BLAST “GENEMBL” database. A sequence can be analyzed for identity to all publicly available DNA sequences contained in the GENEMBL database using the BLASTN algorithm under the default parameters.

Software for performing BLAST analyses is publicly available through the National Center for Biotechnology Information, www.ncbi.nlm.nih.gov/; see also Zhang (1997), Genome Res. 7:649-656 for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al (1990), J. Mol. Biol. 215: 403-410). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff (1992), Proc. Natl. Acad. Sci. USA 89:10915-10919) alignments (B) of 50, expectation (E) of 10, M=5, N=−4, and a comparison of both strands. The term BLAST refers to the BLAST algorithm which performs a statistical analysis of the similarity between two sequences; see, e.g., Karlin (1993), Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In an embodiment, GAP (Global Alignment Program) can be used. GAP uses the algorithm of Needleman and Wunsch (J. Mol. Biol. 48:443-453, 1970) to find the alignment of two complete sequences that maximizes the number of matches and minimizes the number of gaps. Default gap creation penalty values and gap extension penalty values in the commonly used Version 10 of the Wisconsin Package® (Accelrys, Inc., San Diego, Calif.) for protein sequences are 8 and 2, respectively. For nucleotide sequences the default gap creation penalty is 50 while the default gap extension penalty is 3. Percent Similarity is the percent of the symbols that are similar. Symbols that are across from gaps are ignored. A similarity is scored when the scoring matrix value for a pair of symbols is greater than or equal to 0.50, the similarity threshold. A general purpose scoring system is the BLOSUM62 matrix (Henikoff and Henikoff (1993), Proteins 17: 49-61), which is currently the default choice for BLAST programs. BLOSUM62 uses a combination of three matrices to cover all contingencies. Altschul, J. Mol. Biol. 36: 290-300 (1993), herein incorporated by reference in its entirety and is the scoring matrix used in Version 10 of the Wisconsin Package® (Accelrys, Inc., San Diego, Calif.) (see Henikoff & Henikoff (1989) Proc. Natl. Acad. Sci. USA 89:10915).

(c) As used herein, “sequence identity” or “identity” in the context of two nucleic acid sequences makes reference to the residues in the two sequences that are the same when aligned for maximum correspondence over a specified comparison window.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

When referring to hybridization techniques, all or part of a known nucleotide sequence can be used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as 32P, or any other detectable marker. Thus, for example, probes for hybridization can be made by labeling synthetic oligonucleotides based on the DNA sequences. Methods for preparation of probes for hybridization and for construction of cDNA and genomic libraries are generally known in the art and are disclosed (Sambrook et al., 2001).

For example, the sequence disclosed herein, or one or more portions thereof, may be used as a probe capable of specifically hybridizing to corresponding sequences. To achieve specific hybridization under a variety of conditions, such probes include sequences that are unique among the sequences to be screened and are preferably at least about 10 nucleotides in length, and most preferably at least about 20 nucleotides in length. Such sequences may alternatively be used to amplify corresponding sequences from a chosen plant by PCR. This technique may be used to isolate sequences from a desired plant or as a diagnostic assay to determine the presence of sequences in a plant. Hybridization techniques include hybridization screening of DNA libraries plated as either plaques or colonies (Sambrook et al., 2001).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 0.1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem., 138:267-284 (1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of the complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with ˜90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (2001) Molecular Cloning: A Laboratory Manual (3rd ed., Cold Spring Harbor Laboratory Press, Plainview, N.Y.) and Haymes et al. (1985) In: Nucleic Acid Hybridization, a Practical Approach, IRL Press, Washington, D.C.

The term introduced in the context of inserting a nucleic acid or polypeptide into a cell, includes transfection or transformation or transduction and includes in embodiments reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA). As discussed below, introduction of proteins into plants can utilize methods such as those described in Example 8.

Various methods of transformation/transfection are available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed. Utilizing the methods here, increased regeneration efficiency is provided.

By way of example without limitation, methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993) Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580. In certain embodiments, the regulatory sequence comprises an inducible promoter. In other embodiments the activity of GRF, GIF or the GRF-GIF chimera is regulated by an inducible system. The inducible system may be or may include a glucocorticoid receptor fused to the GRF, GIF or GRF-GIF proteins. Other inducible systems may also be used.

In certain embodiments described herein, the one of more transformed plant cells may be cultured in regeneration media that comprises a suboptimal concentration of exogenous cytokinins. The term “suboptimal concentration” is defined as any concentration that is too low to allow adequate regeneration of plant cells that are not transformed with a GRF-GIF chimera or the GRF and/or GIF transgenes. A suboptimal concentration may be, e.g., less than about 50%, less than about 10%, less than about 5%, less than about 1%, or less than 0.01% of the cytokinin concentration than is typically used for plant regeneration. Cytokinin concentrations can be tested to determine an optimal concentration that permits regeneration of plant cells transformed with the GRF-GIF chimera but that is not sufficient to induce regeneration of non-transgenic plants. This can be used as a positive selection method to identify transgenic shoots without using antibiotic markers.

In certain embodiments, the methods disclosed herein increase regeneration efficiency. Regeneration efficiency refers to the number of plant cells that can be regenerated. The methods provide that efficiency is increased compared to regeneration in which a GRF/GIF polypeptide or nucleotide is not introduced into the plant. The regeneration efficiency can be increased by 1%, 2%, 3%, 4%, 5%, 10%, 15%, 20%, 25%, 30%, 40%, 45%, 50%, 55%, 60%, 65%, 70%, 80%, 90% or more or amounts in-between. By way of example without limitation a plant having 10% regeneration efficiency without use of GRF/GIF can be increased to 50-70%. A plant having 1% efficiency can be increased to 10-20% and a plant that has zero regeneration efficiency can be increased to 1-5%.

In some embodiments, the one or more nucleic acid molecules used in the methods disclosed here may, in addition to encoding a GRF and a GIF, encode at least one additional polynucleotide of interest.

The methods disclosed herein may be used in a variety of plants. In certain embodiments, the methods may be used in a plant that has a low regeneration efficiency. In certain embodiments, the plant is a monocot species. In certain other embodiments, the plant is a dicot species. In yet other embodiments, the plant is neither a monocot nor a dicot species. Example species include but are not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), Triticale ((x Triticosecale, a cross of wheat and rye), Triticale (x Triticosecale, a cross of wheat and rye), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos mucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), pepper (Capsicum annuum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present invention include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

The plant in preferred embodiments may be selected from a species of Oryza, Gossypium, Glycine, Vitis, Medicago, Juglans, Citrus, Capsicum, Sorghum, Zea, Hordeum or Triticum or other appropriate plant species. In certain embodiments, the plant is Triticum turgidum or Triticum aestivum.

The present disclosure also provides plants that are produced by any of the methods described herein.

The present disclosure provides methods to modulate the expression of GRF and GIF genes in plants to improve regeneration efficiency and to allow regeneration in the absence of exogenous cytokinin. These methods may be practiced by increasing the protein levels and activities of GRF and/or GIF or GRF-GIF chimera in a plant, plant cell, or protoplast.

In some embodiments, the GRF genes and GIF genes may be increased in a plant or plant cell by various means, including using vectors that each have only a GRF gene or a GIF gene, or a vector with a GRF-GIF chimera. The GRF gene may be mutated to render it less sensitive to the repression by miR396. The one or more nucleic acid molecules that encode a Growth-Regulating Factor protein (GRF) and/or a GRF-Interacting Factor protein (GIF) may be operably linked to a regulatory sequence operable in the plant cell. The genes in the vectors may be controlled by various types of promoters, including inducible, tissue-specific, and constitutive promoters. The plant or plant cell may be transformed by various means, including by using Agrobacterium harboring a vector with GRF and GIF genes or bombardment.

Gene editing may be used in combination with the present methods. For example, a vector with GRF and/or GIF, or GRF-GIF chimera and Cas9 may be used for gene editing of a plant or plant cell. The plants or plant cells may be transformed to express the nucleic acids transiently or stably. GRF and/or GIF, or GRF-GIF chimera proteins may be delivered to plants or plant cells to increase regeneration efficiency using methods described by other investigators to deliver CRISPR/Cas9 preassembled ribonucleoprotein complexes (RNPs) (Woo et al., 2015; Subburaj et al., 2016; Malnoy et al., 2016; Kim et al., 2017; Liang et al., 2017; Svitashev et al., 2016; Wolter et al., 2017).

Methods which provides for targeting of a molecule of interest (MOI) to the target site of the target gene may be utilized in the method. The following is provided by way of example rather than limitation. A guide nucleic acid molecule is one that directs the nuclease to the specific cut site in the genome, whether via use of a binding domain, recognition domains, guide RNAs or other mechanisms. The guide nucleic acid molecule is introduced into the cell under conditions appropriate for operation of the guide nucleic acid molecule in directing cleavage to the target locus. A person of skill in the art will have available a number of methods that may be used, the most common utilizing a nuclease to cleave the target region of the gene, along with sequences which will recognize sequences at the target locus and direct cleavage to the locus. Any nuclease that can cleave the phosphodiester bond of a polynucleotide chain may be used in the methods described here. By way of example without limitation, available systems include those utilizing site specific nucleases (SSN) such as ZFNs (Zinc finger nuclease), Whyte, et al. Cell Biology Symposium: Zinc finger nucleases to create custom-designed modifications. J Anim Sci 90, 1111-1117 (2012)); TALENs (Transcription activator-like effector nucleases) (see, Carlson, D. F. et al. Efficient TALEN-mediated gene knockout in animals. Proc Natl Acad Sci USA 109, 17382-17387 (2012); Tan, W. et al. Efficient nonmeiotic allele introgression in livestock using custom endonucleases. Proc Natl Acad Sci USA 110, 16526-16531 (2013); and the CRISPR (Clustered regularly interspaced short palindromic repeats)-associated (Cas) nuclease system (Hai, T., Teng, F., Guo, R., Li, W. & Zhou, Q. One-step generation of knockout pigs by zygote injection of CRISPR/Cas system. Cell Res 24, 372-375 (2014)) that have permitted editing of animal genomes. The use of recombinases such as FLP/FRT as described in U.S. Pat. No. 6,720,475, or CRE/LOX as described in U.S. Pat. No. 5,658,772, can be utilized to integrate a polynucleotide sequence into a specific chromosomal site. Meganucleases have been used for targeting donor polynucleotides into a specific chromosomal location as described in Puchta et al., PNAS USA 93 (1996) pp. 5055-5060. ZFNs work with proteins or domains of proteins binding to a binding domain having a stabilized structure as a result of use a zinc ion. TALENs utilize domains with repeats of amino acids, which can specifically recognize a base pair in a DNA sequence. For a discussion of both systems, see Voytas et al. U.S. Pat. No. 8,697,853, incorporated herein by reference in its entirety. These systems utilize enzymes prepared for each target sequence.

In referring to a target gene or molecule is meant to refer to any nucleic acid molecule within the genome desired to be modified as described or where it is desired to delete or insert a nucleic acid molecule or modify the molecule in some manner. Where the target molecule is a nucleic acid sequence, the target molecule can, for example, be a sequence coding a gene product (e.g., a protein) or a non-coding sequence (e.g., a regulatory polynucleotide or a junk DNA).

The term “regeneration” as used herein means generation of a plant or plant cells from another plant or plant cell. This includes the generation of shoots from calli generated from a plant embryo or young leaflets. In some embodiments, plants, plant cells, or protoplasts may be regenerated from plants, plant cells, or protoplasts that have been transformed to have increased GRF and/or GIF or GRF-GIF chimera expression. In some embodiments, plants, plant cells, or protoplasts regenerated from plants, plant cells, or protoplasts that have been transformed to have increased GRF and/or GIF or GRF-GIF chimera expression may be further transformed with at least one additional polypeptide.

In various embodiments, the plant or plant cell may originate from various plant species, including those resistant to transformation, having low regeneration efficiency, and those in Triticum, Vitis, Oryza, Gossypium, Citrus, Capsicum, Sorghum, and Juglans genus.

The present disclosure also provides methods to regenerate a plant or plant cell in the absence of exogenous cytokinins. In certain embodiments, such methods comprise introducing a GRF gene and/or a GIF gene, or a GRF-GIF chimera into plant cells and culturing the cells in medium substantially free of exogenous cytokinins to regenerate a plant. The GRF and GIF genes may be of Triticum aestivum, Triticum turgidum ssp. durum or of another plant species, including those that have low regeneration efficiency or are resistant to transformation.

In various embodiments, a plant may be transformed using vectors that each have only a GRF gene or a GIF gene, a vector with a GRF and GIF as a chimera, or a vector with a GRF and a GIF in a non-chimeric form.

In certain embodiments, the plant is or is considered recalcitrant to regeneration.

The nucleic acid molecule or polypeptide may be introduced into the plant along with other components. As used herein, a nucleotide segment is referred to as operably linked when it is placed into a functional relationship with another DNA segment. For example, DNA for a signal sequence is operably linked to DNA encoding a polypeptide if it is expressed as a preprotein that participates in the secretion of the polypeptide; a promoter or enhancer is operably linked to a coding sequence if it stimulates the transcription of the sequence. Operably linked elements may be contiguous or non-contiguous. When used to refer to the joining of two protein coding regions, by operably linked it is intended that the coding regions are in the same reading frame. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette can include one or more enhancers in addition to a promoter. By enhancer is intended a cis-acting sequence that increases the utilization of a promoter. Such enhancers can be native to a gene or from a heterologous gene. Further, it is recognized that some promoters can contain one or more enhancers or enhancer-like elements. An example of one such enhancer is the 35S enhancer, which can be a single enhancer, or duplicated. See for example, McPherson et al, U.S. Pat. No. 5,322,938.

The terms promoter, promoter region or promoter sequence refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease Si), as well as protein binding domains (consensus sequences) responsible for the binding of RNA polymerase.

Spatial and temporal control is also often important in driving gene expression in plants. For example, selectable and scoreable markers must be expressed at a suitable time and in an appropriate tissue to allow for screening and controlling enzymes and regulatory factors must be produced in metabolically active and physiologically responsive tissues, respectively. Similarly, genes conferring host protection must be expressed in the target tissues for the pathogen or pest, and plant produced protein products should be expressed in tissues suitable for protein accumulation and storage. Furthermore, since certain protein products may have detrimental effects on plant health and yield when expressed in metabolically active plant tissues that are essential for survival and growth, promoters may be favored that are active in the chosen plant storage tissues but show low or no activity in other, non-storage tissues.

In the methods, a number of promoters that direct expression of a nucleic acid molecule in a plant can be employed. Such promoters can be selected from constitutive, chemically-regulated, inducible, tissue-specific, and seed-preferred promoters. Constitutive promoters include, for example, the core CaMV 35S promoter (Odell et al. (1985) Nature 313:810-812); rice actin (McElroy et al. (1990) Plant Cell 2:163-171); ubiquitin (European patent application no. 0 342 926; Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984)EMBO J. 3:2723-2730), the core promoter of the Rsyn7 promoter and other constitutive promoters disclosed in WO 99/43838 and U.S. Pat. No. 6,072,050; to name a few.

A skilled person appreciates a promoter sequence can be modified to provide for a range of expression levels of and operably linked heterologous nucleic acid molecule. Less than the entire promoter region can be utilized and the ability to drive expression retained. However, it is recognized that expression levels of mRNA can be decreased with deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

Tissue-preferred promoters can be utilized to target enhanced transcription and/or expression within a particular plant tissue. When referring to preferential expression, what is meant is expression at a higher level in the particular plant tissue than in other plant tissue. Examples of these type of promoters include seed preferred expression such as that provided by the phaseolin promoter (Bustos et al. (1989) The Plant Cell Vol. 1, 839-853), and the maize globulin-1 gene, Belanger, et al. (1991) Genetics 129:863-972.

The range of available plant compatible promoters includes inducible promoters. Any inducible promoter can be used in the instant process. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)) Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

A cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; and the light-inducible promoter derived from the pea rbcS gene or pea psaDb gene (Yamamoto et al. (1997) Plant J. 12(2):255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90(20):9586-9590; Orozco et al. (1993) Plant Mol. Bio. 23(6):1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible regulatory element also can be the promoter of the maize Int-1 or Int-2 gene, which responds to benzenesulfonamide herbicide safeners (Hershey et al., Mol. Gen. Gente. 227:229-237, 1991; Gatz et al., Mol. Gen. Genet. 243:32-38, 1994), and the Tet repressor of transposon Tn10 (Gatz et al., Mol. Gen. Genet. 227:229-237, 1991). Stress inducible promoters include salt/water stress-inducible promoters such as P5CS (Zang et al. (1997) Plant Sciences 129:81-89); cold-inducible promoters, such as, cor15a (Hajela et al. (1990) Plant Physiol. 93:1246-1252), cor15b (Wilhelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338), and the heat-shock inducible element from the parsley ubiquitin promoter (WO 03/102198). Other stress-inducible promoters include rip2 (U.S. Pat. No. 5,332,808 and rd29a (Yamaguchi-Shinozaki et al. (1993) Mol. Gen. Genet. 236:331-340). Certain promoters are inducible by wounding, including the Agrobacterium pmas promoter (Guevara-Garcia et al. (1993) Plant J. 4(3):495-505) and the Agrobacterium ORF 13 promoter (Hansen et al., (1997) Mol. Gen. Genet. 254(3):337-343).

In addition, markers that facilitate identification of a plant cell containing the polynucleotide encoding the marker may be employed. Scorable or screenable markers are useful, where presence of the sequence produces a measurable product and can produce the product without destruction of the plant cell. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. (1987) The EMBO Journal vol. 6 No. 13 pp. 3901-3907); alkaline phosphatase. Other screenable markers include the anthocyanin/flavonoid genes in general (See discussion at Taylor and Briggs, (1990) The Plant Cell 2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., (1996) Plant Cell 8: 1171-1179; Scheffler et al. (1994)Mol. Gen. Genet. 242:40-48) and maize C2 (Wienand et al., (1986) Mol. Gen. Genet. 203:202-207); the B gene (Chandler et al., (1989) Plant Cell 1:1175-1183), the p1 gene (Grotewold et al, (1991 Proc. Natl. Acad. Sci USA) 88:4587-4591; Grotewold et al., (1994) Cell 76:543-553; Sidorenko et al., (1999) Plant Mol. Biol. 39:11-19); the bronze locus genes (Ralston et al., (1988) Genetics 119:185-197; Nash et al., (1990) Plant Cell 2(11): 1039-1049), among others. As discussed herein, when using the present invention with a culture media that has lower cytokine levels that necessary for regeneration of the plant cell, use of the GRF or GIF or GRF-GIF chimera may be utilized without an additional selectable marker.

A wide variety of other components such as signal sequences and polyadenylation sequences may be included with the vector as is desired.

### EXAMPLES

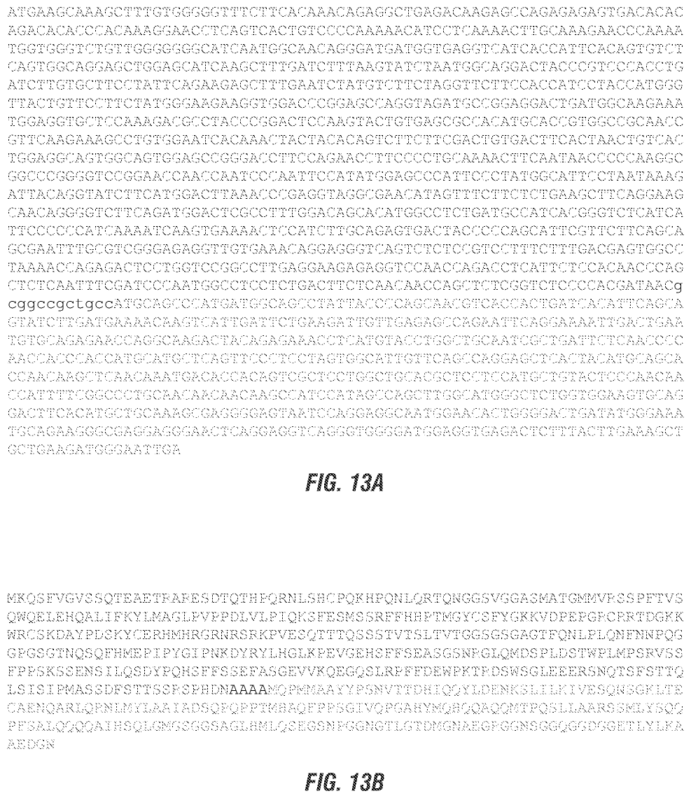

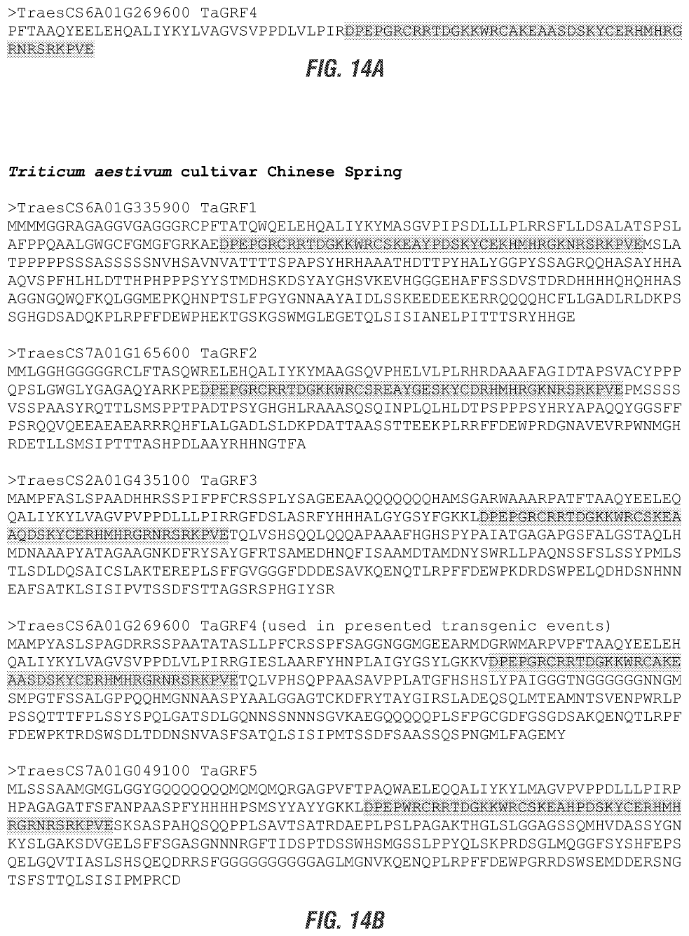

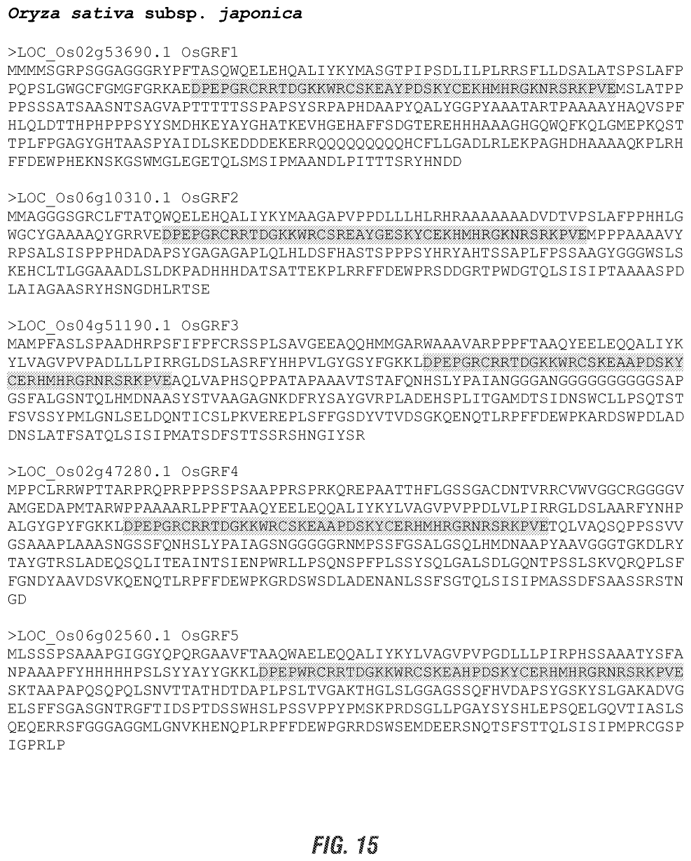

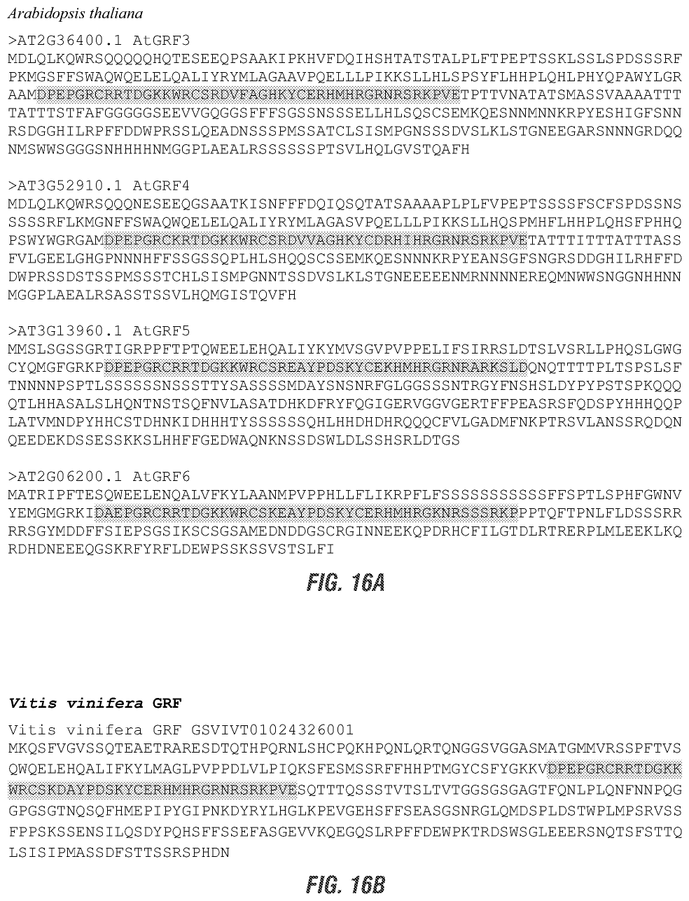

The following examples are for illustration only. In light of this disclosure, those of skill in the art will recognize that variations of these examples and other embodiments of the disclosed subject matter are enabled without undue experimentation. In reference to experiments herein, see FIG. 14 showing predicted proteins of selected wheat GRF genes GRF1-5 (SEQ ID NO: 9-13). Wheat gene names are based on Chinese Spring RefSeq v1.0. GRF numbers are based on rice orthologs. Only the sequences of the wheat A genome homeologs are provided (B and D genome are more than 90% identical). The encoded protein sequences of the closest wheat, rice and Arabidopsis homologs are indicated in SEQ ID NO: 9-22, with the conserved QLQ and WRC domains highlighted in yellow and green respectively. Predicted proteins of five rice GRF orthologs are shown in FIG. 15 (SEQ ID NO: 13-18). Predicted proteins of the closest Arabidopsis GRF3-6 are shown in FIG. 16A (SEQ ID NO: 19-22). Predicted protein of the closest Vitis vinifera GRF used in the chimera is shown in FIG. 13B (SEQ ID NO: 23).

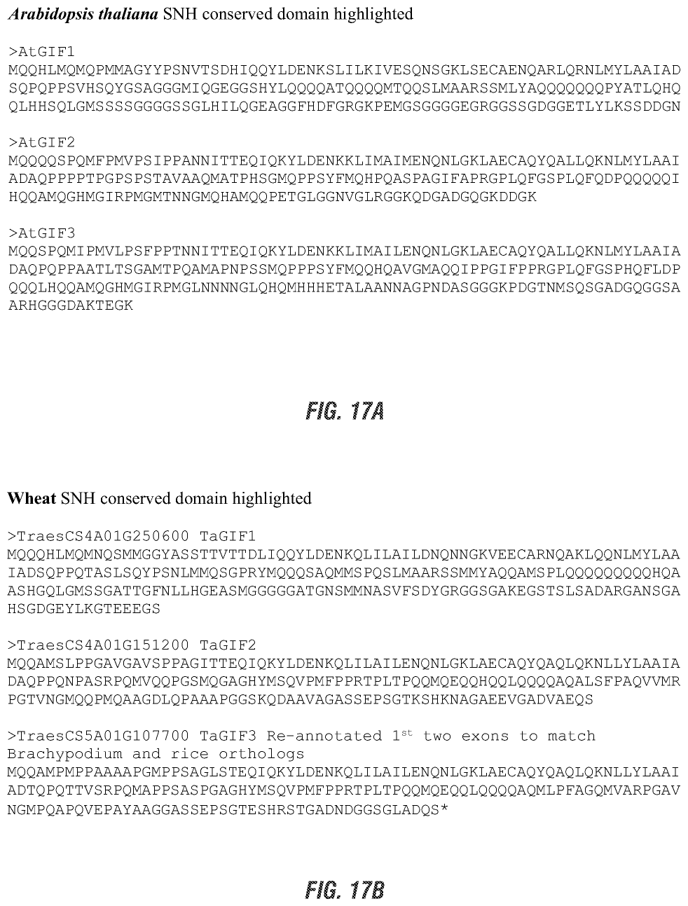

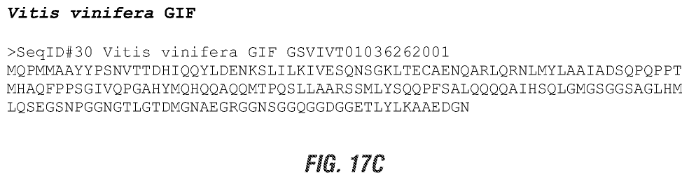

FIG. 17A shows predicted proteins of Arabidopsis GIF 1 and GIF2 (SEQ ID NO: 24-26) and 17B shows Triticum aestivum GIF1, GIF2 and GIF3 (SEQ ID NO: 27-29). Wheat gene names are based on Chinese Spring RefSeq v1.0. GIF numbers are based on rice orthologs. Only the sequences of the wheat A genome homeologs are provided (B and D genome are more than 90% identical). The encoded protein sequences of the closest Arabidopsis and wheat homologs are indicated below, with the conserved SNH domain highlighted in yellow. Vitis vinifera GIF is shown in FIG. 17C (SEQ ID NO: 30).

### Example 1—Transformation of Wheat with a GRF-GIF Chimera

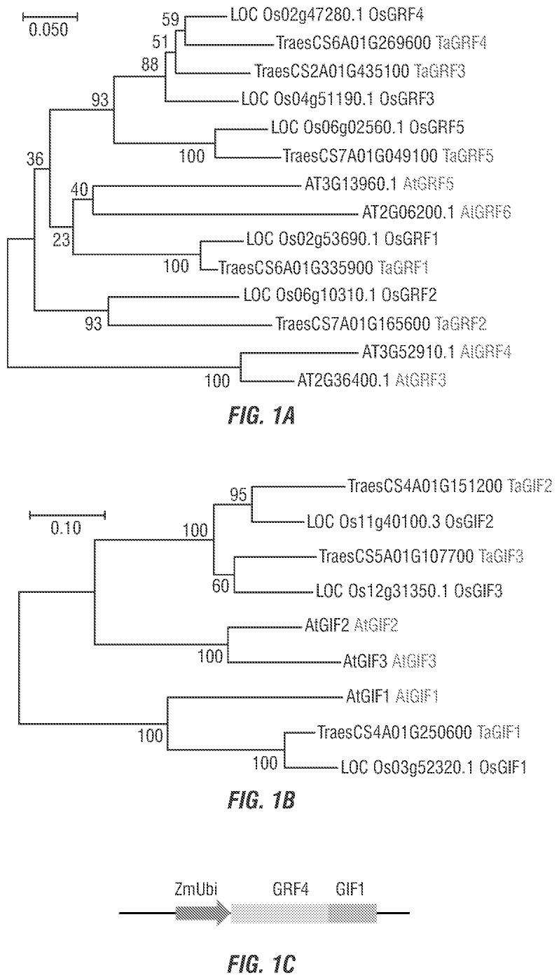

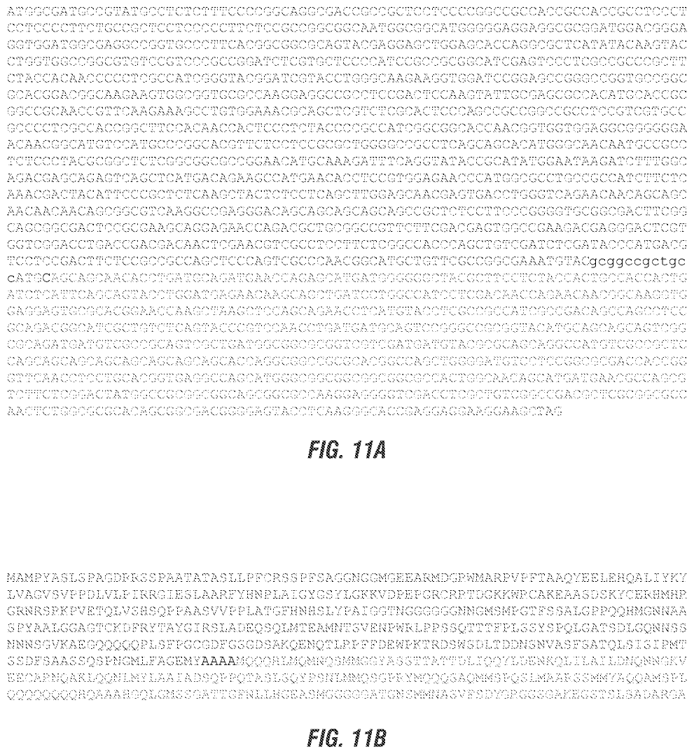

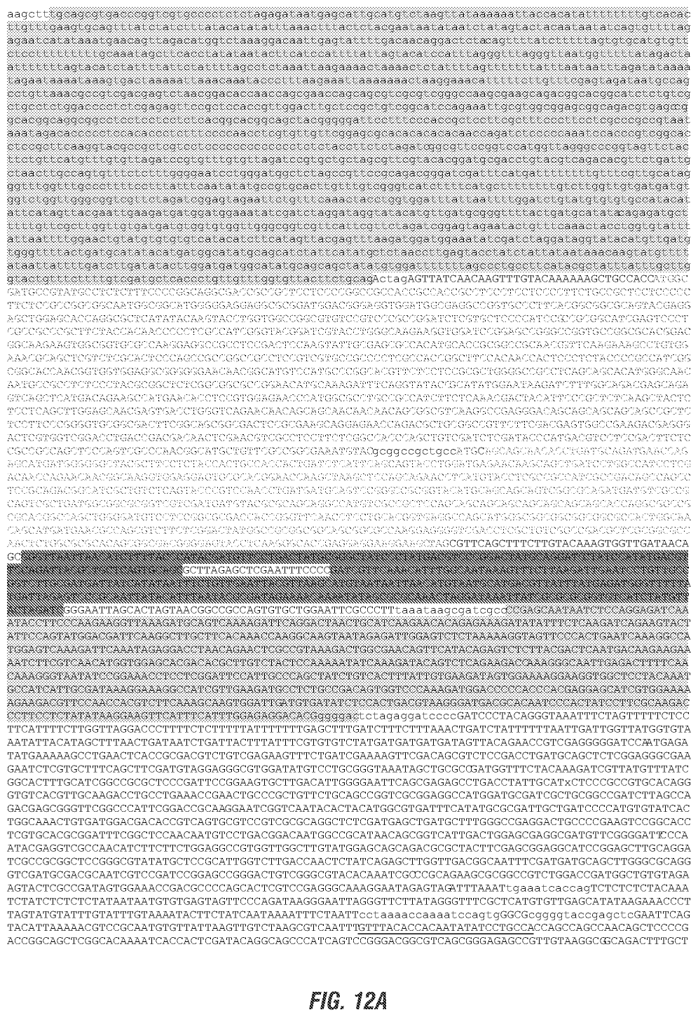

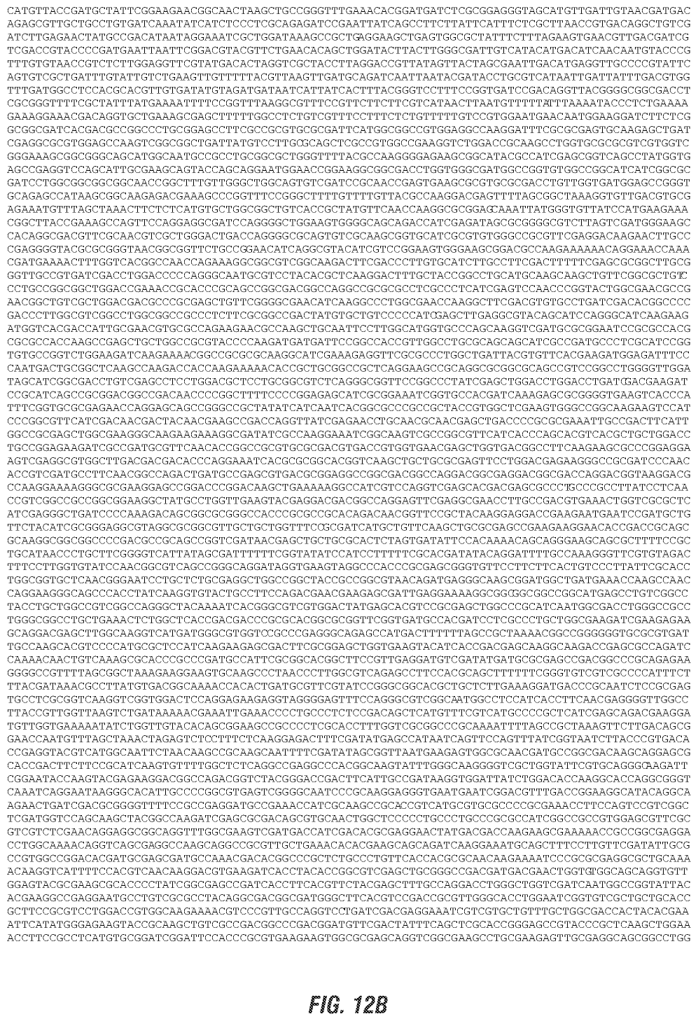

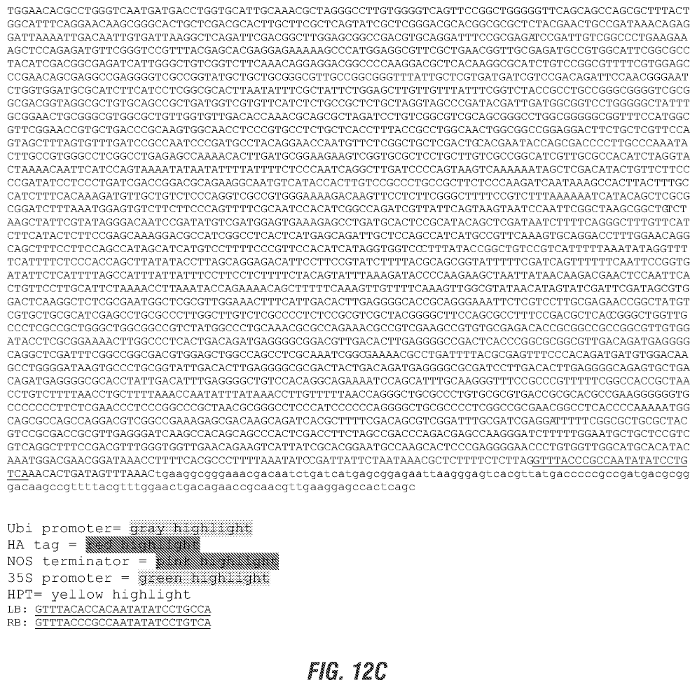

In an effort to increase plant biomass to promote increases in grain yield of agriculturally important crops, we tested the effects of GRF, GIF and GRF-GIF chimeras on wheat growth. Transgenic plants that expressed a wheat GRF-GIF chimera under the maize UBIQUITIN promoter were generated. We identified 10 GRFs and 3 GIFs in the wheat genome by Molecular Phylogenetic analysis using the Neighbor-Joining method (FIG. 1). To generate the wheat GRF-GIF chimera, we selected the wheat GRF homologue to the rice OsGRF4 (FIG. 1A), which promotes grain and plant growth in rice (Duan P et al., 2015; Hu J et al., 2015; Che R et al., 2015; Sun P et al 2016; Li, S. et al. 2018). For the GIF partner, we selected the closest homologue of Arabidopsis GIF1 (FIG. 1B), since members of this clade has been shown to control growth in Arabidopsis, rice and maize (Kim and Kende 2004, Horiguchi et al., 2005; Shimano S et al., 2018; Zhang D et al., 2018). The amino acid sequences of the SNH conserved domain were used to perform the phylogenetic analysis in FIG. 1B. The wheat closest homologue of Arabidopsis GIF1 is highlighted in bold. A schematic representation of the chimeric construct is in FIG. 1C. The amino acid sequence of the GRF and GIF genes from Arabidopsis, rice and Brachypodium were obtained from phytozome (https://phytozome.jgi.doe.gov/pz/portal.html). The amino acid sequences of wheat GRF and GIF genes were obtained from the wheat genome RefSeq v1.0. The phylogenetic analysis was performed using MEGA6. The coding sequence for the wheat chimeric GRF4-GIF1 is provided in FIG. 11A and the protein sequence in FIG. 11B. The sequence of the construct used for transformation, pLC41- Ubi::GRF4-GIF1, is provided in FIG. 12.

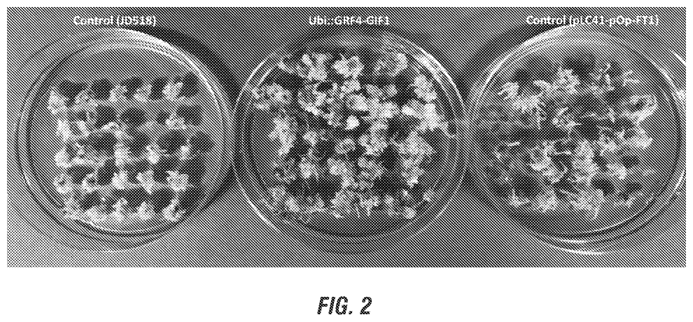

We then transformed the wheat GRF4-GIF1 chimera into tetraploid wheat Kronos (Triticum turgidum ssp. durum) by Agrobacterium-mediated transformation. The plant cells infected with Agrobacterium harboring the GRF4-GIF1 binary vector (a schematic representation of the construct shown in FIG. 1C) regenerated a surprising number of shoots, which was significantly higher than the number of shoots obtained in control parallel transformations with vectors having the same pLC41 backbone (FIG. 2). The GRF and GIF genes have not been previously associated with increased regeneration efficiency in transgenic plants.

We next analyzed the frequency of callus pieces regenerating shoots in two independent wheat transformation experiments using the GRF4-GIF1 chimera and two experiments without the chimera. The statistical analysis demonstrated that the average number of callus pieces regenerating shoots was more than 15-fold higher (P=0.0019) in the callus transformed with the GRF4-GIF1 chimera (92.8%±3.2%) than in the callus transformed with constructs without the GRF4-GIF1 chimera (6.0%±2%). The last numbers are representative of Kronos wheat transformations, where usually only one or two shoots are recovered from plates containing 25 callus (FIG. 2, both sides). For the wheat GRF4-GIF1 chimera (FIG. 2, middle), we recovered around 50 shoots per plate containing 25 callus, a substantial increase in the efficiency of regeneration of transgenic wheat plants.

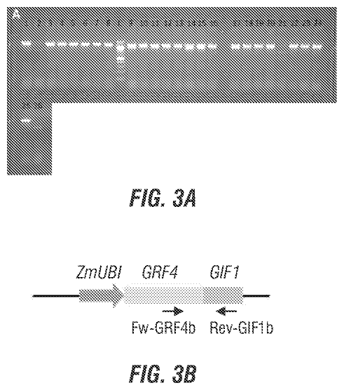

We transferred to soil 26 independent T₀ plants to verify the presence of the GRF4-GIF1 chimera. PCR amplification of genomic DNA from the 26 independent T₀ plants using primers Fw-GRF4b (5′-CCTCCGACTCCAAGTATTGC-3′) (SEQ ID NO: 104) and Rev-GIF1c (5′-ATCATCAGGTTGGACGGGTA-3′) (SEQ ID NO: 105) confirmed that 23 out of the 26 To plants had the GRF4-GIF1 transgene inserted in the genomic DNA (FIG. 3). The transgenic plants were fertile and showed a normal phenotype. Without being bound by any particular theory, this may be because the endogenous miR396 controlled the levels of the overexpressed GRF4-GIF1 chimera at later stages of plant development.

### Example 2—Transformation of Wheat Using Only GRF4 or GIF1

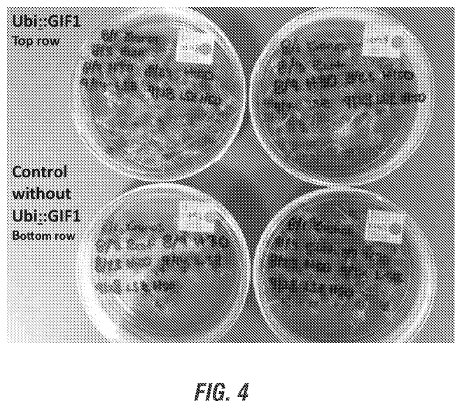

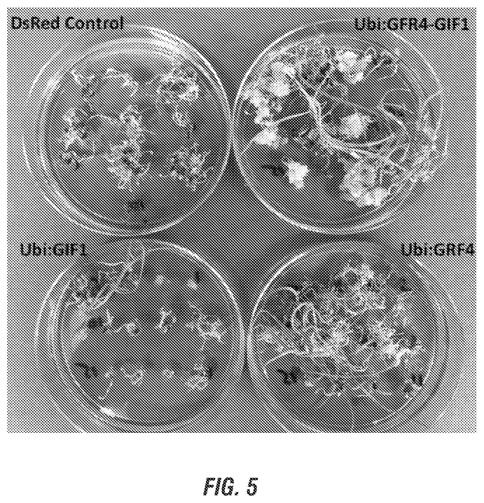

Wheat transformation experiments were performed using only GRF4 (SEQ ID NO:1) or only GIF1 (SEQ ID NO:2) in the same pLC41 vector as described for the GRF4-GIF1 chimera. We also observed a significant increase in regeneration efficiency relative to the control (FIGS. 4 and 5). The increase in regeneration efficiency was lower than that observed in the transformation experiments with the chimeric GRF4-GIF1. All regenerated plants were confirmed as transgenic by PCR. We also confirmed that all regenerated plants overexpressed the corresponding transgene by qRT-PCR of T₁ plants

### Example 3—Transformation of Other Plant Species or Tissues Using Only GRF4 or GIF1

In wheat, the GRF4-GIF1 chimera resulted in significantly higher regeneration efficiency than either of the two genes alone. It is, however, possible that in other plant species or tissues transformation with only one of these two genes might be sufficient to improve regeneration efficiency to a similar level as for plants transformed with the chimera. For example, if one of these genes is expressed at sufficiently high levels in a particular plant, transformation with the other gene alone could be sufficient to achieve significantly higher regeneration efficiency in that plant.

### Example 4—Regeneration from Leaf Explants

Clones of five independent GRF4-GIF1 To wheat lines and one DsRed T₀ control line were generated and maintained in culture. Very young leaf blades were aseptically dissected under a stereomicroscope and the outer 1-2 whorls of leaves removed. The base of the inner laminas were removed and plated onto a callus induction medium containing auxins to induce callus formation. After 14 days, tissue was sub-cultured to fresh callus induction medium. After an additional 21 days, calli were transferred to shoot regeneration media containing cytokinin. The number of calli regenerating shoots was scored after 28 days.

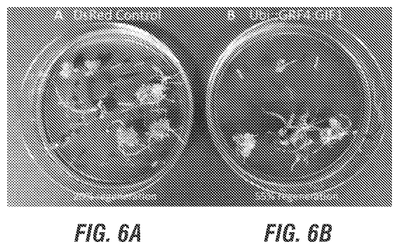

Leaf explants transformed with the GRF4-GIF1 chimera showed a regeneration efficiency of 55%, which was 2.8-fold higher than the regeneration efficiency in leaf explants transformed with constructs without the GRF4-GIF1 chimera (FIG. 6).

### Example 5—Regeneration of Plants in Suboptimal Levels of Exogenous Cytokinins

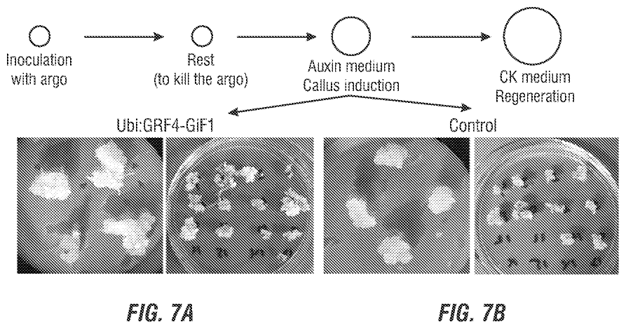

In regular transformations, immature embryos were first inoculated with Agrobacterium, and then placed through different media containing different hormones to regenerate the transgenic plants. Initially, a medium containing auxin induces callus formation. Next, the calli are transferred to media containing cytokinin, which promotes shoot development (FIG. 7A). Interestingly, we observed that embryos inoculated with Agrobacterium with the wheat GRF4-GIF1 chimera (SEQ ID NO:5) were able to regenerate green shoots in the auxin media without being exposed to cytokinin (FIG. 7B). Without being bound by any particular theory, a possible explanation for this result is the promotion of production of endogenous cytokinins by the GRF4-GIF1 chimera. This observation indicates that the GRF4-GIF1 chimera is able to promote embryogenesis and shoot regeneration without the addition of exogenous cytokinins, which can be used as a positive selection method to identify transgenic shoots without using antibiotic-based markers.

### Example 6—Constructing a miR396-Resistant GRF

The results from FIG. 6 show that increased expression of the GRF4-GIF1 chimera improved regeneration efficiency from wheat leaf explants, which indicates the potential of this method for different plant species and tissues. However, in some plant species or in particular tissues of some species, high levels of miR396 can cleave the GRF RNA and may limit the expression of high levels of the GRF protein or the GRF-GIF chimera.

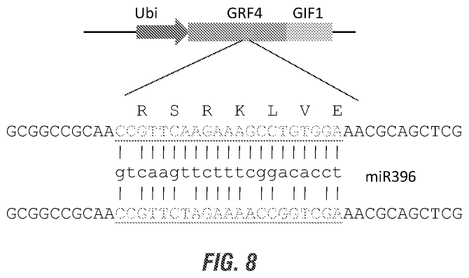

To avoid this potential problem, we generated a modified GRF gene (alone and in the GRF-GIF chimera, SEQ ID NO:34) which has silent mutations in the miR396 target site that render them less sensitive to the repression by miR396 (FIG. 8). Mutations in the miR396 binding site in Arabidopsis GRF3 (SEQ ID NO:19) or rice GRF4 (SEQ ID NO:17) have been shown to result in higher GRF activity and increased organ size (Debernardi et al., 2014; Beltramino et al., 2018; Duan et al., 2015; Hu et al., 2015; Che et al., 2015; Sun et al. 2016; Li et al. 2018).

### Example 7—Regulation of Expression Using an Inducible System

In wheat, we did not observe deleterious effects of the GRF4-GIF1 chimera on the plant phenotype, likely because the presence of endogenous miR396 was sufficient to cleave any excess GRF4 generated by expression of the transgene. However, it is possible that in other species the presence of a GRF-GIF chimera could affect the phenotype in undesirable ways. These effects might be more significant when using a GRF variants with mutations in the miR396 target site that render them less sensitive to the repression by miR396.

The ability to control the expression and/or activity of this chimera in plants by using an inducible system could address this possibility. Debernardi and Palatnik (patent application WO 2016/098027 A1) have demonstrated that in Arabidopsis the activity of a similar GRF-GIF chimera can be controlled exogenously by cloning a rat Glucocorticoid Receptor (GR) in the middle of GRF-GIF. The new GRF-GR-GIF chimera is activated only in the presence of the synthetic hormone dexamethasone. Using this strategy, we generated GRF-GR-GIF chimeras for Vitis vinifera (SEQ ID NO:31) and wheat (SEQ ID NO:32 and SEQ ID NO:33), which can be selectively induced only in the transformation media, and will not be active later in the transformed T₀ plants or in subsequent generations, avoiding potential effects on plant development.

Additionally, the inducible system would allow the use of hyperactive versions of the GRF4-GIF1 chimera, while limiting potential effects on plant development. For example, the incorporation of synonymous mutations in the miR396 target site of the GRF eliminates the miR396-mediated posttranscriptional repression of the chimera, boosting its expression and probably its activity even in tissues where miR396 is expressed (e.g., leaves). Through the inducible system, the GRF4-GIF1 chimera could be overexpressed only during regeneration. Another option is the use of tissue specific promoters.

### Example 8—Methods Using Protein Delivery

It is possible to deliver proteins to plant cells. Targeted mutagenesis was obtained in many plant species by delivering CRISPR/Cas9 preassembled ribonucleoprotein complexes (RNPs) (Woo et al., 2015; Subburaj et al., 2016; Malnoy et al., 2016; Kim et al., 2017; Liang et al., 2017; Svitashev et al., 2016; Wolter et al., 2017). The methods previously described could be performed by delivering GRF, GIF or GRF-GIF chimeric proteins into plants cells. As an example, GRF, GIF or GRF-GIF chimeras could be delivered together with CRISPR/Cas9 RNPs to boost the regeneration of the edited plant cells. After protein delivery, the plant tissues could then be cultured in regeneration media.

### Example 9—Transformation with Additional Vectors

Once a plant is transformed with a GRF, GIF or GRF-GIF chimera and acquires high regeneration ability, cells from this plant or its progeny could be further used for transient or stable transformation with vectors other than the GRF, GIF or GRF-GIF chimera vectors. The plant would still have high regeneration ability. The plant cells could then be cultured in regeneration media with either normal or reduced levels of cytokinins.

### Example 10—Transformation with Other GRF and/or GIF Sequences

The methods previously described could be performed with other GRF genes or related nucleotide sequences, e.g., in which the encoded amino acid sequences of QLQ and WRC domains of the GRF proteins are at least about 70%, about 80%, about 90%, or about 95% identical to the sequences of the QLQ and WRC domains of wheat proteins TaGRF1 (SEQ ID NO:9), TaGRF2 (SEQ ID NO:10), TaGRF3 (SEQ ID NO:11), TaGRF4 (SEQ ID NO:12), or TaGRF5 (SEQ ID NO:13), or to Arabidopsis proteins AtGRF3 (SEQ ID NO:19), AtGRF4 (SEQ ID NO:20), AtGRF5 (SEQ ID NO:21), or AtGRF6 (SEQ ID NO:22).

The methods may also be performed using a GRF gene in which the encoded amino acid sequence of the QLQ and WRC domains is at least about 80% similar, about 90% similar, or about 95% similar to the sequences provided in the preceding paragraph.

The percent identity and similarity are calculated by BLASTP using only the concatenated QLQ (underlined below) and WRC domains (in bold below), in the protein sequences presented in this disclosure. An example of a concatenated QLQ-WRC domain (SEQ ID NO: 96) is presented below for wheat TaGRF4 (for which the complete protein sequence is SEQ ID NO:12):

>TaGRF4 TraesCS6A01G269600
PFTAAQYEELEHQALIYKYLVAGVSVPPDLVLPIRDPEPGRCRRTD
GKKWRCAKEAASDSKYCERHMHRGRNRSRKPVE

Pairwise comparison of the concatenated QLQ-WRC domains among the five wheat proteins are presented in Table 1. All pairwise comparisons are higher than 70% identical and 80% similar, confirming their similarity and potential overlapping functions.

As an additional example of the conservation of these proteins, we included the comparison between the QLQ-WRC domains from the wheat GRF proteins and the corresponding domains in the orthologous rice (Oryza sativa) proteins. All pairwise comparisons show >70% identity and >80% similar (Table 2). These criteria exclude distantly related GRFs, such as OsGRF10 (LOC_Os02g45570.1), OsGRF11 (LOC_Os07g28430.1), and OsGRF12 (LOC_Os04g48510.1).

As a reference for dicot species, we compared the concatenated QLQ-WRC domains from the closest Arabidopsis GRF protein sequences AtGRF3 (SEQ ID NO:19), AtGRF4 (SEQ ID NO:20), AtGRF5 (SEQ ID NO:21), and AtGRF6 (SEQ ID NO:22) (Table 3). Table 3 shows that these Arabidopsis proteins are at least 70% identical or 80% similar to at least one of the wheat GRF proteins. The relatively high level of similarity found between the critical domains of this group of proteins in highly divergent dicot and monocot plants confirms the conservation of these proteins and the potential overlap in their functions. This potential is also supported by the observation that higher activity of Arabidopsis GRF3 (SEQ ID NO:19) or rice GRF4 (SEQ ID NO:17) due to mutations in the site recognized by miR396 increase seed size in these two distantly related species (Beltramino et al., 2018; Duan et al., 2015; Hu et al., 2015; Che et al., 2015; Sun et al. 2016; Li et al. 2018).

TABLE 1
Comparison among five wheat (Triticum aestivum) paralogous
proteins (TaGRF) included in this patent. The percent identity
(first number) and percent similarity (second number) of concatenated
QLQ-WRC conserved domains were obtained by BLAST P.

| % ID/% Sim | | | | |
|---|---|---|---|---|
| QLQ-WRC | TaGRF4 | TaGRF3 | TaGRF5 | TaGRF1 |
| TaGRF3 | 94/96 | — | — | — |
| TaGRF5 | 86/91 | 90/92 | — | — |
| TaGRF1 | 80/92 | 82/92 | 82/91 | — |
| TaGRF2 | 78/91 | 77/89 | 75/88 | 82/94 |

TABLE 2
Comparison between five wheat TaGRF proteins included in
this patent (sequences are available at the end of the document)
and the closest rice orthologs (Oryza sativa). The percent
identity and percent similarity of concatenated QLQ-WRC
conserved domains were obtained by BLAST P.

| % ID/% Sim | | | | | |
|---|---|---|---|---|---|
| QLQ-WRC | TaGRF4 | TaGRF3 | TaGRF5 | TaGRF1 | TaGRF2 |
| OsGRF4 | 95/96 | — | — | — | — |
| OsGRF3 | — | 97/97 | — | — | — |
| OsGRF5 | — | — | 96/97 | — | — |
| OsGRF1 | — | — | — | 96/98 | — |
| OsGRF2 | — | — | — | — | 87/96 |

TABLE 3
Comparison between five wheat TaGRF proteins included in this
patent and the closest Arabidopsis thaliana homologs (AtGRF).
The percent identity and percent similarity of concatenated
QLQ-WRC conserved domains were obtained by BLAST P. Sequences
are available at the end of this document.

| % ID/% Sim | | | | | |
|---|---|---|---|---|---|
| QLQ-WRC | TaGRF4 | TaGRF3 | TaGRF5 | TaGRF1 | TaGRF2 |
| AtGRF3 | 72/89 | 74/85 | 73/83 | 71/85 | 72/87 |
| AtGRF4 | 69/89 | 71/87 | 70/83 | 67/84 | 71/86 |
| AtGRF5 | 75/88 | 76/88 | 76/89 | 79/91 | 72/86 |
| AtGRF6 | 72/84 | 76/84 | 76/84 | 73/88 | 71/83 |

Likewise, the methods of this disclosure could be performed with other GIF genes or related nucleotide sequences, e.g., in which the encoded amino acid sequences of SNH domains of the GIF proteins are at least about 70% identical, about 80%, about 90%, or about 95% to the sequences of the SNH domains of wheat protein TaGIF1 (SEQ ID NO:27), TaGIF2 (SEQ ID NO:28), or TaGIF3 (SEQ ID NO:29), or to the Arabidopsis proteins AtGIF1 (SEQ ID NO:24), AtGIF2 (SEQ ID NO:25), or AtGIF3 (SEQ ID NO:26).

The methods may also be performed using a GIF gene in which the encoded amino acid sequence of the SNH domain is at least about 80% similar, about 90% similar, or about 95% similar to the sequences provided in the preceding paragraph.

A mutation in Arabidopsis GIF1, also known as ANGUSTIFOLIA3 (AN3), causes smaller, narrower leaves and petals, with a reduced number of cells, similar to the phenotypes seen in multiple grf mutants (Kim and Kende 2004; Horiguchi et al. 2005). Single gift and gif3 mutants are similar to wild type plants, suggesting that GIF1 has a larger impact on leaf development than other members of the GIF family (Lee et al. 2009). However, the phenotypes of double mutants gif1,2 or gif1,3 and of the triple mutant gif1,2,3 are more severe, indicating that GIF genes have redundant functions (Lee et al. 2009). In addition, ectopic overexpression of all three GIFs can rescue a mutation in GIF1 (Lee et al. 2009).

Based on the overlapping GIF functions described above, we predict that different GIF paralogs in wheat can have similar effects on the improvement of regeneration efficiency of transgenic plants either alone or as protein chimeras with different GRFs. Therefore, we include in this application Arabidopsis proteins AtGIF1 (SEQ ID NO:24), AtGIF2 (SEQ ID NO:25), AtGIF3 (SEQ ID NO:26) as well as the three closest wheat homologous proteins TaGIF1 (SEQ ID NO:27), TaGIF2 (SEQ ID NO:28), and TaGIF3 (SEQ ID NO:29), and any other plant protein that shows at least 70% identity or 80% similarity for the conserved SNH domain highlighted in the protein sequences described at the end of this document. Comparisons among the SNH domains of the three wheat GIF proteins showed identities of at least 70% and similarities of at least 89% (Table 4).

TABLE 4
Comparison among the three wheat (Triticum aestivum)
paralogous proteins (TaGIF) included in this patent.
The percent identity and percent similarity of the
conserved SNH domain were obtained by BLAST P.

| % ID/% Sim SNH | TaGIF2 | TaGIF3 |
|---|---|---|
| TaGIF1 | 75/92 | 70/89 |
| TaGIF2 | — | 95/98 |

When the wheat SNH domains were compared with the corresponding SNH domains from Arabidopsis, identities varied between 66% and 91% and similarities between 89 and 96% (Table 5). Each Arabidopsis SNH domain was at least 70% identical or 80% similar to at least one of the three wheat SNH domains (Table 5).

TABLE 5
Comparison between the three wheat TaGIF proteins included in
this patent (sequences above) and the closest Arabidopsis thaliana
homologs (AtGIF). The percent identity and percent similarity
of the conserved SNH domain were obtained by BLAST P.

| % ID/% Sim SNH | TaGIF1 | TaGIF2 | TaGIF3 |
|---|---|---|---|
| AtGIF1 | 73/89 | 66/89 | 65/89 |
| AtGIF2 | 71/90 | 89/96 | 84/89 |
| AtGIF3 | 72/90 | 91/96 | 83/91 |

### Example 11—Use in Combination with Genome Editing

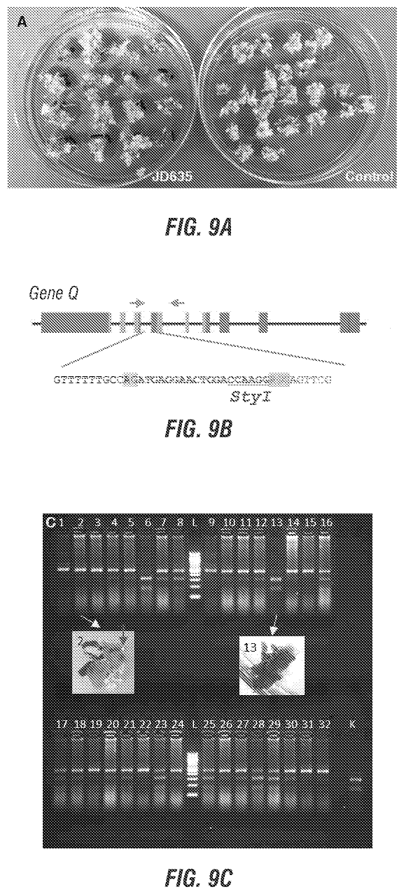

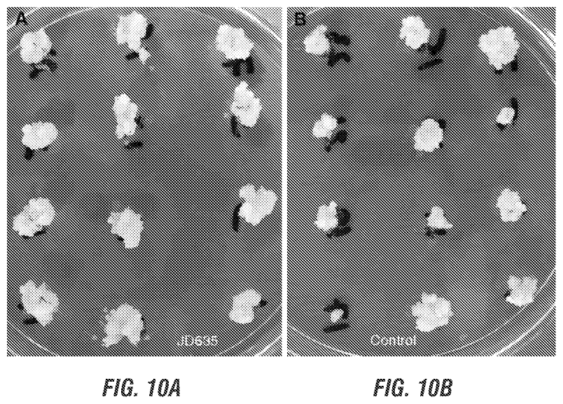

The methods provided in this disclosure may be ideal for use in combination with genome editing since the transgene can be segregated-out after the editing events are completed. This eliminates the risk that the GRF-GIF chimera would negatively affect the plant phenotype. The high transformation efficiency of the disclosed methods could expand the list of plant varieties that would benefit from CRISPR-screens to test panels of mutants in the regulatory and coding regions of important agronomic genes. We have confirmed high regeneration efficiency (FIG. 9A) of embryos transformed with vector JD635 (GRF4-GIF1-gRNA-GeneQ) including the GRF4-GIF1 chimera, Cas9 and a gRNA targeting Gene Q (FIG. 9B). We tested 32 callus from this transformation and confirmed editing of the target gene in 30 of them (FIG. 9C).

### Example 12—Selection of Transgenic Plants in Absence of Selectable Markers

The methods disclosed herein may be used to avoid the use of selectable markers, which are objected to by people opposed to the use of transgenic plants. The ability of the GRF4-GIF1 chimera to regenerate in the absence of cytokinins demonstrates that transgenic cells can be selected from non-transgenic ones by simply eliminating or greatly reducing the concentration of cytokinins. FIG. 10 shows calluses from the same JD635 construct presented in FIG. 9 (GRF4-GIF1-Cas9-gRNA-GeneQ) grown in a medium without hygromycin and with one-tenth of the normal cytokinin concentration. Multiple regenerating shoots were observed in the presence of JD635 (FIG. 10A, green arrows) and no regeneration was observed in the control (FIG. 10B) transformed with a DsRed (without the GRF4-GIF1 chimera).

Materials and Methods for Examples 1 to 12.

Vectors. All cloning PCRs were performed with Phusion High-Fidelity DNA Polymerase (NEB). Cloning reactions were done using the Gateway Cloning technology (Invitrogen). RNA was extracted from spikes using the Spectrum Plant Total RNA Kit (Sigma-Aldrich), treated with RQ1 RNase-free DNase (Promega), and then cDNA synthesis was carried out using SuperScript II Reverse Transcriptase (Invitrogen). To clone the coding region of wheat GRF4 encoding the protein described in (SEQ ID NO:12) and GIF1 encoding the protein described in (SEQ ID NO:27), PCRs were performed on the cDNA generated from Kronos spike. The sequence of the primers specific for GRF4 (Fw-GRF4a/Rev-GRF4a) and GIF1 (Fw-GIF1a/Rev-GIF1a) are indicated in Table 6.

The PCR fragments were first cloned in pDONR by a B/P gateway reaction and transformed into chemical competent E. coli DH5a. The sequence of the clones was confirmed by Sanger sequencing. The GRF4-GIF1 chimera was generated by overlapping PCR. In a first step, GRF4 and GIF1 coding sequences were amplified with primers FW-GRF4a/Rev-GRF4b and Fw-GIF1b/Rev-GIF1b from the pDONR-GRF4 and pDONR-GIF1 clones as template. The primer Rev-GRF4b generates a 3′ end that overlaps 12 nucleotides with the 5′ end of Fw-GIF1b. Those 12 nucleotides generate a bridge of four alanine amino acids between GRF4 and GIF1. Both PCR fragments were gel-purified and used as template in a second PCR with the primers Fw-GRF4/Rev-GIF1b. The sequence of the primers are indicated in Table 6. The resulting product was cloned in pDONR by B/P gateway reaction and transformed into chemical competent E. coli DH5a. The sequence of the vector was validated by Sanger sequencing. Next, the GRF4, the GIF1 and the chimera GRF4-GIF1 genes were cloned in the binary vector pLC41 by a L/R gateway reaction under the maize UBIQUITIN promoter and transformed into chemical competent E. coli DH5a. The resulting vectors for the individual genes pLC41:GRF4 (SEQ ID NO:1) and pLC41:GIF1 (SEQ ID NO:2), and for the chimera pLC41:GRF4-GIF1 (SEQ ID NO:5) were verified by restriction digestion and transformed by electroporation in Agrobacterium EHA105.

The Vitis vinifera VviGRF4-GIF1 chimera (DNA, SEQ ID NO:6), Protein, SEQ ID NO:7) was generated by gene synthesis. The DNA fragment was cloned into pDONR by B/P gateway reaction and transformed into chemical competent E. coli DH5a. The sequence of the vector was validated by Sanger sequencing. Next, the Vitis chimera GRF4-GIF1 was cloned in the binary vector pGWB14 binary vector by a L/R gateway reaction under the viral 35S promoter and transformed into chemical competent E. coli DH5a. The resulting vector pGWB14-VviGRF4-GIF1 (DNA, SEQ ID NO:8) was verified by restriction digestion and transformed by electroporation in agrobacterium EHA105. A dexamethasone inducible version VviGRF4-GR-GIF1 (SEQ ID NO:31) was generated by overlapping PCR.

The wheat dexamethasone inducible GRF4-GR-GIF1 chimera encoding protein (SEQ ID NO:32) was generated by overlapping PCR. In a first step, GRF4, GIF1 and a rat Glucocorticoid Receptor (GR) were amplified with primers FW-GRF4a/Rev-GRF4b, Fw-GIF1-GR/Rev-GIF1b, Fw-GR/Rev-GR. The primer Rev-GRF4b generates a 3′ end that overlap 12 nucleotides with the 5′ end of Fw-GR, and the Rev-GR generates a 3′ end that overlap 12 nucleotides with the 5′ end of Fw-GIF1-GR. Those 12 nucleotides generate a bridge of four alanine amino acids between GRF4 and GR and GR and GIF1. The three PCR fragments were gel-purified and used as template in a second PCR with the primers Fw-GRF4/Rev-GIF1b. The sequence of the primers are indicated in Table 6. The resulting product was cloned in pDONR by B/P gateway reaction and transformed into chemical competent E. coli DH5α. The sequence of the vector was validated by Sanger sequencing. Next, the chimera GRF4-GR-GIF1 was cloned in the binary vector pLC41 by a L/R gateway reaction under the maize UBIQUITIN promoter and transformed into chemical competent E. coli DH5α. The resulting vector pLC41:GRF4-GR-GIF1 (SEQ ID NO:33) was verified by restriction digestion and transformed by electroporation in Agrobacterium EHA105.

A miR396-resistant version of wheat GRF4-GIF1 (rGRF4-GIF1) (SEQ ID NO:34) was generated by overlapping PCR. In a first step, two PCR were performed with primers Fw-GRF4a/rGRF-Rev and rGRF-Fw/Rev-GIF1b using pLC41-GRF4-GIF1 clone as template. The primers rGRF-Fw and rGRF-Rev overlap in 17 nt and introduce silent mutations in the miR396 target site. Both PCR fragments were gel-purified and used as template in a second PCR with the primers Fw-GRF4/Rev-GIF1b. The sequence of the primers are indicated in Table 6. The resulting product was cloned in pDONR by B/P gateway reaction and transformed into chemical competent E. coli DH5α. The sequence of the vector was validated by Sanger sequencing. Next, the chimera rGRF4-GIF1 was cloned in the binary vector pLC41 by a L/R gateway reaction under the maize UBIQUITIN promoter and transformed into chemical competent E. coli DH5α. The resulting vector pLC41:GRF4-GIF1 was verified by restriction digestion and transformed by electroporation in Agrobacterium EHA105. A dexamethasone inducible version rGRF4-GR-GIF1 was generated by overlapping PCR.

To generate the JD635-GRF4-GIF1-Cas9- gRNA-GeneQ vector, a cassette including the maize UBIQUITIN promoter, the GRF4-GIF1 chimera and the Nos terminator was amplified by PCR. The PCR product was gel-purified and cloned by In-fusion (Takara Bio USA, Inc.) into the Asci site of the pYP25F binary vector containing a wheat codon optimized Cas9 (TaCas9) and transformed into chemical competent E. coli DH5α. The vector sequence was validated by Sanger sequencing. Next, a guide RNA construct targeting the coding region of Gene Q was cloned by Golden Gate reaction into two AarI sites of the vector and transformed into chemical competent E. coli DH5α. The resulting JD635-GRF4-GIF1-Cas9-gRNA-GeneQ vector (SEQ ID NO:35) was validated by Sanger sequencing and transformed by electroporation into Agrobacterium EHA105.

Control vectors: The JD518 vector, in pLC41 backbone, contains the GLOSSY promoter driving the expression of LhG4, which is an artificial transcription factor with no targets in wheat. The pOp:FT1 vector, in pLC41 backbone, contains the FT1 coding sequence cloned downstream of pOp, which is an artificial promoter. The pLC41-DsRed in pLC41 backbone, contains the DsRed coding sequence cloned downstream of maize UBIQUITIN promoter.

Wheat transformation. Transgenic wheat plants were initially generated using technology from Japan Tobacco (JT) technology. However, cytokinin concentrations were reduced in some of the later experiments. Two independent experiments were performed for each vector. In each experiment, at least 25 immature embryos from Kronos were transformed using Agrobacterium EHA105 with the pLC41 constructs described before. Selection of transgenic plants was conducted using hygromycin, except in the experiments with reduced cytokinins (FIG. 10) where no hygromycin or any other antibiotic was used. Regenerated plants were transferred to soil and transgene insertion was validated by DNA extraction and PCR. The primers used to genotype the T₀ plants are indicated in Table 6.

TABLE 6
Sequence of primers used in cloning and genotyping.

| | | |
|---|---|---|
| Cloning | Fw-GRF4a | GGGGacaagtttgtacaaaaaag |
| pDONOR | | cTGCCACCATGGCGATGCCGTAT |
| | | GCCTCT (SEQ ID NO: 106) |
| | Rev-GRF4a | GGGGACCACTTTGTACAAGAAAG |
| | | CTGAACGGTACATYTCGCCGGCG |
| | | AACAG (SEQ ID NO: 107) |
| | Fw-GIF1a | GGGGacaagtttgtacaaaaaag |
| | | cTGCCACCATGCAGCAGCAACAC |
| | | CTGATG (SEQ ID NO: 108) |
| | Rev-GIF1a | GGGGACCACTTTGTACAAGAAAG |
| | | CTGAACGGCTTCCTTCCTCCTCG |
| | | GT (SEQ ID NO: 109) |
| Over- | Fw-GRF4a | GGGGacaagtttgtacaaaaaag |
| lapping | | cTGCCACCATGGCGATGCCGTAT |
| PCR | | GCCTCT (SEQ ID NO: 110) |
| | Rev-GRF4b | GGCAGCGGCCGCGTACATYTCGC |
| | | CGGCGAACAG (SEQ ID |
| | | NO: 111) |
| | Fw-GIF1b | gcggccgctgccATGCAGCAGCA |
| | | ACACCTGATG (SEQ ID NO: |
| | | 112) |
| | Rev-GIF1b | GGGGACCACTTTGTACAAGAAAG |
| | | CTGAACGCTAGCTTCCTTCCTCC |
| | | TCGGT (SEQ ID NO: 113) |
| | Fw-GIF1- | aagcTgccgcGgccATGCAGCAG |
| | GR | CAACACCTG (SEQ ID NO: |
| | | 114) |
| | Fw-GR | TACgcggccgctgccgaagctcg |
| | | aaaaacaaag (SEQ ID NO: |
| | | 115) |
| | Rev-GR | CATGGCCGCGGCAGCTTTTTGAT |
| | | GAAACAGAAG (SEQ ID |
| | | NO: 116) |
| | rGRF-Fw | TCTAGAAAACCGGTCGAAACGCA |
| | | GCTCG (SEQ ID NO: 117) |
| | rGRF-Rev | TCGACCGGTTTTCTAGAACGGTT |
| | | GCGG (SEQ ID NO: 118) |
| Geno- | Fw-GRF4b | CCTCCGACTCCAAGTATTGC |
| typing | | (SEQ ID NO: 104) |
| | Rev-GIF1c | ATCATCAGGTTGGACGGGTA |
| | | (SEQ ID NO: 105) |

Plant growth conditions. Transgenics plants were grown in PGR15 growth chambers (Conviron) adjusted to 16 h of light (22° C.) and 8 h of darkness (18° C.). Intensity of the sodium halide lights measured at plant heads height was (˜260 μM m⁻²s⁻¹).

Phylogenetic analysis. The amino acid sequence of the GRF and GIF genes from Arabidopsis and rice were obtained from phytozome (phytozome.jgi.doe.gov/pz/portal.html). The amino acid sequences of wheat GRF and GIF genes were obtained from the wheat genome RefSeq v1.0. The phylogenetic analysis was performed using MEGA6.

### Example 13—Transformation of Wheat Using Single GRF4 or GIF1 Genes

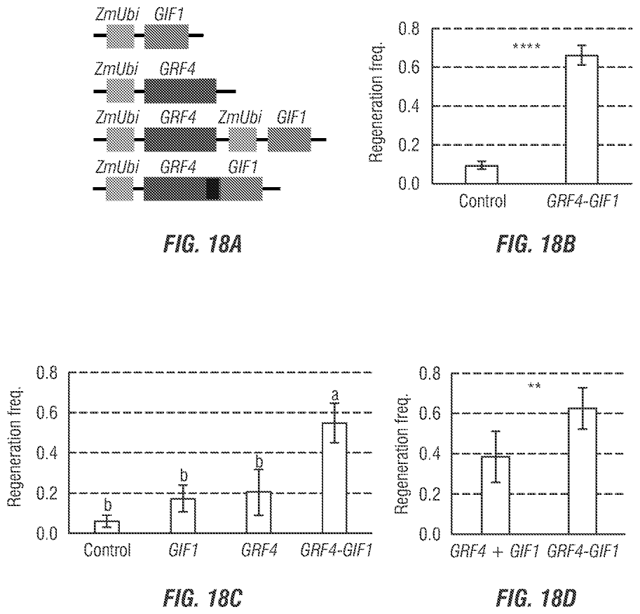

Wheat transformation experiments were performed using GRF4-GIF1 chimera (SEQ ID NO:5), GRF4 (SEQ ID NO:1) or GIF1 (SEQ ID NO:2) alone or within the same construct (FIG. 18A). Across 14 experiments (Table 7), the regeneration efficiency of the GRF4-GIF1 chimera (65.8±5.3%) was >7-fold higher than the empty vector control (8.7±2.0%, P<0.0001, FIG. 18B). In five separate transformation experiments (Table 7), we observed significantly lower regeneration efficiencies in embryos transformed with the GRF4 gene alone (20.4±11.4%) or the GIF1 gene alone (17.2±6.6%) relative to the GRF4-GIF1 chimera (54.6±9.8%, Tukey P<0.05, FIG. 18C). The regeneration efficiency of the calli transformed with the individual genes was approximately 3-fold higher than the control (6.0±3.0%) but the differences were not significant in the Tukey test (FIG. 18C).

We then compared the effect on regeneration efficiency of having the GRF4 and GIF1 fused in a chimera or expressed separately within the same construct by individual Ubi promoters (not fused, SEQ ID NO: 36) (FIG. 18A, Table 7). In five different experiments, the average regeneration efficiency of the separate GRF4 and GIF1 genes (38.6±12.9%) was significantly lower (P<0.0144) than the regeneration efficiency with the GRF4-GIF1 chimera (62.6±10.3%, FIG. 18D). This result demonstrated that the forced proximity of the two proteins in the chimera increased its ability to induce regeneration.

### Example 14—Wheat Transformation with Chimeras Including Other GRF and/or GIF Sequence Combinations

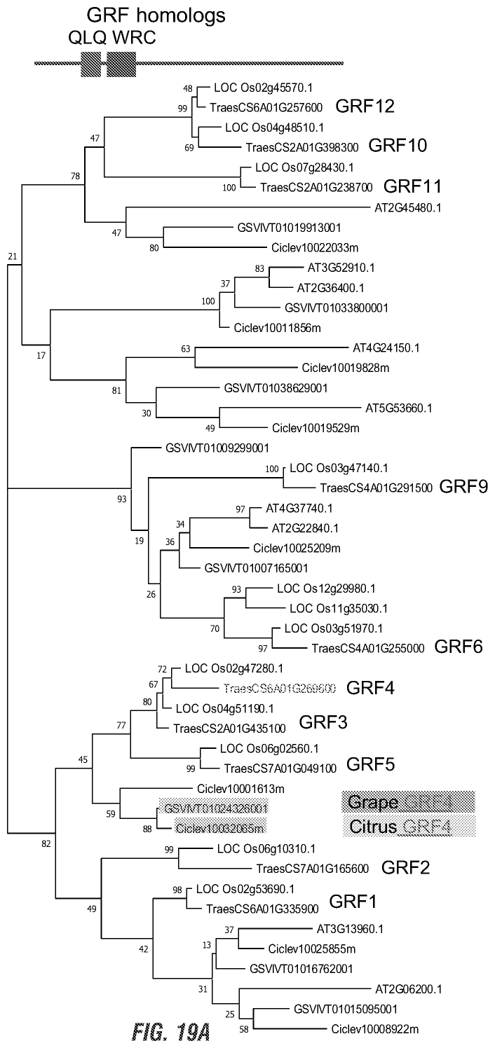

The GRF and GIF gene families contain several members (FIG. 19A, B), which display overlapping functions and redundancies. Therefore, we expected that other GRFs and/or GIFs besides GRF4 and GIF1 could also promote regeneration.

We tested GRF genes from different clades (FIG. 19A), including GRF5 (SEQ ID NO:13, 37) that is in the same clade as GRF4, GRF1 (SEQ ID NO:9, 38) that is in the closest clade (which includes Arabidopsis GRF5, SEQ ID NO: 21), and GRF9 (SEQ ID NO:39) that is in a more distal clade. Pairwise comparison of concatenated QLQ-WRC domains among eight wheat proteins, including the four tested in this patent, are presented in Table 8. All pairwise comparisons for GRF1-GRF6 and GRF9 are >70% identical or >80% similar, confirming their similarity and potential overlapping functions.

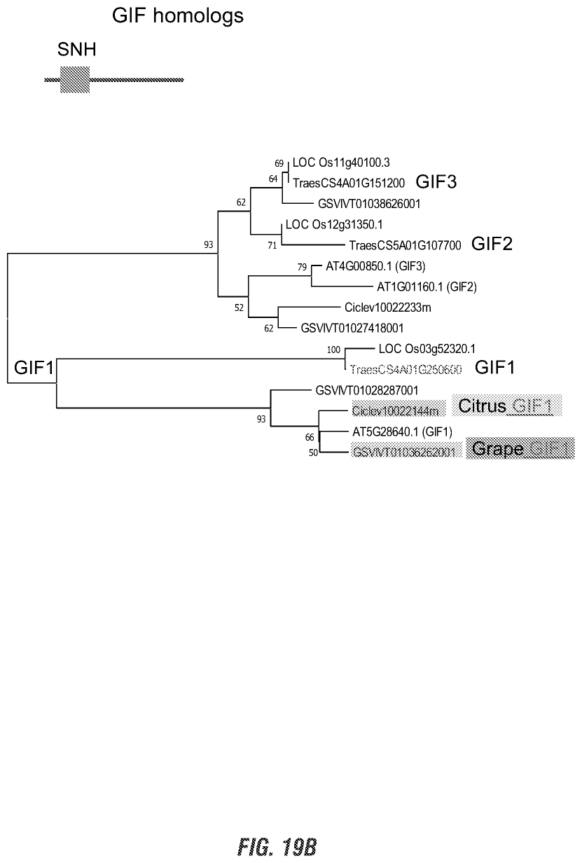

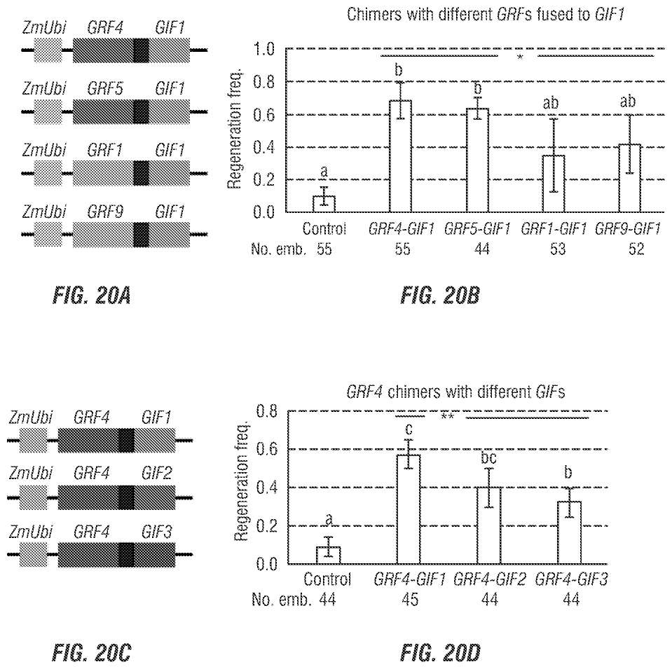

We generated chimeras for all the selected GRFs with GIF1 (FIG. 20A) (SEQ ID NO: 40-42) and tested transformation frequencies in Kronos. The regeneration efficiency induced by chimeras including the closely related GRF4 and GRF5 genes fused with GIF1, was significantly higher than the regeneration observed for chimeras including the more distantly related GRF1 and GRF9 genes fused with GIF1 (contrast P=0.0368, FIG. 20B). Interestingly, chimeras including GRF1 and GRF9 showed higher regeneration frequencies than the control, but the differences were not significant (Table 7). Therefore, GRF from different clades can promote regeneration, but GRFs from the GRF4-clade appear to have higher regeneration activity.

Next, we compared the activity of the three wheat GIF genes included in the phylogenetic tree (FIG. 19B) by generating chimeras with GRF4 (FIG. 20C). Like GIF1, chimeras including GIF2 (SEQ ID NO: 28, 43, 45) and GIF3 (SEQ ID NO: 29, 44, 46) also increased regeneration frequency compared to calli transformed with the empty vector (FIG. 20D, Table 7). However, the GRF4-GIF1 combination resulted in higher regeneration efficiency than the GRF4-GIF2 and GRF4-GIF3 combinations (contrast P=0.0046), and all three chimeras showed higher regeneration efficiency than the control (Tukey test P<0.05).

### Example 15. GRF4-GIF1 Improves Transformation of Wheat and Triticale Cultivars with Low Regeneration Efficiency or Recalcitrant

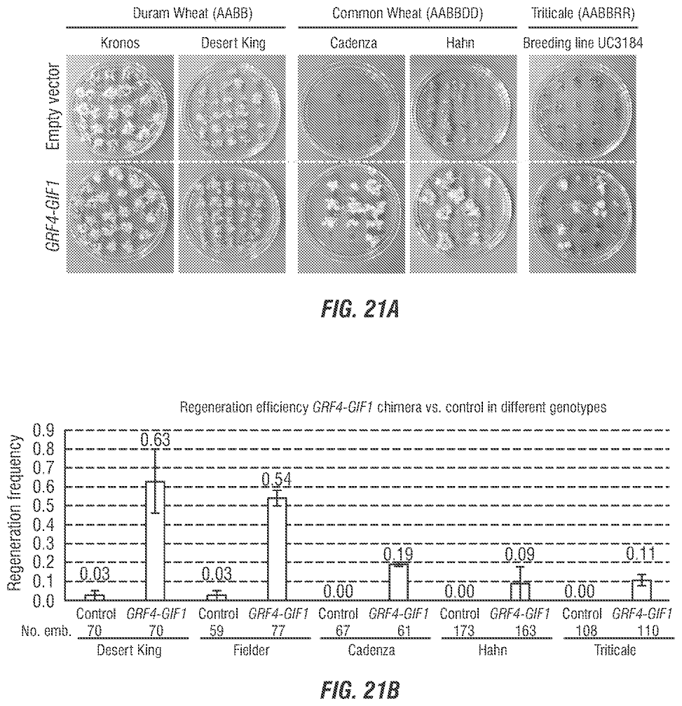

We then tested the potential of the GRF4-GIF1 chimera to generate transgenic plants from commercial durum, bread wheat and Triticale lines that were previously recalcitrant to Agrobacterium-mediated or had low regeneration efficiency at the UCD Plant Transformation Facility (FIG. 21). With the GRF4-GIF1 chimera we observed a 20 to 25-fold increase in regeneration frequencies in tetraploid wheat elite line Desert King (63.0±17.0% vs. 2.5±2.5%) and hexaploid wheat Fielder (54.0±4.0% vs. 2.5±2.5%) relative to the control. For the hexaploid wheat varieties Hahn and Cadenza and Triticale breeding line UC3190, for which we were not able to generate transgenic plants before, we observed regeneration frequencies of 9 to 19% with the GRF4-GIF1 chimera versus 0% with the control (FIG. 21 and Table 9A, B).

### Example 16. Wheat GRF4-GIF1 Improves Transformation of Rice Variety Kitaake

We also tested the wheat GRF4-GIF1 chimera in the rice variety Kitaake. In two independent transformation experiments, we observed a 2 to 3-fold increase in rice regeneration efficiency in the calli transformed with the wheat GRF4-GIF1 chimera (average 40.1±5.4%) compared with those transformed with the control vectors (17.6±5.9%, Table 9C). These results suggest that the wheat GRF4-GIF1 chimera is effective in enhancing regeneration in another agronomically important monocotyledonous species.

### Example 17—Regulation of Expression Using an Inducible System

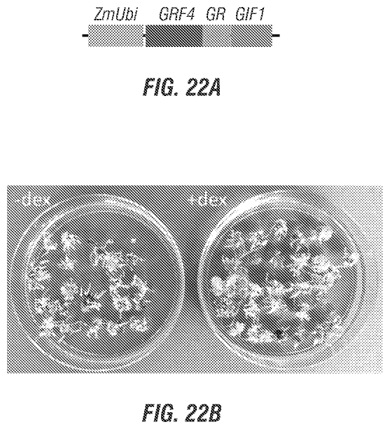

We generated an inducible version of the wheat GRF4-GIF1 chimera by cloning a rat Glucocorticoid Receptor (GR) in the middle of GRF4-GIF1 (SEQ ID NO:32, 33) (FIG. 22A). This chimera is activated only in the presence of the synthetic hormone dexamethasone (Dex). Immature embryos from Kronos were transformed using this vector, and then plated in regular transformation media or in media supplemented with 10 μM DEX. In the absence of DEX we recovered 1 shoot out of 23 embryos, while in the presence of DEX 4 shoots out of 24 embryos were recovered (FIG. 22B). This result indicated that a GRF-GR-GIF chimera is functional and can be controlled exogenously by the addition of DEX.

### Example 18—Regeneration of Transgenic Plants without Exogenous Cytokinins

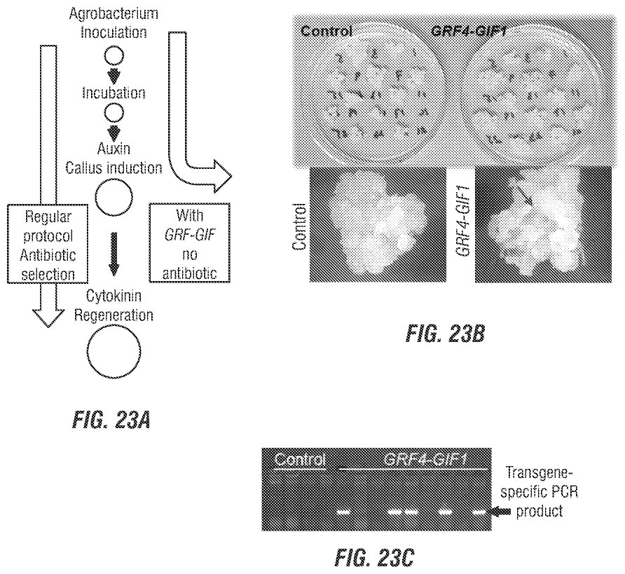

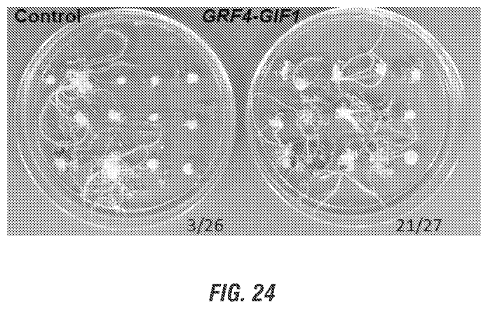

In many plant transformation systems cytokinins are required to regenerate shoots (FIG. 23A). Interestingly, we observed that embryos inoculated with Agrobacterium transformed with the wheat GRF4-GIF1 chimera were able to rapidly regenerate green shoots in auxin media without cytokinin (FIG. 23B). We then tested the regeneration efficiency of immature embryos from stable GRF4-GIF1 transgenics (n=27) and non-transgenic (n=26) T₁ sister lines in the absence of cytokinin and hygromycin. Under these conditions, the regeneration efficiency of the GRF4-GIF1 transgenic plants (77.8%) was significantly higher than the non-transgenic sister lines (11.5%, FIG. 24). These results indicated that the GRF4-GIF1 chimera can promote embryogenesis and shoot regeneration in wheat without the addition of exogenous cytokinin.

Based on the previous results, we developed a protocol to select transgenic shoots in auxin media without using antibiotic-based markers. In three experiment we recovered 40 shoots using a GRF4-GIF1 marker-free vector (SEQ ID NO:47) and 15 for the empty vector. Genotyping revealed that 10 out of the 40 (25%) GRF4-GIF1 shoots were transgenic, while none of the control was positive (FIG. 23C). These high-regenerating transgenic plants overexpressing the GRF4-GIF1 chimera without selection markers can be used for future transformation experiments to incorporate other genes using selectable markers. This strategy generates separate insertion sites for the GRF4-GIF1 and the second transgene, which facilitates the segregation of the GRF4-GIF1 insertion in the next generation.

### Example 19. GRF4-GIF1 Accelerates Wheat Transformation

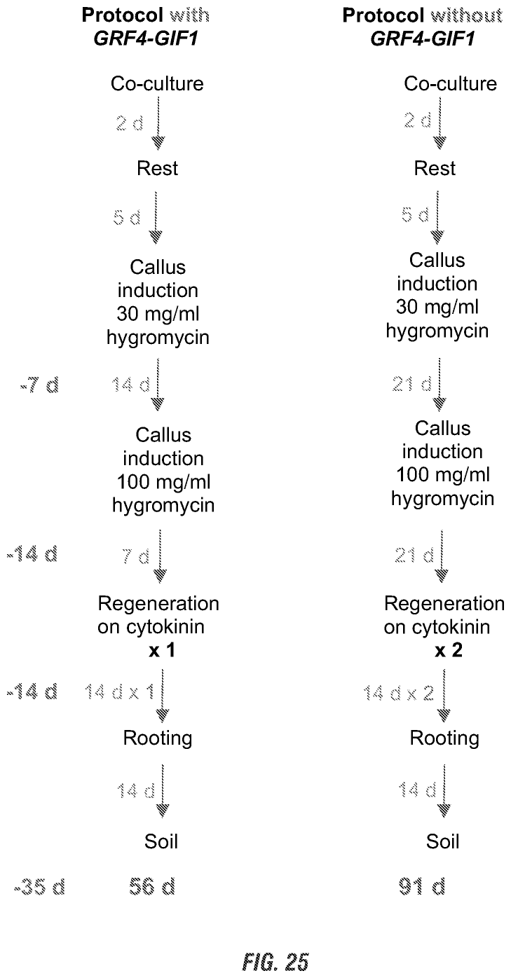

The wheat GRF4-GIF1 chimera accelerates the regeneration process, which allowed us to develop a faster wheat transformation protocol that takes 56 days instead of the 91 days required for all the wheat experiments presented in this manuscript (FIG. 25).

### Example 20. GRF-GIF Improves Transformation of Dicot Species

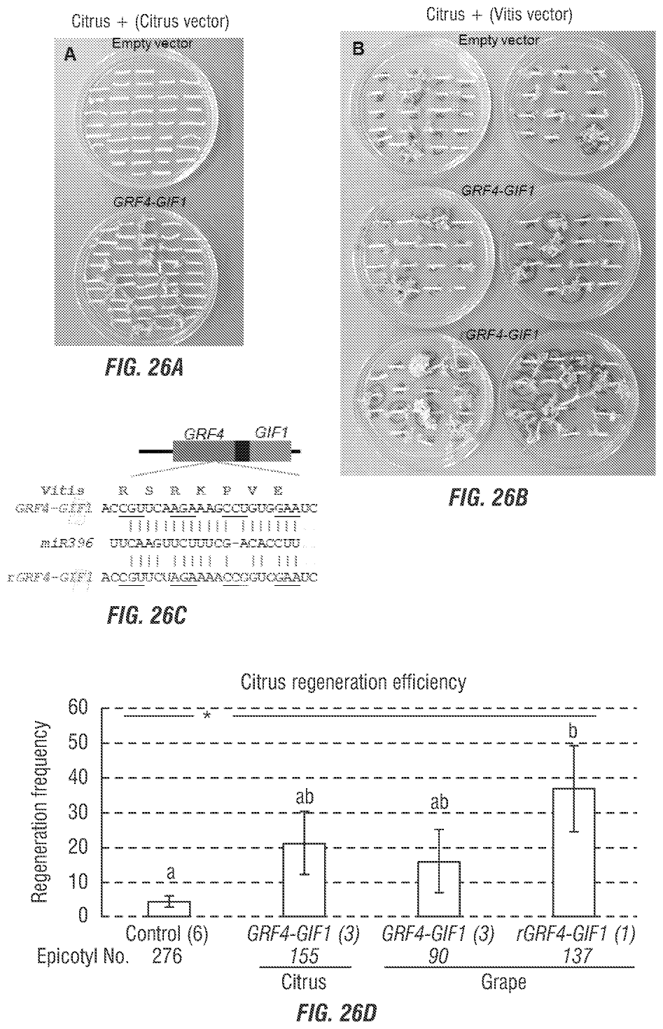

We performed a series of Citrus transformation experiments to test the effect of the GRF-GIF technology in a dicot crop with limited regeneration efficiency. We generated heterologous citrus (SEQ ID NO:48) and grape (SEQ ID NO: 6-8) GRF4-GIF1 chimeras using the closest homologs to wheat GRF4 (citrus Ciclev10032065m and grape GSVIVT01024326001), and GIF1 (citrus Ciclev10022144m and grape GSVIVT01036262001), in both species (FIGS. 19A and B). In three independent transformation experiments in the citron rootstock Carrizo, epicotyls were transformed with the citrus and the grape GRF4-GIF1 chimeras. Epicotyls transformed with the citrus GRF4-GIF1 chimera showed a 4.8-fold increase in regeneration frequency relative to those transformed with the empty vector control (FIG. 26A and Table 10). The heterologous grape GRF4-GIF1 chimera produced similar increases in citrus regeneration efficiency as the citrus chimera (FIG. 26B and Table 10).

We also tested the effect of a miR396-resistant grape GRF4-GIF1 version (henceforth, rGRF4-GIF1, SEQ ID NO:50), in which we introduced silent mutations in the GRF binding site for miR396 to avoid cleavage (FIG. 26B-C). In three independent experiments, we observed that the grape rGRF4-GIF1 chimera produced the highest frequency of transgenic events (8.4-fold increase compared to the control, P<0.05). A statistical analysis comparing the control versus the three combined GRF-GIF constructs was also significant (P=0.0153, FIG. 26D and Table 10). In spite of its higher-regeneration frequency, the rGRF-GIF construct would require additional optimization (e.g. an inducible system) because some of the transgenic events produced large calli that were unable to generate shoots (FIG. 26B).

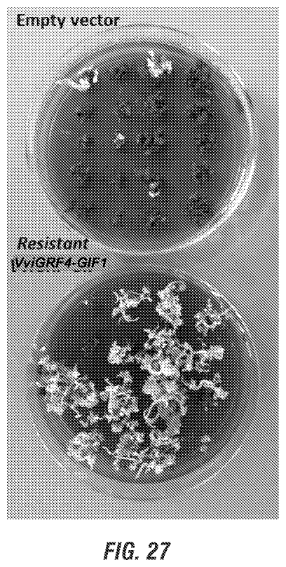

We tested the grape rGRF41-GIF1 chimera in grape transformation. The grape transformation process is slow and very laborious requiring 8 to 12 months for generating transgenic plant lines. Pro-embryogenic callus generated from anther filaments of immature flowers were inoculated with agrobacterium harboring the empty vector and the rGRF4-GIF1 vector. After 6 month of culture we observed that 87% of calli transformed with rGRF4-GIF1 regenerated shoots, while only 12% of the control calli produce shoots (FIG. 27). Interestingly, in contrast to citrus rGRF4-GIF1 transgenics, all grape rGRF4-GIF1 developed shoots that produced leaves.

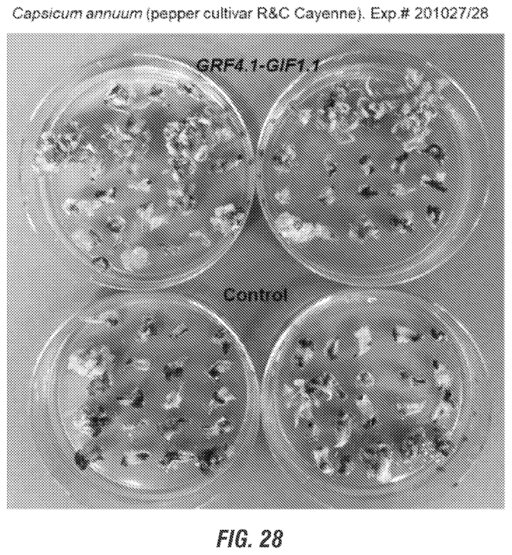

We then tested the GRF-GIF technology in pepper (Capsicum annuum) transformation using the cultivar “R&C cayenne”. In Capsicum annuum, there are two close paralogs of GRF4, designated here as GRF4.1 and GRF4.2, and two close paralogs of GIF1, designated here as GIF1.1 and GIF1.2. We used the GRF4.1 (LOC107869915) and GIF1.1 (LOC107870303) paralogs in the construction of the GRF4.1-GIF1.1 chimera (SEQ ID NO: 138-139), which was cloned into the binary vector pEarleyGate100 that carries BAR selection. For the transformation experiment, we used the basal portion of the cotyledons proximal to the shoot apex, with the petiole still attached. The 40 cotyledon pieces transformed with the pepper GRF4.1-GIF1.1 chimera showed a 23.8% regeneration efficiency, which was 4.76-fold higher than the regeneration efficiency of cotyledon pieces transformed with the empty vector pEarleyGate100 (5.0% regeneration efficiency, Table 11, FIG. 28).

Methods for Examples 13 to 20.

Method 1. Vectors Used in the Transformation Experiments.

Wheat vectors. To generate the vector expressing both GRF4 and GRF1 but not fused (Ubi::GRF4-term Ubi::GIF1-term, SEQ ID NO: 36), the complete Ubi::GRF4-term cassette was amplified by PCR using pLC41:GRF4 (SEQ ID NO:1) as template with primers

Fw_HindIII gccactcagcaagctttgcagcgt (SEQ ID NO: 119) and

Rev-HindIII TCACGCTGCAAAGCTCTAATTCCCGATCTAGTAAC (SEQ ID NO: 120). We cloned the PCR fragment in pGEMT-easy and we sub-cloned the Ubi::GRF4-term fragment into the HindIII site of pLC41:GIF1 (SEQ ID NO:2).

To generate the different wheat GRF-GIF chimeras, the coding sequences of GRF1 (SEQ ID NO: 38), GRF5 (SEQ ID NO:37), GRF9 (SEQ ID NO: 39), GIF2 (SEQ ID NO: 43) and GIF3 (SEQ ID NO: 44) were obtained by gene synthesis. Then, the different chimeras (GRF1-GIF1, GRF5-GIF1, GRF9-GIF1, GRF4-GIF2, GRF4-GIF3) were generated by overlapping PCR following the same strategy described to generate GRF4-GIF1. We cloned all the chimeras in pLC41 vector by L/R reaction and verified the vectors by restriction digestion and transformed by electroporation in Agrobacterium strain EHA105.

We generated the citrus GRF4-GIF1 chimera (SEQ ID NO: 48) by gene synthesis and cloned in pDONR. After checking the sequence by Sanger sequencing, we cloned it by L/R reaction into pGWB14 binary vector under the viral 35S promoter and transformed it into chemical competent E. coli DH5α. We verified the resulting vector pGWB14-citrus GRF4-GIF1 (DNA, SEQ ID NO:49) by restriction digestion and transformed by electroporation in Agrobacterium EHA105.

We generated a miR396-resistant version of Vitis GRF4-GIF1 (rGRF4-GIF1) by overlapping PCR. Two PCR reactions were performed with primers

Fw-GRF
(SEQ ID NO: 121)
GGGGacaagtttgtacaaaaaagcTGCCACCATGAAGCAAAGCTTTGTGG
/rGRF-Rev
(SEQ ID NO: 122)
TCGACCGGTTTTCTAGAACGGTTGCGG
and
rGRF-Fw
(SEQ ID NO: 123)
TCTAGAAAACCGGTCGAATCACAAACTA
/Rev-GIF
(SEQ ID NO: 124)
GGGGACCACTTTGTACAAGAAAGCTGAACGTCAATTCCCATCTTCAGCA

using pGBW14-vitis GRF4-GIF1 clone as template (SEQ ID NO: 8). The primers rGRF-Fw and rGRF-Rev overlap in 17 nucleotides and introduce silent mutations in the miR396 target site (FIG. 26C). Both PCR fragments were gel-purified and used as template in a second PCR with the primers Fw-GRF/Rev-GIF. We cloned the resulting product in pDONR by B/P gateway reaction. Next, we cloned the chimera rGRF4-GIF1 in the binary vector pGWB14 by a L/R gateway reaction under the viral 35S promoter. The resulting vectors were transformed by electroporation in Agrobacterium strain EHA105.

We generated the pepper GRF4.1-GIF1.1 chimera (SEQ ID: NO: 140) by gene synthesis and cloned it into the binary vector pEarleyGate100 under the 35S promoter. pEarleyGate100 has a BAR selection marker. We transformed the construct, designated pTH1903, in Agrobacterium strain EHA105 by electroporation. As a control, we used the empty vector pEarleyGate100.

Plant Transformation

Method 2. Wheat transformation protocol. Wheat transformation followed previously published protocols. Briefly, we grew the different wheat and triticale cultivars in a growth chamber under long-day photoperiod (16 h of 380 μM m⁻²s⁻¹ light, 26° C. day and 18° C. night). We harvested immature grains from spikes approximately 2 weeks after anthesis, and surface sterilized for 1 minute in 70% ethanol followed by 10 minutes in 1.2% (v/v) sodium hypochlorite solution plus 5 μl tween. After surface sterilization, we washed the seeds three times with sterilized water and isolated immature embryos under stereoscopic microscope.

We centrifuged the isolated immature embryos in liquid medium and then inoculated with Agrobacterium. We transferred the embryos to co-cultivation medium with the scutellum-side up and incubated at 23° C. in the dark. After 2-3 days, we excised the embryo axis, and transferred them to callus induction medium without selection, where we incubated them at 25° C. in the dark. After 5 days, we transferred the embryos to selection medium with 30 mg/l of hygromycin and incubated them at 25° C. in the dark.

After 3 weeks, we transferred the calli to selection medium that contained 100 mg/l of hygromycin. After an additional 3 weeks, we transferred the proliferating tissue to regeneration medium containing 50 mg/l of hygromycin and incubated them at 25° C. under continuous light (30 μM m⁻²s⁻¹) for 2 weeks. We transferred the regenerated shoots into rooting medium contained 50 mg/l of hygromycin. Rooted plants were acclimated to soil by transferring them to a 1020 tray containing a 36 sheet inserts filled with Sunshine potting mix and covered with an 11×21×2 inch clear plastic dome for 10 days under 16 hour of 100 μM light and 26° C. More recently, we developed a shorter transformation protocol to generate GRF4-GIF1 transgenic wheat plants that is summarized in FIG. 25.

Method 3. Rice transformation protocol. Rice transformation followed previously published protocols (Ishida et al. 2015, Proc. 12^{th} Int. Wheat Genet. Symp. 167-173). Briefly, we selected fresh rice seeds, de-husked them and surface sterilized them in a rotating flask containing 20% (v/v) bleach for 30 min. Then, rinsed the seeds 3 times with sterile water. We placed about 25-50 seeds per plate on callus induction media (MSD, 1× Murashige and Skoog with vitamins medium containing 30 g/l sucrose, 2 mg/l 2,4-dichlorophenoxyacetic acid, 1.2% (w/v) agar, pH 5.6-5.8) without letting the embryo touch the media, wrapped plate with surgical tape end incubated under 16 h light/8 h dark at 28° C. After 10-14 d, we separated the callus from the rest of the germinating seed and transferred to fresh MSD agar plates for another 5 d before co-cultivation.

Agrobacterium culture: We prepared a glycerol freezer stock from a single bacterial colony isolated from a plate. We then inoculated 1 ml LB containing the appropriate antibiotics to maintain the Agrobacterium and the plasmid, and we incubated it overnight at 28° C. at 250 rpm. The following day we added 300 μl of the Agrobacterium culture to 20 ml TY (pH 5.5) containing the appropriate antibiotics and 200 μM acetosyringone. We incubated the culture 28° C. for in a shaking incubator set at 250 rpm until the culture reached an OD₆₀₀ between 0.1-0.2 (approximately 2-4 h).

Transformation and co-cultivation: We placed the calli in Agrobacterium suspension for 30 min and shook the suspension to ensure uniform access to the calli. After the shaking incubation, we dried the calli on sterile Whatman paper to remove excess bacterial suspension. We transferred the calli onto co-cultivation medium (MSD+S+AS, 1× Murashige and Skoog with vitamins medium containing 30 g/l sucrose, 5% sorbitol, 2 mg/l 2,4-dichlorophenoxyacetic acid, 200 μM acetosyringone, 1.6% (w/v) agar, pH 5.6-5.8) and incubated for 3 d in the dark at 22° C.

Selection: We transferred the co-cultivated calli to selection media (MSD+CH+PPM, 1× Murashige and Skoog with vitamins medium containing 30 g/l sucrose, 2 mg/l 2,4-dichlorophenoxyacetic acid, 400 mg/L carbenicillin, 200 mg/l timentin, 1 ml/l Plant Preservative Mixture, 80 mg/L hygromycin, 1.2% agar, pH 5.6-5.8) and incubated the plates under continuous light at 28° C. We subcultured these calli onto fresh selection media every 8-9 d.

Regeneration and Rooting: After 4-5 weeks on selection media, resistant micro-calli of approximately 2-5 mm wide started to appear. We picked these off the original callus and transferred them to Petri dishes with regeneration media (BN+S+CH, 1× Murashige and Skoog with vitamins medium containing 30 g/l sucrose, 5% sorbitol, 3 mg/l BAP, 0.5 mg/l NAA, 400 mg/l carbenicillin, 200 mg/l timentin, 1 ml/l Plant Preservative Mixture, 50 mg/l hygromycin, 1.6% (w/v) agar, pH 5.6-5.8), and incubated under continuous light at 28° C. We subculture these calli onto fresh regeneration media every 8-9 days. After 4-5 weeks, when calli started to turn green, the regenerated plants were transferred to rooting media (MS+H, 1× Murashige and Skoog with vitamins medium containing 50 mg/l hygromycin, 1.2% (w/v) agar, pH 5.6-5.) and incubated in 16 h light/8 h dark 28° C. When the roots were well developed, we transferred the plants to soil.

Method 4. Citrus transformation protocol. We placed seeds of Carrizo citrange rootstock in water to imbibe and then peel off the seed coats making sure not to remove the integument. We surface sterilized seeds in 0.6% (v/v) sodium hypochlorite solution plus 5-μl tween 20 by placing them in a 50 ml centrifuge tube and shaking at 100 rpms for 20 minutes. We rinse the seeds 3× in 150-200 ml of sterile distilled water. We placed seeds on agar solidified ½× Murashige and Skoog minimal organics medium (½×MSO) containing 15 g/l sucrose, 7 gm TC agar (pH 5.6-5.8), and push seeds slightly into the medium for more uniform germination. Incubate in the dark at 26° C.

Agrobacterium culture: We prepared a glycerol freezer stock from a single bacterial colony isolated from a plate. We then used 40 μl of the stock to inoculate 20 ml of MGL medium (pH 7.0) containing the appropriate antibiotics to maintain the Agrobacterium and the plasmid, and we incubated overnight at 28° C. at 250 rpm. The following day, we removed 5 ml of the overnight growth and transferred it to 15 ml of TY medium (pH 5.5) containing the appropriate antibiotics and 200 μM acetosyringone. We incubated the culture overnight at 28° C. at 250 rpm and then diluted the overnight culture grown in TY medium to an O.D 600 nm of 0.1 to 0.2.

Co-cultivation: We collected 2-5 week old etiolated epicotyls and place in a petri dish containing 10 ml of the Agrobacterium solution prepared above (0.1-0.2 OD 600). We cut submerged epicotyls into 0.5 cm sections and soak for 10 min. We transferred the epicotyl sections onto co-cultivation medium consisting of Murashige and Skoog minimal organics medium (MSO) modified with 30 g/l sucrose 3.0 mg/l BAP, 0.1 mg/l NAA, and 200-μM acetosyringone pH 5.6-5.8. Incubate at 23° C. in the dark.

Induction: After 2-3 days, we transferred the epicotyl pieces to induction medium consisting of MSO modified with 30 g/l sucrose, 3.0 mg/l BAP, 0.1 mg/l NAA, 400 mg/l carbenicillin, 150 mg/l timentin and 100 mg/l kanamycin sulfate, and incubated them in the dark. After 10 days, we subcultured the epicotyl sections to fresh medium of the same formulation and then subcultured them every 21 d. After the second 21-day cycle in the dark, we transferred cultures to light under a 30 μM light and a photoperiod of 16 h light 8 h dark. We continued to transfer every 21 d to fresh medium of the same media until organogenic shoot buds develop at the cut ends.

Elongation: Once shoots began to form, we transferred the developing shoots to elongation medium consisting of MSO modified with 30 g/l sucrose, 0.1 mg/l BA, 400 mg/l carbenicillin, 150 mg/l timentin, and 100 mg/l kanamycin sulfate. We incubated as above and subcultured the cultures every 21 d as needed until shoots elongated.

Rooting: Once a shoot reached 2-4 cm in height, we harvested the shoots and transferred them to rooting medium consisting of MSO modified with 30 g/l sucrose, 5 mg/l NAA, 250 mg/l cefotaxime, and 100 mg/l kanamycin. After three to five days, we transferred shoots to MSO modified with 30 g/l sucrose, 0.0 mg/l NAA, 400 mg/l carbenicillin and 100 mg/l kanamycin. Shoots started rooting in 14 days.

Method 5. Grape transformation protocol. For generating transgenic grape (Thompson Seedless), we transferred somatic embryos to 5 ml of liquid Lloyd and McCown WPM supplemented with 20 g/l sucrose, 1 g/l casein, 1 mM IVIES, 500 mg/l activated charcoal, 0.5 mg/l BAP, 0.1 mg/l NAA, 200 μM acetosyringone and 12.5 μl pluronic F68. We heat-shocked the embryos by placing the tube in a 45° C. bath for 10 min.

We added Agrobacterium to the solution to an OD of 0.1 to 0.2, and then transferred the embryos to an empty 100×20 mm petri dish containing a 7 mm Whatman filter paper disk. We wrapped the petri dish with parafilm, and incubated it in the dark at 23° C. After 2-3 days, we harvested the embryos and transferred them to WPM selection medium. We supplemented the medium with 20 g/l sucrose, 1 g/l casein, 1 mM IVIES, 500 mg/l activated charcoal, 0.5 mg/l BAP, 0.1 mg/l NAA, 400 mg/l carbenicillin, 150 mg/l timentin, 200 mg/l kanamycin (or 25 mg/l hygromycin), 50 g/sorbitol, 14 g/l agar and 4 ml plant preservative mixture (PPM).

We incubate the embryos in the dark at 26° C. and after 7 days, we subcultured the embryos to fresh medium of the same formulation. After 14 and 28 days, we subcultured the embryos to fresh medium. After an additional 14 days, we transferred the embryos to the same WPM supplemented medium but without sorbitol and with 8 g/l agar. We divided the embryos on the plate into independent clusters and incubated them under 16 h light at 26° C., with subcultures every two to three weeks. We continue subculturing every 14-21 days until plants form.

To harvest the germinating plants, we excised the root and placed the shoot on WPM supplemented with 30 g/l sucrose, 0.01 mg/l IBA, 150 mg/l timentin, 400 mg/l carbenicillin and 100 mg/l kanamycin (or 25 mg/l hygromycin). Once shoots rooted, we transferred them to a 2-inch pot containing moistened soil (1 part sunshine mix 2 part vermiculite) and placed the pot into a sealed Ziploc bag for 7 days. Incubate at 26° C. under 16 h of 100 μM m-2 s-1 light covering the flat with a plastic dome. After one week, we opened the Ziploc bag to reduce humidity. Once new growth was evident, we removed the plants from the Ziploc bag and place them under the dome for an additional 7 days to complete acclimatization.

Method 6. Pepper Transformation Protocol.

We surface sterilized Capsicum annuum cultivar “R&C cayenne” seeds in a 1.2% (v/v) sodium hypochlorite solution containing 5 ul tween 20. We agitated the seeds on an orbital shaker at 100 rpms for 20 m followed by one rinse in 150-200 ml of sterile distilled water. We transferred them to agar-solidified ½ strength Murashige and Skoog minimal organics medium (½×MSO) supplemented with 16 g/l glucose, 600 mg/l IVIES, 8 gm PhytoAgar (Plantmedia cat number 40100072-4) (pH 5.6-5.8). We incubated the seeds at 26° C. under a 16 h photoperiod and 30 μM m2 s-1 light.

After 10 d, we placed the seedlings in a solution of Agrobacterium tumefaciens (strain EHA 105) adjusted to an O.D. 600 of 0.1-0.2. While submersing the seedlings in the Agrobacterium solution, we removed ⅔ of the tip of the cotyledons and then remove the cotyledons from the seedling taking care to retain the petiole of the cotyledon but not the apical meristem. After 5 minutes, we transferred the explants into 100×20 mm petri dishes containing co-cultivation medium consisting of MSO medium modified with 16 g/l glucose, 600 mg/l IVIES, 10 mg/l benzlaminopurine (BAP), 1.0 mg/l indole-3-acetic acid (IAA), and 200 μM acetosyringone pH 5.6-5.8. We incubated the tissue in the dark at 23° C.

After 2-3 d, we transferred the cotyledon pieces to induction medium consisting of MSO medium supplemented with 16 g/l glucose, 600 mg/l MES, 10 mg/l BAP, 1.0 mg/l IAA, 300 mg/l timentin, 400 mg/l carbenicillin, 8 mg/l glufosinate (Sigma-Aldrich catalog number 45520). After 10 d, we sub-cultured the cotyledons to fresh medium and then subsequently every 21 d.

Once buds began to form, we transferred the developing buds to elongation medium. This medium consisted of Driver and Kuniyaki Walnut (DKW) medium, supplemented with 30 g/l glucose, 1.3 g/l ca gluconate, 2.0 mg/l meta-topolin, (MT) 10 mg/l gibberellic acid (GA3), 2 ml/l of a silver thiosulfate stock (STS) (1.0 ml of a 100 mg/ml stock of AgNO3 added to 1.0 ml of a 12 mM (95 mg/50 ml water stock), 300 mg/l timentin, 400 mg/l carbenicillin, 4 ml/l PPM and 8 mg/l glufosinate.

As shoots begin to develop from the buds, we transferred them to elongation medium consisting of DKW supplemented with 30 g/l glucose, 1.3 g/l ca gluconate, 0.5 mg/l MT, 10 mg/l GA3, 2.0 ml/l STS, 300 mg/l timentin, 400 mg/l carbenicillin, 4 ml/l PPM and 8 mg/l glufosinate. We incubated as above transferring to fresh medium every 14 days. We removed any developing callus at the base of the shoots at each subculture (FIG. 28).

After two consecutive, 14 d subcultures, we transferred elongating shoots to DKW medium supplemented with 30 g/l glucose, 1.3 g/l ca gluconate, 0.1 mg/l MT, 10 mg/l GA3, 1.0 ml/l STS, 300 mg/l timentin, 400 mg/l carbenicillin, 4 ml/l PPM and 8 mg/l glufosinate. When shoots reached a size of 2-3 cm, we transferred them to rooting medium consisting of DKW medium supplemented with 30 g/l sucrose, 0.1 mg/l NAA, 2 ml/l STS, 300 mg/l timentin 400 mg/l carbenicillin 4 ml/L PPM and 4 g/l glufosinate.

TABLE 7
Regeneration frequencies for different GRF-GIF combinations compared with
empty vector in tetraploid wheat Kronos (17 experiments). The number of embryos (n) per
genotype inoculated with each specific construct. Regeneration frequencies were estimated as the
number of calluses showing at least one regenerating shoot/total number of inoculated embryos.
This is a conservative estimate since calli generated from embryos transformed with the GRF4-
GIF1 construct usually generate multiple independently transformed shoots. The blue “x” indicate
the experiments included in the statistical analyses presented in the different figures. All
experiments in this Table were done with the regular 91 d protocol.

| | | | | | GRF4 | | | | | | | | | | | |
|---|---|---|---|---|---|---|---|---|---|---|---|---|---|---|---|---|
| | No inoc. | | GRF4- | FIG. | & | FIG. | | | FIG. | GRF4- | | FIG. | GRF5- | GRF1- | GRF9- | FIG |
| | embryos | pLC41 | GIF1 | 1B | GIF1 | 1D | GIF1 | GRF4 | 1C | GIF2 | GIF3 | 20D | GIF1 | | | 20B |
| Exp1-a | n = 25 | 0.04 | 0.90 | x | | | | | | | | | | | | |
| Exp1-b | n = 25 | 0.08 | 0.96 | x | | | | | | | | | | | | |
| Exp2 | n = 30 | | 0.27 | | 0.06 | x | | | | | | | | | | |
| Exp3 | n = 80 | | 0.91 | | 0.77 | x | | | | | | | | | | |
| Exp3b^{a} | n = 40 | | 0.60 | | 0.14 | x | | | | | | | | | | |
| Exp4 | n = 50 | 0.13 | 0.70 | x | 0.46 | x | 0.57 | | | | | | | | | |
| Exp6 | n = 20 | 0.20 | 0.65 | x | 0.50 | x | 0.35 | | | | | | | | | |
| Exp22 | n = 24 | 0.16 | 0.82 | x | | | 0.16 | 0.64 | x | | | | | | | |
| Exp25 | n = 15 | 0.00 | 0.70 | x | | | 0.40 | 0.06 | x | | | | | | | |
| Exp25b ^{a} | n = 15 | 0.00 | 0.35 | x | | | 0.00 | 0.00 | x | | | | | | | |
| Exp26 | n = 20 | 0.08 | 0.55 | x | | | 0.20 | 0.20 | x | | | | | | | |
| Exp26b ^{b} | n = 16 | 0.06 | 0.31 | x | | | 0.10 | 0.12 | x | | | | | | | |
| Exp12 | n = 10 | 0.00 | 0.50 | x | | | | | | 0.20 | 0.20 | x | | | | |
| Exp13 | n = 25 | 0.17 | 0.72 | x | | | | | | 0.50 | 0.46 | x | | | | |
| Exp24 | n = 10 | 0.10 | 0.50 | x | | | | | | 0.50 | 0.30 | x | | 0.10 | 0.30 | x |
| Exp17 | n = 20 | 0.00 | 0.67 | x | | | | | | | | | 0.57 | 0.16 | 0.19 | x |
| Exp18 | n = 24 | 0.20 | 0.88 | x | | | | | | | | | 0.70 | 0.79 | 0.76 | x |

TABLE 8
Comparison among the eight wheat (Triticum aestivum) paralogous proteins (TaGRF).
The percent identity (first number) and percent similarity (second number) of concatenated
QLQ-WRC conserved domains were obtained by BLAST P.

| % ID/% Sim | | | | | | | |
|---|---|---|---|---|---|---|---|
| QLQ-WRC | TaGRF4 | TaGRF3 | TaGRF5 | TaGRF1 | TaGRF2 | TaGRF9 | TaGRF6 |
| TaGRF3 | 94/96 | — | — | — | — | — | — |
| TaGRF5 | 86/91 | 90/92 | — | — | — | — | — |
| TaGRF1 | 80/92 | 82/92 | 82/91 | — | — | — | — |
| TaGRF2 | 78/91 | 77/89 | 75/88 | 82/94 | — | — | — |
| TaGRF9 | 70/83 | 72/82 | 73/82 | 71/84 | 68/83 | — | — |
| TaGRF6 | 72/87 | 73/83 | 74/83 | 70/86 | 73/85 | 76/84 | — |
| TaGRF12 | 68/76 | 68/77 | 67/74 | 65/79 | 68/79 | 64/76 | 67/74 |

Sequences used in the comparison (QLQ underlined, WRC bold)

>TraesCS6A01G335900 TaGRF1
(SEQ ID NO: 125)
PFTATQWQELEHQALIYKYMASGVPIPSDLLLPLRRSFDPEPGRCRRTD
GKKWRCSKEAYPDSKYCEKHMHRGKNRSRKPVE
>TraesCS7A01G165600 TaGRF2
(SEQ ID NO: 126)
LFTASQWRELEHQALIYKYMAAGSQVPHELVLPLRHRDDPEPGRCRRTD
GKKWRCSREAYGESKYCDRHMHRGKNRSRKPVE
>TraesCS6A01G269600 TaGRF4
(SEQ ID NO: 127)
PFTAAQYEELEHQALIYKYLVAGVSVPPDLVLPIRRGIDPEPGRCRRTD
GKKWRCAKEAASDSKYCERHMHRGRNRSRKPVE
>TraesCS2A01G435100 TaGRF3
(SEQ ID NO: 128)
TFTAAQYEELEQQALIYKYLVAGVPVPPDLLLPIRRGFDPEPGRCRRTD
GKKWRCSKEAAQDSKYCERHMHRGRNRSRKPVE
>TraesCS7A01G049100 TaGRF5
(SEQ ID NO: 129)
VFTPAQWAELEQQALIYKYLMAGVPVPPDLLLPIRPHPDPEPWRCRRTD
GKKWRCSKEAHPDSKYCERHMHRGRNRSRKPVE
>TraesCS4A01G291500 TaGRF9
(SEQ ID NO: 130)
PFTPSQWMELEHQALIYKYLNAKAPIPSGLLISISKSFDPEPGRCRRTD
GKKWRCSKEAMAEHKYCERHINRNRHRSRKPVE
>TraesCS4A01G255000 TaGRF6
(SEQ ID NO: 131)
PFTPSQWMELEHQALIYKYLAANIAVPHSLLVPIRRSVDLEPGRCRRTD
GKKWRCSRDAVADQKYCERHMNRGRHRSRKHVE
>TraesCS6A01G257600 TaGRF12
(SEQ ID NO: 132)
ALTFMQQQELEHQVLIYRYFAAGAPVPVHLVLPIWKSVEPEPGRCRRTD
GKKWRCSRDVVQGHKYCERHVHRGRGRSRKPVE

Table 9. Regeneration frequencies in plants transformed with the wheat GRF4-GIF1 chimera or the empty vector. A) Tetraploid and hexaploid wheat commercial cultivars. B) Triticale breeding line and C) Rice cultivar Kitaake. In all experiments we used the pLC41 vector (optimized for wheat), except for the second rice experiment where we used the pCAMBIA1300 vector which is more frequently used in rice. EHA105 and AGL1 are two different Agrobacterium strains used for infiltration (we observed no differences between the two strains). In the number of inoculated embryos (n) the first number indicates those inoculated with the empty vector and the second number the embryos inoculated with the GRF4-GIF1 chimera.

9A. Wheat

| Desert King (4×) | Exp1 (EHA105) | Exp2 (EHA105) | | |
|---|---|---|---|---|
| No. embryos inoc. | n = 20/20 | n = 50/50 | Average | SE |
| pLC41 | 0.05 | 0 | 0.025 | 0.025 |
| Ubi::GRF4-GIF1 | 0.80 | 0.46 | 0.630 | 0.170 |
| Fielder (6×) | Exp1 (EHA105) | Exp2 (EHA105) | | |
| No. embryos inoc. | n = 49/67 | n = 10/10 | Average | SE |
| pLC41 | 0.05 | 0 | 0.025 | 0.025 |
| Ubi::GRF4-GIF1 | 0.58 | 0.5 | 0.540 | 0.040 |

| Cadenza (6×) | Exp1 (AGL1) | Exp2 (AGL1) | Exp3 (EHA105) | | |
|---|---|---|---|---|---|
| No. embryos inoc. | n = 19/12 | n = 23/24 | n = 25/25 | Average | SE |
| pLC41 | 0 | 0 | 0 | 0.000 | 0.000 |
| Ubi::GRF4-GIF1 | 0.20 | 0.17 | 0.20 | 0.190 | 0.010 |

| Hahn (6×) | Exp1 (EHA105) | Exp2 (EHA105) | Exp3 (EHA105) | Exp4 (AGL1) | | |
|---|---|---|---|---|---|---|
| No. embryos inoc. | n = 31/37 | n = 48/50 | n = 69/51 | n = 25/25 | Average | SE |
| pLC41 | 0 | 0 | 0 | 0 | 0.000 | 0.000 |
| Ubi::GRF4-GIF1 | 0.03 | 0.04 | 0 | 0.28 | 0.088 | 0.087 |

9B. Triticale

| Triticale UC3190 (6x) | Exp 9 (EHA105) | Exp 11 (EHA105) | Exp 15 (EHA105) | | |
|---|---|---|---|---|---|
| No. embryos inoc. | n = 45/45 | n = 21/22 | n = 42/43 | Average | SE |
| pLC41 | 0 | 0 | 0 | 0.000 | 0.000 |
| Ubi::GRF4-GIF1 | 0.05 | 0.13 | 0.14 | 0.107 | 0.028 |

9C. Rice. Regeneration frequencies in rice (Oryza sativa) cultivar
Kitaake. Both experiments were performed with Agrobacterium strain
EHA105. In the first experiment, we used the wheat-optimized vector
pLC41 and in experiments 2-4 the rice-optimized vector pCAMBIA1300
vector. The first number in the inoculated calli represents the
calli inoculated with the empty vector and the second number the
calli inoculated with the wheat GRF4-GIF1 chimera. Experiments
2-4 are three experiments performed with the same seed stock (100
embryos each). Experiment 2 included the Ubi::GRF4-GIF1 chimera
and a sgRNA targeted to gene OsKitaake06g041700 encoding a Tyrosine
Protein Sulfotransferase (TPST), and experiments 3 and 4 included
pCAMBIA1300-gus without the chimera.

| Rice Kitaake | Exp 1 | Exp 2-4 | | |
|---|---|---|---|---|
| No. calli inoc. | n = 85/85 | n = 200/100 | Average | SE |
| No GRF4-GIF1 | 0.118 | 0.235 | 0.176 | 0.059 |
| Ubi::GRF4-GIF1 | 0.353 | 0.460 | 0.406 | 0.054 |

TABLE 10
Regeneration frequencies in Citrus. Experiments 1 to 3 used a GRF4-GIF1
chimera based on Citrus sequences whereas experiments 4 to 6 used a GRF4-GIF1 chimera based
on Vitis sequences. The last three experiments included a second Vitis construct with mutations in
the miR396 binding site (rGRF4-GIF1) that precludes its cleavage. The first number in the
inoculated calli represents the calli inoculated with the empty vector and the other numbers the
calli inoculated with the different GRF4-GIF1 chimeras.

| Carrizo | Exp1 | Exp2 | Exp3 | Exp4 | Exp5 | Exp6 | | |
|---|---|---|---|---|---|---|---|---|
| No. epicotyls. | n = 45/45 | n = 38/41 | n = 56/69 n = | = 65/59/66 | n = 32/31/31 | n = 40/40 | Average | SE |
| Empty vector | 0.04 | 0.00 | 0.09 | 0.02 | 0.09 | 0.02 | 0.05 | 0.02 |
| Citrus GRF4-GIF1 | 0.15 | 0.39 | 0.10 | — | — | — | 0.21 | 0.09 |
| Vitis GRF4-GIF1 | — | — | — | 0.07 | 0.25 | — | 0.16 | 0.09 |
| Vitis rGRF4-GIFl | — | — | — | 0.20 | 0.61 | 0.30 | 0.37 | 0.12 |

TABLE 11
Regeneration frequencies in Capsicum annuum
Cultivar R&C cayenne (experiments No. 201027/201028) in
40 cotyledon pieces transformed with the GRF4.1-GIF1.1
chimera (SEQ ID NO: 140) and 40 with the empty vector
pEarleyGate100. In the number of inoculated explants,
(n) the first number indicates those inoculated with the
empty vector and the second number the embryos inoculated
with the GRF4.1-GIF1.1 chimera.

| R&C cayenne | Plate 1 | Plate 2 | | |
|---|---|---|---|---|
| No. explants. | n = 20/22 | n = 20/20 | Average | SE |
| Empty vector | 0.00 | 0.10 | 0.05 | 0.05 |
| Capsicum GRF4.1-GIF1.1 | 0.23 | 0.25 | 0.24 | 0.01 |

### LIST OF SEQUENCE IDENTIFIERS

SEQ ID NO:

1: Full vector pLC41 having TaGRF4 nucleotide sequence

2: Full vector pLC41 having TaGIF1 nucleotide sequence

3: TaGRF4-GIF1 chimera nucleotide sequence

4: TaGRF4-GIF1 amino acid sequence

5: Full construct pLC41 wheat GRF4-GIF1 chimera

6: Vitis vinifera GRF4-GIF1 chimera nucleotide sequence

7: Vitis vinifera GRF4-GIF1 chimera protein sequence

8: Full construct pGWB14 having Vitis vinifera GRF4-GIF1 chimera

9: TaGRF1 amino acid sequence

10: TaGRF2 amino acid sequence

11: TaGRF3 amino acid sequence

12: TaGRF4 amino acid sequence

13: TaGRF5 amino acid sequence

14: OsGRF1 amino acid sequence

15: OsGRF2 amino acid sequence

16: OsGRF3 amino acid sequence

17: OsGRF4 amino acid sequence

18: OsGRF5 amino acid sequence

19: AtGRF3 amino acid sequence

20: AtGRF4 amino acid sequence

21: AtGRF5 amino acid sequence

22: AtGRF6 amino acid sequence

23: Vitis vinifera GRF4 (SVIVT01024326001) used in the chimera

24: AtGIF1 amino acid sequence

25: AtGIF2 amino acid sequence

26: AtGIF3 amino acid sequence

27: TaGIF1 amino acid sequence

28: TaGIF2 amino acid sequence

29: TaGIF3 amino acid sequence

30: Vitis vinifera GIF1 (GSVIVT01036262001)

31: Dexamethasone inducible chimera of Vitis vinifera GRF4-GR-GIF1 protein

32: Dexamethasone inducible TaGRF4-GR-GIF1

33: Full vector of pLC41 with wheat GRF4-GR-GIF1 chimera

34: wheat rGRF4-GIF1 with four mutation in miR396 target site

35: Full construct JD635 with wheat GRF4-GIF1 chimera

36: Full construct pLC41 with separate Ubi::GRF4-Ubi::GIF1

37: TaGRF5

38: TaGRF1

39: TaGRF9

40: TaGRF1-GIF1

41: TaGRF5-GIF1

42: TaGRF9-GIF1

43: TaGIF2

44: TaGIF3

45: TaGRF4-GIF2

46: TaGRF4-GIF3

47: Full vector pLC41 marker free with wheat GRF4-GIF1 chimera

48: Citrus GRF4-GIF1

49: Full pGWB14 construct with Citrus GRF4-GIF1

50: Vitis rGRF4-GIF1 miR396 resistant chimera

51 TaGRF4 nucleotide sequence—From SEQ ID NO: 1 vector

52 TaGIF1 nucleotide sequence—From SEQ ID NO: 2 vector

53 wild-type miR396 target site

54: miR396 target site with silent mutations

55: Mature miR396 sequence

56: Protein sequence is FIG. 8

57: LB of pLC41

58: RB of pLC41

59: Zea maize UBIQUITIN promoter

60: HA tag

61: nos terminator

62: 35s promoter

63: HPT

64: TaCas9 from SEQ ID NO: 35

65: TaU6 promoter from SEQ ID NO: 35

66: Guide RNA gene Q from SEQ ID NO: 35

67: Guide RNA scaffold from SEQ ID NO: 35

68: Sequence in FIG. 9 B

69: QLQ domain from SEQ ID NO: 9

70: QLQ domain from SEQ ID NO: 10

71: QLQ domain from SEQ ID NO: 11

72: QLQ domain from SEQ ID NO: 12

73: QLQ domain from SEQ ID NO: 13

74: QLQ domain from SEQ ID NO: 14

75: QLQ domain from SEQ ID NO: 15

76: QLQ domain from SEQ ID NO: 16

77: QLQ domain from SEQ ID NO: 17

78: QLQ domain from SEQ ID NO: 18

79: QLQ domain from SEQ ID NO: 19

80: QLQ domain from SEQ ID NO: 20

81: QLQ domain from SEQ ID NO: 21

82: QLQ domain from SEQ ID NO: 22

83: QLQ domain from SEQ ID NO: 23

84: WRC domain from SEQ ID NO: 9

85: WRC domain from SEQ ID NO: 10

86: WRC domain from SEQ ID NO: 11

87: WRC domain from SEQ ID NO: 12

88: WRC domain from SEQ ID NO: 13

89: WRC domain from SEQ ID NO: 15

90: WRC domain from SEQ ID NO: 16

91: WRC domain from SEQ ID NO: 19

92: WRC domain from SEQ ID NO: 20

93: WRC domain from SEQ ID NO: 21

94: WRC domain from SEQ ID NO: 22

95: WRC domain from SEQ ID NO: 23

96: A concatenated QLQ-WRC domain for wheat TaGRF4 (for which the complete protein sequence is SEQ ID NO:12):

97: SNH domain from SEQ ID NO: 24

98: SNH domain from SEQ ID NO: 25

99: SNH domain from SEQ ID NO: 26

100: SNH domain from SEQ ID NO: 27

101: SNH domain from SEQ ID NO: 28

102: SNH domain from SEQ ID NO: 29

103: SNH domain from SEQ ID NO: 30

104: Primer Fw-GRF4b

105: Primer Rev-GIF1c

106-118: Table 6 primers

119: Primer Fw_HindIII

120: Primer Rev-HindIII

121: Primer Fw-GRF

122: Primer rGRF-Rev

123: Primer rGRF-Fw

124: Primer Rev-GIF

125: QLQ-WRC from TraesCS6A01G335900 TaGRF1

126: QLQ-WRC from TraesCS7A01G165600 TaGRF2

127: QLQ-WRC from TraesCS6A01G269600 TaGRF4

128: QLQ-WRC from TraesCS2A01G435100 TaGRF3

129: QLQ-WRC from TraesCS7A01G049100 TaGRF5

130: QLQ-WRC from TraesCS4A01G291500 TaGRF9

131: QLQ-WRC from TraesCS4A01G255000 TaGRF6

132: QLQ-WRC from TraesCS6A01G257600 TaGRF12

133: Os11g40100 (GIF2)

134: Os12g31350 (GIF3)

135: Os03g52320 (GIF1)

136: OsGIF3 SNH Domain

137: OsGIF1 SNH Domain

138: Capsicum GRF4 (LOC107869915)

139 Capsicum GIF1 (LOC107870303)

140 Capsicum GRF4-GIF1 chimera.

### REFERENCES

All references cited in this disclosure are incorporated by reference in their entirety.
Beltramino, M., Ercoli, M. F., et al. (2018). “Robust increase of leaf size by Arabidopsis thaliana GRF3-like transcription factors under different growth conditions”. Scientific Reports 8:13447.
Che, R., Tong, H., et al. (2015). “Control of grain size and rice yield by GL2-mediated brassinosteroid responses.” Nat Plants 2:15195.
Debernardi, J. M., Mecchia, M. A., et al. (2014). “Post-transcriptional control of GRF transcription factors by microRNA miR396 and GIF co-activator affects leaf size and longevity”. Plant J. 74:413-426.
Debernardi, J. M., Rodriguez, R. E., et al. (2012). “Functional specialization of the plant miR396 regulatory network through distinct microRNA-target interactions”. PLoS Genet 8: e1002419.
Duan, P., Ni, S., et al. (2015). “Regulation of OsGRF4 by OsmiR396 controls grain size and yield in rice”. Nat Plants 2:15203.
He, Z., Zeng, J. et al., (2017). “OsGIF1 positively regulates the sizes of stems, leaves, and grains in rice”. Front Plant Sci 8:1730.
Horiguchi, G., Kim, G. T., et al. (2005). “The transcription factor AtGRF5 and the transcription coactivator AN3 regulate cell proliferation in leaf primordia of Arabidopsis thaliana”. Plant J 43: 68-78.
Hu, J., Wang, Y., et al. (2015). “A rare allele of GS2 enhances grain size and grain yield in rice”. Mol Plant 8:1455-1465.
Kim, H., Kim, S. T., Ryu, J., Kang, B. C., Kim, J. S., Kim, S. G. (2017). “CRISPR/Cpf1-mediated DNA-free plant genome editing”. Nat Commun 8:14406.
Kim, J. H. and B. H. Lee (2006). “GROWTH-REGULATING FACTOR4 of Arabidopsis thaliana is required for development of leaves, cotyledons, and shoot apical meristem.” Journal of Plant Biology 49: 463-468.
Kim, J. H., Choi, D., et al. (2003). “The AtGRF family of putative transcription factors is involved in leaf and cotyledon growth in Arabidopsis”. Plant J 36: 94-104.
Kim, J. H. and Kende, H. (2004). “A transcriptional coactivator, AtGIF1, is involved in regulating leaf growth and morphology in Arabidopsis”. Proc Natl Acad Sci USA 101: 13374-13379.
Lee, B. H., J. H. Ko, et al. (2009). “The Arabidopsis GRF-INTERACTING FACTOR gene family performs an overlapping function in determining organ size as well as multiple developmental properties.” Plant Physiol 151:655-668.
Li, S., Gao, F., et al. (2016). “The OsmiR396c-OsGRF4-OsGIF1 regulatory module determines grain size and yield in rice”. Plant Biotechnol J 14:2134-2146.
Li, S., Tian, Y., et al. (2018). “Modulating plant growth-metabolism coordination for sustainable agriculture”. Nature 560:595-600.
Liang, Z., Chen, K., Li, T., Zhang, Y., Wang, Y., Zhao, Q., Liu, J., Zhang, H., Liu, C., Ran, Y., Gao, C. (2017). “Efficient DNA-free genome editing of bread wheat using CRISPR/Cas9 ribonucleoprotein complexes”. Nat Commun 8:14261.
Liu, D., Song, Y., et al. (2009). “Ectopic expression of miR396 suppresses GRF target gene expression and alters leaf growth in Arabidopsis”. Physiol Plant 136: 223-236.
Malnoy, M., Viola, R., et al. (2016). “DNA-free genetically edited grapevine and apple protoplast using CRISPR/Cas9 ribonucleoproteins”. Front Plant Sci 7:1904
Omidbakhshfard, M. A., Proost, S., et al. (2015). “Growth-Regulating Factors (GRF s): A Small Transcription Factor Family with Important Functions in Plant Biology”. Mol Plant 8: 998-1010.
Rodriguez, R. E., Mecchia, M. A., et al. (2010). “Control of cell proliferation in Arabidopsis thaliana by microRNA miR396”. Development 137: 103-112.
Shimano, S., Hibara, K. I., et al. (2018). “Conserved functional control, but distinct regulation, of cell proliferation in rice and Arabidopsis leaves revealed by comparative analysis of GRF-INTERACTING FACTOR 1 orthologs”. Development 145: dev159624.
Subburaj, S., Chung, S. J., et al. (2016). “Site-directed mutagenesis in Petunia x hybrid protoplast system using direct delivery of purified recombinant Cas9 ribonucleoproteins”. Plant Cell Rep 35:1535-1544.
Sun, P., Zhang, W., et al. (2016). “OsGRF4 controls grain shape, panicle length and seed shattering in rice”. J Integr Plant Biol 58:836-847.
Svitashev, S., Schwartz, C., Lenderts, B., Young, J. K., Mark Cigan, A. (2016). “Genome editing in maize directed by CRISPR-Cas9 ribonucleoprotein complexes”. Nat Commun 7:13274.
van der Knaap, E., Kim, J. H., et al. (2000). “A novel gibberellin-induced gene from rice and its potential regulatory role in stem growth”. Plant Physiol 122: 695-704.
Vercruyssen, L., Verkest, A., et al. (2014). “ANGUSTIFOLIA3 binds to SWI/SNF chromatin remodeling complexes to regulate transcription during Arabidopsis leaf development”. Plant Cell 26: 210-229.
Wang, L., Gu, X., et al. (2011). “miR396-targeted AtGRF transcription factors are required for coordination of cell division and differentiation during leaf development in Arabidopsis”. J Exp Bot 62: 761-773.
Wolter, F., Puchta, H. (2017). “Knocking out consumer concerns and regulator's rules: efficient use of CRISPR/Cas ribonucleoprotein complexes for genome editing in cereals”. Genome Biol 18:43
Woo, J. W., Kim, J., et al. (2015). “DNA-free genome editing in plants with preassembled CRISPR-Cas9 ribonucleoproteins”. Nat Biotechnol 33:1162-1164
Zhang, D., Sun, W., et al (2018). “GRF-INTERACTING FACTOR 1 regulates shoot architecture and meristem determinacy in maize”. Plant Cell. 30:360-374.

1: A method of increasing regeneration efficiency of one or more transgenic plant cells, the method comprising,
a) introducing into said one or more plant cells a nucleic acid molecule encoding or a polypeptide comprising,
i) a Growth Regulating Factor (GRF) polypeptide and/or a GRF-interacting Factor (GIF) polypeptide; or
ii) a GRF-GIF polypeptide chimera; and
b) said one or more plant cells having increased regeneration efficiency compared to a plant not comprising said introduced nucleic acid molecule encoding or a polypeptide comprising a GRF polypeptide and/or a, GIF polypeptide or a GRF-GIF polypeptide chimera.

2: The method of claim 1, further comprising producing a plant from said one or more plant cells.

3: The method of claim 1, wherein said GRF polypeptide is selected from a wheat GRF1, GRF2, GRF3, GRF4, GRF5, GRF6 or GRF 9 polypeptide or a homolog from other plant species or a combination thereof.

4: The method of claim 1, wherein said GIF polypeptide is selected from a wheat GIF1, GIF2, GIF3 polypeptide or a homolog from other plant species thereof or a combination thereof.

5: The method of claim 1, wherein said GIF polypeptide is selected from SEQ ID NO: 24-30, 43-44, 52, 133-135, 139 or a polypeptide sequence having at least 70% identity in the SNH domain thereto, or a combination thereof.

6: The method of claim 1, wherein said GRF polypeptide is selected from SEQ ID NO: 9-23, 37-39, 51, 125-132 or 138 or a polypeptide sequence having at least 70% identity in the QLQ and WRC domains thereto or a combination thereof.

7: The method of claim 1, wherein said GRF polypeptide comprises a QLQ selected from SEQ ID NO: 69-83 and WRC domain that selected from SEQ ID NO: 84-95 or a sequence having at least 70% identity thereto.

8: The method of claim 1, wherein said GIF polypeptide comprises a SNH domain selected from SEQ ID NO: 97-103, 136, 137 or a sequence having at least 70% identity thereto.

9: The method of claim 1, wherein said GRF polypeptide comprises one or more mutations in the miR396 target site and lowering repression of said GRF polypeptide by miR396 in said plant.

10: The method of claim 9, wherein said mutations comprise silent mutations of said miR396 target site.

11: The method of claim 9, wherein said miRNA target site comprises SEQ ID NO: 53

12: The method of claim 1, further comprising an inducible nucleic acid molecule operably linked to said GRF, GIF or GRF-GIF chimera.

13: The method of claim 1, wherein said one more plant cells are regenerated on media comprising cytokinin that is at a concentration not sufficient to regenerate a plant cell that has not had said GRF, GIF or GRF-GIF chimera introduced.

14: The method of claim 13, wherein said one or more transformed plant cells comprising said heterologous nucleic acid molecule or polypeptide is selected by its ability to grow on said media.

15: The method of claim 1, wherein said plant has at least 10% increased regeneration efficiency compared to a plant cell not comprising said introduced GRF, GIF or GRF-GIF chimeras.

16: The method of claim 1, wherein said plant is selected from Triticum, Oryza, Vitis, Citrus, avocado, walnut, pistachio, peach, apple, cherry, strawberry, blueberry, raspberry, bean, broccoli, cauliflower, cowpea, leek, melon, onion, pepper, spinach, squash or watermelon.

17: The method of claim 1, wherein said plant comprises an elite plant.

18: The method of claim 1, wherein said one or more plant cells comprises one or more leaf explant cells, or cell from any other tissue.

19: The method of claim 1, wherein said nucleic acid molecule or polypeptide of said GRF, GIF or GRF and GIF chimera is combined with CRISPR-CAS9 or any other gene editing system.

20: The method of claim 19, wherein said construct combining GRF, GIF or GRF and GIF chimera with gene editing is removed from said plant by segregation in the progeny of the edited plant.

21: The method of claim 1, wherein said nucleic acid molecule encoding or polypeptide comprising a GRF, GIF or GRF-GIF chimera polypeptide are introduced into said plant prior to introducing said heterologous nucleic acid molecule or polypeptide.

22: A method of increasing regeneration efficiency of a plant, wherein said plant contains miR396, the method comprising
a) introducing into said plant a nucleic acid molecule encoding or a polypeptide comprising,
i) a Growth Regulating Factor (GRF) polypeptide and/or a GRF-interacting Factor (GIF) polypeptide; or
ii) a GRF-GIF polypeptide chimera; and
b) said GRF polypeptide comprising a miR396 target site having one or more mutations and lowering repression of said GRF polypeptide by said miRNA in said plant.

23: The method of claim 22, wherein said plant is a transgenic plant.

24: A plant produced by the method of claim 2.

25: The method of claim 1 where GRF, GIF or GRF-GIF chimers are used to accelerate the time required to regenerate plants.


SEQUENCE LISTING (140 sequences)

SEQ ID NO: 1 - DNA, 16633 bp, Artificial Sequence
  Synthetic construct
aagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgagatgtaccgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcactagtaacggccgccagtgtgctggaattcgccctttaaataagcgatcgccccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgtcgagggggatccaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatttaaattgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgggcgcggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagc

SEQ ID NO: 2 - DNA, 16087 bp, Artificial Sequence
  Synthetic construct
aagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagccgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcactagtaacggccgccagtgtgctggaattcgccctttaaataagcgatcgccccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgtcgagggggatccaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatttaaattgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgggcgcggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagc

SEQ ID NO: 3 - DNA, 1911 bp, Artificial Sequence
  Synthetic construct
atggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgaaatgtacgcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctag

SEQ ID NO: 4 - protein, 636 aa, Artificial Sequence
  Synthetic construct
MAMPYASLSPAGDRRSSPAATATASLLPFCRSSPFSAGGNGGMGEEARMDGRWMARPVPFTAAQYEELEHQALIYKYLVAGVSVPPDLVLPIRRGIESLAARFYHNPLAIGYGSYLGKKVDPEPGRCRRTDGKKWRCAKEAASDSKYCERHMHRGRNRSRKPVETQLVSHSQPPAASVVPPLATGFHNHSLYPAIGGTNGGGGGGNNGMSMPGTFSSALGPPQQHMGNNAASPYAALGGAGTCKDFRYTAYGIRSLADEQSQLMTEAMNTSVENPWRLPPSSQTTTFPLSSYSPQLGATSDLGQNNSSNNNSGVKAEGQQQQQPLSFPGCGDFGSGDSAKQENQTLRPFFDEWPKTRDSWSDLTDDNSNVASFSATQLSISIPMTSSDFSAASSQSPNGMLFAGEMYAAAAMQQQHLMQMNQSMMGGYASSTTATTDLIQQYLDENKQLILAILDNQNNGKVEECARNQAKLQQNLMYLAAIADSQPPQTASLSQYPSNLMMQSGPRYMQQQSAQMMSPQSLMAARSSMMYAQQAMSPLQQQQQQQQHQAAAHGQLGMSSGATTGFNLLHGEASMGGGGGATGNSMMNASVFSDYGRGGSGAKEGSTSLSADARGANSGAHSGDGEYLKGTEEEGS

SEQ ID NO: 5 - DNA, 17323 bp, Artificial Sequence
  Synthetic construct
aagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgaaatgtacgcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctagcgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcactagtaacggccgccagtgtgctggaattcgccctttaaataagcgatcgccccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgtcgagggggatccaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatttaaattgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgggcgcggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagc

SEQ ID NO: 6 - DNA, 1698 bp, Artificial Sequence
  Synthetic construct
atgaagcaaagctttgtgggggtttcttcacaaacagaggctgagacaagagccagagagagtgacacacagacacacccacaaaggaacctcagtcactgtccccaaaaacatcctcaaaacttgcaaagaacccaaaatggtgggtctgttgggggggcatcaatggcaacagggatgatggtgaggtcatcaccattcacagtgtctcagtggcaggagctggagcatcaagctttgatctttaagtatctaatggcaggactacccgtcccacctgatcttgtgcttcctattcagaagagctttgaatctatgtcttctaggttcttccaccatcctaccatgggttactgttccttctatgggaagaaggtggacccggagccaggtagatgccggaggactgatggcaagaaatggaggtgctccaaagacgcctacccggactccaagtactgtgagcgccacatgcaccgtggccgcaaccgttcaagaaagcctgtggaatcacaaactactacacagtcttcttcgactgtgacttcactaactgtcactggaggcagtggcagtggagccgggaccttccagaaccttcccctgcaaaacttcaataacccccaaggcggcccggggtccggaaccaaccaatcccaattccatatggagcccattccctatggcattcctaataaagattacaggtatcttcatggacttaaacccgaggtaggcgaacatagtttcttctctgaagcttcaggaagcaacaggggtcttcagatggactcgcctttggacagcacatggcctctgatgccatcacgggtctcatcattccccccatcaaaatcaagtgaaaactccatcttgcagagtgactacccccagcattcgttcttcagcagcgaatttgcgtcgggagaggttgtgaaacaggagggtcagtctctccgtcctttctttgacgagtggcctaaaaccagagactcctggtccggccttgaggaagagaggtccaaccagacctcattctccacaacccagctctcaatttcgatcccaatggcctcctctgacttctcaacaaccagctctcggtctccccacgataacgcggccgctgccatgcagcccatgatggcagcctattaccccagcaacgtcaccactgatcacattcagcagtatcttgatgaaaacaagtcattgattctgaagattgttgagagccagaattcaggaaaattgactgaatgtgcagagaaccaggcaagactacagagaaacctcatgtacctggctgcaatcgctgattctcaaccccaaccacccaccatgcatgctcagttccctcctagtggcattgttcagccaggagctcactacatgcagcaccaacaagctcaacaaatgacaccacagtcgctcctggctgcacgctcctccatgctgtactcccaacaaccattttcggccctgcaacaacaacaagccatccatagccagcttggcatgggctctggtggaagtgcaggacttcacatgctgcaaagcgaggggagtaatccaggaggcaatggaacactggggactgatatgggaaatgcagaagggcgaggagggaactcaggaggtcagggtggggatggaggtgagactctttacttgaaagctgctgaagatgggaattga

SEQ ID NO: 7 - protein, 565 aa, Artificial Sequence
  Synthetic construct
MKQSFVGVSSQTEAETRARESDTQTHPQRNLSHCPQKHPQNLQRTQNGGSVGGASMATGMMVRSSPFTVSQWQELEHQALIFKYLMAGLPVPPDLVLPIQKSFESMSSRFFHHPTMGYCSFYGKKVDPEPGRCRRTDGKKWRCSKDAYPDSKYCERHMHRGRNRSRKPVESQTTTQSSSTVTSLTVTGGSGSGAGTFQNLPLQNFNNPQGGPGSGTNQSQFHMEPIPYGIPNKDYRYLHGLKPEVGEHSFFSEASGSNRGLQMDSPLDSTWPLMPSRVSSFPPSKSSENSILQSDYPQHSFFSSEFASGEVVKQEGQSLRPFFDEWPKTRDSWSGLEEERSNQTSFSTTQLSISIPMASSDFSTTSSRSPHDNAAAAMQPMMAAYYPSNVTTDHIQQYLDENKSLILKIVESQNSGKLTECAENQARLQRNLMYLAAIADSQPQPPTMHAQFPPSGIVQPGAHYMQHQQAQQMTPQSLLAARSSMLYSQQPFSALQQQQAIHSQLGMGSGGSAGLHMLQSEGSNPGGNGTLGTDMGNAEGRGGNSGGQGGDGGETLYLKAAEDGN

SEQ ID NO: 8 - DNA, 17390 bp, Artificial Sequence
  Synthetic construct
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagctgccaccatgaagcaaagctttgtgggggtttcttcacaaacagaggctgagacaagagccagagagagtgacacacagacacacccacaaaggaacctcagtcactgtccccaaaaacatcctcaaaacttgcaaagaacccaaaatggtgggtctgttgggggggcatcaatggcaacagggatgatggtgaggtcatcaccattcacagtgtctcagtggcaggagctggagcatcaagctttgatctttaagtatctaatggcaggactacccgtcccacctgatcttgtgcttcctattcagaagagctttgaatctatgtcttctaggttcttccaccatcctaccatgggttactgttccttctatgggaagaaggtggacccggagccaggtagatgccggaggactgatggcaagaaatggaggtgctccaaagacgcctacccggactccaagtactgtgagcgccacatgcaccgtggccgcaaccgttcaagaaagcctgtggaatcacaaactactacacagtcttcttcgactgtgacttcactaactgtcactggaggcagtggcagtggagccgggaccttccagaaccttcccctgcaaaacttcaataacccccaaggcggcccggggtccggaaccaaccaatcccaattccatatggagcccattccctatggcattcctaataaagattacaggtatcttcatggacttaaacccgaggtaggcgaacatagtttcttctctgaagcttcaggaagcaacaggggtcttcagatggactcgcctttggacagcacatggcctctgatgccatcacgggtctcatcattccccccatcaaaatcaagtgaaaactccatcttgcagagtgactacccccagcattcgttcttcagcagcgaatttgcgtcgggagaggttgtgaaacaggagggtcagtctctccgtcctttctttgacgagtggcctaaaaccagagactcctggtccggccttgaggaagagaggtccaaccagacctcattctccacaacccagctctcaatttcgatcccaatggcctcctctgacttctcaacaaccagctctcggtctccccacgataacgcggccgctgccatgcagcccatgatggcagcctattaccccagcaacgtcaccactgatcacattcagcagtatcttgatgaaaacaagtcattgattctgaagattgttgagagccagaattcaggaaaattgactgaatgtgcagagaaccaggcaagactacagagaaacctcatgtacctggctgcaatcgctgattctcaaccccaaccacccaccatgcatgctcagttccctcctagtggcattgttcagccaggagctcactacatgcagcaccaacaagctcaacaaatgacaccacagtcgctcctggctgcacgctcctccatgctgtactcccaacaaccattttcggccctgcaacaacaacaagccatccatagccagcttggcatgggctctggtggaagtgcaggacttcacatgctgcaaagcgaggggagtaatccaggaggcaatggaacactggggactgatatgggaaatgcagaagggcgaggagggaactcaggaggtcagggtggggatggaggtgagactctttacttgaaagctgctgaagatgggaattgacgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttcatcaacgcaagacatgcgcacgaccgtctgacaggagaggaatttccgacgagcacagaaaggacttgctcttggacgtaggcctatttctcaggcacatgtatcaagtgttcggacgtgggttttcgatggtgtatcagccgccgccaactgggagatgaggaggctttcttggggggcagtcagcagttcatttcacaagacagaggaacttgtaaggagatgcactgatttatcttggcgcaaaccagcaggacgaattagtgggaatagcccgcgaatatctaagttatgcctgtcggcatgagcagaaacttccaattcgaaacagtttggagaggttgtttttgggcataccttttgttagtcagcctctcgattgctcatcgtcattacacagtaccgaagtttgatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtatttcgattttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcggggtcgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacggatctctaggacgcgtcctagaagctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 9 - protein, 409 aa, Triticum aestivum
MMMMGGRAGAGGVGAGGGRCPFTATQWQELEHQALIYKYMASGVPIPSDLLLPLRRSFLLDSALATSPSLAFPPQAALGWGCFGMGFGRKAEDPEPGRCRRTDGKKWRCSKEAYPDSKYCEKHMHRGKNRSRKPVEMSLATPPPPPSSSASSSSSNVHSAVNVATTTTSPAPSYHRHAAATHDTTPYHALYGGPYSSAGRQQHASAYHHAAQVSPFHLHLDTTHPHPPPSYYSTMDHSKDSYAYGHSVKEVHGGGEHAFFSSDVSTDRDHHHHQHQHHASAGGNGQWQFKQLGGMEPKQHNPTSLFPGYGNNAAYAIDLSSKEEDEEKERRQQQQHCFLLGADLRLDKPSSGHGDSADQKPLRPFFDEWPHEKTGSKGSWMGLEGETQLSISIANELPITTTSRYHHGE

SEQ ID NO: 10 - protein, 311 aa, Triticum aestivum
MMLGGHGGGGGRCLFTASQWRELEHQALIYKYMAAGSQVPHELVLPLRHRDAAAFAGIDTAPSVACYPPPQPSLGWGLYGAGAQYARKPEDPEPGRCRRTDGKKWRCSREAYGESKYCDRHMHRGKNRSRKPVEPMSSSSVSSPAASYRQTTLSMSPPTPADTPSYGHGHLRAAASQSQINPLQLHLDTPSPPPSYHRYAPAQQYGGSFFPSRQQVQEEAEAEARRRQHFLALGADLSLDKPDATTAASSTTEEKPLRRFFDEWPRDGNAVEVRPWNMGHRDETLLSMSIPTTTASHPDLAAYRHHNGTFA

SEQ ID NO: 11 - protein, 384 aa, Triticum aestivum
MAMPFASLSPAADHHRSSPIFPFCRSSPLYSAGEEAAQQQQQQQHAMSGARWAAARPATFTAAQYEELEQQALIYKYLVAGVPVPPDLLLPIRRGFDSLASRFYHHHALGYGSYFGKKLDPEPGRCRRTDGKKWRCSKEAAQDSKYCERHMHRGRNRSRKPVETQLVSHSQQLQQQAPAAAFHGHSPYPAIATGAGAPGSFALGSTAQLHMDNAAAPYATAGAAGNKDFRYSAYGFRTSAMEDHNQFISAAMDTAMDNYSWRLLPAQNSSFSLSSYPMLSTLSDLDQSAICSLAKTEREPLSFFGVGGGFDDDESAVKQENQTLRPFFDEWPKDRDSWPELQDHDSNHNNEAFSATKLSISIPVTSSDFSTTAGSRSPHGIYSR

SEQ ID NO: 12 - protein, 408 aa, Triticum aestivum
MAMPYASLSPAGDRRSSPAATATASLLPFCRSSPFSAGGNGGMGEEARMDGRWMARPVPFTAAQYEELEHQALIYKYLVAGVSVPPDLVLPIRRGIESLAARFYHNPLAIGYGSYLGKKVDPEPGRCRRTDGKKWRCAKEAASDSKYCERHMHRGRNRSRKPVETQLVPHSQPPAASAVPPLATGFHSHSLYPAIGGGTNGGGGGGNNGMSMPGTFSSALGPPQQHMGNNAASPYAALGGAGTCKDFRYTAYGIRSLADEQSQLMTEAMNTSVENPWRLPPSSQTTTFPLSSYSPQLGATSDLGQNNSSNNNSGVKAEGQQQQQPLSFPGCGDFGSGDSAKQENQTLRPFFDEWPKTRDSWSDLTDDNSNVASFSATQLSISIPMTSSDFSAASSQSPNGMLFAGEMY

SEQ ID NO: 13 - protein, 368 aa, Triticum aestivum
MLSSSAAMGMGLGGYGQQQQQQQMQMQMQRGAGPVFTPAQWAELEQQALIYKYLMAGVPVPPDLLLPIRPHPAGAGATFSFANPAASPFYHHHHPSMSYYAYYGKKLDPEPWRCRRTDGKKWRCSKEAHPDSKYCERHMHRGRNRSRKPVESKSASPAHQSQQPPLSAVTSATRDAEPLPSLPAGAKTHGLSLGGAGSSQMHVDASSYGNKYSLGAKSDVGELSFFSGASGNNNRGFTIDSPTDSSWHSMGSSLPPYQLSKPRDSGLMQGGFSYSHFEPSQELGQVTIASLSHSQEQDRRSFGGGGGGGGGGAGLMGNVKQENQPLRPFFDEWPGRRDSWSEMDDERSNGTSFSTTQLSISIPMPRCD

SEQ ID NO: 14 - protein, 397 aa, Oryza sativa
MMMMSGRPSGGAGGGRYPFTASQWQELEHQALIYKYMASGTPIPSDLILPLRRSFLLDSALATSPSLAFPPQPSLGWGCFGMGFGRKAEDPEPGRCRRTDGKKWRCSKEAYPDSKYCEKHMHRGKNRSRKPVEMSLATPPPPSSSATSAASNTSAGVAPTTTTTSSPAPSYSRPAPHDAAPYQALYGGPYAAATARTPAAAAYHAQVSPFHLQLDTTHPHPPPSYYSMDHKEYAYGHATKEVHGEHAFFSDGTEREHHHAAAGHGQWQFKQLGMEPKQSTTPLFPGAGYGHTAASPYAIDLSKEDDDEKERRQQQQQQQQQHCFLLGADLRLEKPAGHDHAAAAQKPLRHFFDEWPHEKNSKGSWMGLEGETQLSMSIPMAANDLPITTTSRYHNDD

SEQ ID NO: 15 - protein, 301 aa, Oryza sativa
MMAGGGSGRCLFTATQWQELEHQALIYKYMAAGAPVPPDLLLHLRHRAAAAAAADVDTVPSLAFPPHHLGWGCYGAAAAQYGRRVEDPEPGRCRRTDGKKWRCSREAYGESKYCEKHMHRGKNRSRKPVEMPPPAAAAVYRPSALSISPPPHDADAPSYGAGAGAPLQLHLDSFHASTSPPPSYHRYAHTSSAPLFPSSAAGYGGGWSLSKEHCLTLGGAAADLSLDKPADHHHDATSATTEKPLRRFFDEWPRSDDGRTPWDGTQLSISIPTAAAASPDLAIAGAASRYHSNGDHLRTSE

SEQ ID NO: 16 - protein, 387 aa, Oryza sativa
MAMPFASLSPAADHRPSFIFPFCRSSPLSAVGEEAQQHMMGARWAAAVARPPPFTAAQYEELEQQALIYKYLVAGVPVPADLLLPIRRGLDSLASRFYHHPVLGYGSYFGKKLDPEPGRCRRTDGKKWRCSKEAAPDSKYCERHMHRGRNRSRKPVEAQLVAPHSQPPATAPAAAVTSTAFQNHSLYPAIANGGGANGGGGGGGGGGSAPGSFALGSNTQLHMDNAASYSTVAAGAGNKDFRYSAYGVRPLADEHSPLITGAMDTSIDNSWCLLPSQTSTFSVSSYPMLGNLSELDQNTICSLPKVEREPLSFFGSDYVTVDSGKQENQTLRPFFDEWPKARDSWPDLADDNSLATFSATQLSISIPMATSDFSTTSSRSHNGIYSR

SEQ ID NO: 17 - protein, 422 aa, Oryza sativa
MPPCLRRWPTTARPRQPRPPPSSPSAAPPRSPRKQREPAATTHFLGSSGACDNTVRRCVWVGGCRGGGGVAMGEDAPMTARWPPAAAARLPPFTAAQYEELEQQALIYKYLVAGVPVPPDLVLPIRRGLDSLAARFYNHPALGYGPYFGKKLDPEPGRCRRTDGKKWRCSKEAAPDSKYCERHMHRGRNRSRKPVETQLVAQSQPPSSVVGSAAAPLAAASNGSSFQNHSLYPAIAGSNGGGGGRNMPSSFGSALGSQLHMDNAAPYAAVGGGTGKDLRYTAYGTRSLADEQSQLITEAINTSIENPWRLLPSQNSPFPLSSYSQLGALSDLGQNTPSSLSKVQRQPLSFFGNDYAAVDSVKQENQTLRPFFDEWPKGRDSWSDLADENANLSSFSGTQLSISIPMASSDFSAASSRSTNGD

SEQ ID NO: 18 - protein, 356 aa, Oryza sativa
MLSSSPSAAAPGIGGYQPQRGAAVFTAAQWAELEQQALIYKYLVAGVPVPGDLLLPIRPHSSAAATYSFANPAAAPFYHHHHHPSLSYYAYYGKKLDPEPWRCRRTDGKKWRCSKEAHPDSKYCERHMHRGRNRSRKPVESKTAAPAPQSQPQLSNVTTATHDTDAPLPSLTVGAKTHGLSLGGAGSSQFHVDAPSYGSKYSLGAKADVGELSFFSGASGNTRGFTIDSPTDSSWHSLPSSVPPYPMSKPRDSGLLPGAYSYSHLEPSQELGQVTIASLSQEQERRSFGGGAGGMLGNVKHENQPLRPFFDEWPGRRDSWSEMDEERSNQTSFSTTQLSISIPMPRCGSPIGPRLP

SEQ ID NO: 19 - protein, 398 aa, Arabidopsis thaliana
MDLQLKQWRSQQQQQHQTESEEQPSAAKIPKHVFDQIHSHTATSTALPLFTPEPTSSKLSSLSPDSSSRFPKMGSFFSWAQWQELELQALIYRYMLAGAAVPQELLLPIKKSLLHLSPSYFLHHPLQHLPHYQPAWYLGRAAMDPEPGRCRRTDGKKWRCSRDVFAGHKYCERHMHRGRNRSRKPVETPTTVNATATSMASSVAAAATTTTATTTSTFAFGGGGGSEEVVGQGGSFFFSGSSNSSSELLHLSQSCSEMKQESNNMNNKRPYESHIGFSNNRSDGGHILRPFFDDWPRSSLQEADNSSSPMSSATCLSISMPGNSSSDVSLKLSTGNEEGARSNNNGRDQQNMSWWSGGGSNHHHHNMGGPLAEALRSSSSSSPTSVLHQLGVSTQAFH

SEQ ID NO: 20 - protein, 380 aa, Arabidopsis thaliana
MDLQLKQWRSQQQNESEEQGSAATKISNFFFDQIQSQTATSAAAAPLPLFVPEPTSSSSFSCFSPDSSNSSSSSRFLKMGNFFSWAQWQELELQALIYRYMLAGASVPQELLLPIKKSLLHQSPMHFLHHPLQHSFPHHQPSWYWGRGAMDPEPGRCKRTDGKKWRCSRDVVAGHKYCDRHIHRGRNRSRKPVETATTTITTTATTTASSFVLGEELGHGPNNNHFFSSGSSQPLHLSHQQSCSSEMKQESNNNKRPYEANSGFSNGRSDDGHILRHFFDDWPRSSDSTSSPMSSSTCHLSISMPGNNTSSDVSLKLSTGNEEEEENMRNNNNEREQMNWWSNGGNHHNNMGGPLAEALRSASSTSSVLHQMGISTQVFH

SEQ ID NO: 21 - protein, 397 aa, Arabidopsis thaliana
MMSLSGSSGRTIGRPPFTPTQWEELEHQALIYKYMVSGVPVPPELIFSIRRSLDTSLVSRLLPHQSLGWGCYQMGFGRKPDPEPGRCRRTDGKKWRCSREAYPDSKYCEKHMHRGRNRARKSLDQNQTTTTPLTSPSLSFTNNNNPSPTLSSSSSSNSSSTTYSASSSSMDAYSNSNRFGLGGSSSNTRGYFNSHSLDYPYPSTSPKQQQQTLHHASALSLHQNTNSTSQFNVLASATDHKDFRYFQGIGERVGGVGERTFFPEASRSFQDSPYHHHQQPLATVMNDPYHHCSTDHNKIDHHHTYSSSSSSQHLHHDHDHRQQQCFVLGADMFNKPTRSVLANSSRQDQNQEEDEKDSSESSKKSLHHFFGEDWAQNKNSSDSWLDLSSHSRLDTGS

SEQ ID NO: 22 - protein, 244 aa, Arabidopsis thaliana
MATRIPFTESQWEELENQALVFKYLAANMPVPPHLLFLIKRPFLFSSSSSSSSSSSFFSPTLSPHFGWNVYEMGMGRKIDAEPGRCRRTDGKKWRCSKEAYPDSKYCERHMHRGKNRSSSRKPPPTQFTPNLFLDSSSRRRRSGYMDDFFSIEPSGSIKSCSGSAMEDNDDGSCRGINNEEKQPDRHCFILGTDLRTRERPLMLEEKLKQRDHDNEEEQGSKRFYRFLDEWPSSKSSVSTSLFI

SEQ ID NO: 23 - protein, 373 aa, Vitis vinifera
MKQSFVGVSSQTEAETRARESDTQTHPQRNLSHCPQKHPQNLQRTQNGGSVGGASMATGMMVRSSPFTVSQWQELEHQALIFKYLMAGLPVPPDLVLPIQKSFESMSSRFFHHPTMGYCSFYGKKVDPEPGRCRRTDGKKWRCSKDAYPDSKYCERHMHRGRNRSRKPVESQTTTQSSSTVTSLTVTGGSGSGAGTFQNLPLQNFNNPQGGPGSGTNQSQFHMEPIPYGIPNKDYRYLHGLKPEVGEHSFFSEASGSNRGLQMDSPLDSTWPLMPSRVSSFPPSKSSENSILQSDYPQHSFFSSEFASGEVVKQEGQSLRPFFDEWPKTRDSWSGLEEERSNQTSFSTTQLSISIPMASSDFSTTSSRSPHDN

SEQ ID NO: 24 - protein, 210 aa, Arabidopsis thaliana
MQQHLMQMQPMMAGYYPSNVTSDHIQQYLDENKSLILKIVESQNSGKLSECAENQARLQRNLMYLAAIADSQPQPPSVHSQYGSAGGGMIQGEGGSHYLQQQQATQQQQMTQQSLMAARSSMLYAQQQQQQQPYATLQHQQLHHSQLGMSSSSGGGGSSGLHILQGEAGGFHDFGRGKPEMGSGGGGEGRGGSSGDGGETLYLKSSDDGN

SEQ ID NO: 25 - protein, 195 aa, Arabidopsis thaliana
MQQQQSPQMFPMVPSIPPANNITTEQIQKYLDENKKLIMAIMENQNLGKLAECAQYQALLQKNLMYLAAIADAQPPPPTPGPSPSTAVAAQMATPHSGMQPPSYFMQHPQASPAGIFAPRGPLQFGSPLQFQDPQQQQQIHQQAMQGHMGIRPMGMTNNGMQHAMQQPETGLGGNVGLRGGKQDGADGQGKDDGK

SEQ ID NO: 26 - protein, 223 aa, Arabidopsis thaliana
MQQSPQMIPMVLPSFPPTNNITTEQIQKYLDENKKLIMAILENQNLGKLAECAQYQALLQKNLMYLAAIADAQPQPPAATLTSGAMTPQAMAPNPSSMQPPPSYFMQQHQAVGMAQQIPPGIFPPRGPLQFGSPHQFLDPQQQLHQQAMQGHMGIRPMGLNNNNGLQHQMHHHETALAANNAGPNDASGGGKPDGTNMSQSGADGQGGSAARHGGGDAKTEGK

SEQ ID NO: 27 - protein, 226 aa, Triticum aestivum
MQQQHLMQMNQSMMGGYASSTTVTTDLIQQYLDENKQLILAILDNQNNGKVEECARNQAKLQQNLMYLAAIADSQPPQTASLSQYPSNLMMQSGPRYMQQQSAQMMSPQSLMAARSSMMYAQQAMSPLQQQQQQQQQHQAASHGQLGMSSGATTGFNLLHGEASMGGGGGATGNSMMNASVFSDYGRGGSGAKEGSTSLSADARGANSGAHSGDGEYLKGTEEEGS

SEQ ID NO: 28 - protein, 201 aa, Triticum aestivum
MQQAMSLPPGAVGAVSPPAGITTEQIQKYLDENKQLILAILENQNLGKLAECAQYQAQLQKNLLYLAAIADAQPPQNPASRPQMVQQPGSMQGAGHYMSQVPMFPPRTPLTPQQMQEQQHQQLQQQQAQALSFPAQVVMRPGTVNGMQQPMQAAGDLQPAAAPGGSKQDAAVAGASSEPSGTKSHKNAGAEEVGADVAEQS

SEQ ID NO: 29 - protein, 185 aa, Triticum aestivum
MQQAMPMPPAAAAPGMPPSAGLSTEQIQKYLDENKQLILAILENQNLGKLAECAQYQAQLQKNLLYLAAIADTQPQTTVSRPQMAPPSASPGAGHYMSQVPMFPPRTPLTPQQMQEQQLQQQQAQMLPFAGQMVARPGAVNGMPQAPQVEPAYAAGGASSEPSGTESHRSTGADNDGGSGLADQS

SEQ ID NO: 30 - protein, 188 aa, Vitis vinifera
MQPMMAAYYPSNVTTDHIQQYLDENKSLILKIVESQNSGKLTECAENQARLQRNLMYLAAIADSQPQPPTMHAQFPPSGIVQPGAHYMQHQQAQQMTPQSLLAARSSMLYSQQPFSALQQQQAIHSQLGMGSGGSAGLHMLQSEGSNPGGNGTLGTDMGNAEGRGGNSGGQGGDGGETLYLKAAEDGN

SEQ ID NO: 31 - protein, 843 aa, Artificial Sequence
  Synthetic construct
MKQSFVGVSSQTEAETRARESDTQTHPQRNLSHCPQKHPQNLQRTQNGGSVGGASMATGMMVRSSPFTVSQWQELEHQALIFKYLMAGLPVPPDLVLPIQKSFESMSSRFFHHPTMGYCSFYGKKVDPEPGRCRRTDGKKWRCSKDAYPDSKYCERHMHRGRNRSRKPVESQTTTQSSSTVTSLTVTGGSGSGAGTFQNLPLQNFNNPQGGPGSGTNQSQFHMEPIPYGIPNKDYRYLHGLKPEVGEHSFFSEASGSNRGLQMDSPLDSTWPLMPSRVSSFPPSKSSENSILQSDYPQHSFFSSEFASGEVVKQEGQSLRPFFDEWPKTRDSWSGLEEERSNQTSFSTTQLSISIPMASSDFSTTSSRSPHDNAAIQQATAGVSQDTSENPNKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQKAAAMQPMMAAYYPSNVTTDHIQQYLDENKSLILKIVESQNSGKLTECAENQARLQRNLMYLAAIADSQPQPPTMHAQFPPSGIVQPGAHYMQHQQAQQMTPQSLLAARSSMLYSQQPFSALQQQQAIHSQLGMGSGGSAGLHMLQSEGSNPGGNGTLGTDMGNAEGRGGNSGGQGGDGGETLYLKAAEDGN

SEQ ID NO: 32 - protein, 917 aa, Artificial Sequence
  Synthetic construct
MAMPYASLSPAGDRRSSPAATATASLLPFCRSSPFSAGGNGGMGEEARMDGRWMARPVPFTAAQYEELEHQALIYKYLVAGVSVPPDLVLPIRRGIESLAARFYHNPLAIGYGSYLGKKVDPEPGRCRRTDGKKWRCAKEAASDSKYCERHMHRGRNRSRKPVETQLVSHSQPPAASVVPPLATGFHNHSLYPAIGGTNGGGGGGNNGMSMPGTFSSALGPPQQHMGNNAASPYAALGGAGTCKDFRYTAYGIRSLADEQSQLMTEAMNTSVENPWRLPPSSQTTTFPLSSYSPQLGATSDLGQNNSSNNNSGVKAEGQQQQQPLSFPGCGDFGSGDSAKQENQTLRPFFDEWPKTRDSWSDLTDDNSNVASFSATQLSISIPMTSSDFSAASSQSPNGMLFAGEMYAAAAIQQATAGVSQDTSENPNKTIVPAALPQLTPTLVSLLEVIEPEVLYAGYDSSVPDSAWRIMTTLNMLGGRQVIAAVKWAKAIPGLRNLHLDDQMTLLQYSWMFLMAFALGWRSYRQSSGNLLCFAPDLIINEQRMSLPCMYDQCKHMLFVSSELQRLQVSYEEYLCMKTLLLLSSVPKEGLKSQELFDEIRMTYIKELGKAIVKREGNSSQNWQRFYQLTKLLDSMHEVVENLLTYCFQTFLDKTMSIEFPEMLAEIITNQIPKYSNGNIKKLLFHQKAAAAMQQQHLMQMNQSMMGGYASSTTATTDLIQQYLDENKQLILAILDNQNNGKVEECARNQAKLQQNLMYLAAIADSQPPQTASLSQYPSNLMMQSGPRYMQQQSAQMMSPQSLMAARSSMMYAQQAMSPLQQQQQQQQHQAAAHGQLGMSSGATTGFNLLHGEASMGGGGGATGNSMMNASVFSDYGRGGSGAKEGSTSLSADARGANSGAHSGDGEYLKGTEEEGS

SEQ ID NO: 33 - DNA, 18166 bp, Artificial Sequence
  Synthetic construct
aagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgaaatgtacgcggccgctgccattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaagctgccgcggccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctagcgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcactagtaacggccgccagtgtgctggaattcgccctttaaataagcgatcgccccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgtcgagggggatccaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatttaaattgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgggcgcggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagc

SEQ ID NO: 34 - DNA, 1911 bp, Artificial Sequence
  Synthetic construct
atggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttctagaaaaccggtcgaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgaaatgtacgcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctag

SEQ ID NO: 35 - DNA, 21282 bp, Artificial Sequence
  Synthetic construct
tccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatcccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgaacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggacacgctgtcgaacttttcgatcagaaacttctcgacagacgtcgcggtgagttcaggctttttcataagcttctgcaaaagagaaccagacaacagggtaagtgcctagcagtaaacaaacagaactcatcacaagcaaacagcaacatcatattcataccaacaggtcatgtgtgttcatcacatcattagtactaagcatgccatcatccaagtatatcaaagtaagggcaaagagcatgcatgatcatcaggtgcacaaaagaatcatcaaattgtagcagtacatatcttcatctatcatgcatatctatccataacaggacgatgcatgttgaccaggtaaaagctacaggatcctataggaacagcagcgtatatatcttcaccaatcttagcatcttaatcatgtggcacatgcagtttcaatttaagcacatgagctagttgattatgaggtaccagagaatcatcaaactgatgtagcagcatatatcctcatctatcatgcaatctaatctaatctaactaaacaggaaaggtgtgctattcagttaaaagctaccgcatcatatacaaacggcagcataagaaaaagcataatcatcttaatcatgcaacaaacgcagattcataataagcccaagagctagcttgtgatgatcttattctactctgatctacagcaatcagataacgacctaaccttgcacatggcaacaaaacaatcgatcggacgaatcagttgtttgttcctagctagcaccatcgaaccagataatagatgcacgtacagatcccgaaaacgaacccaaaaacagggcagacctagctgaacctaggcagcgacccagcagatcgtgagaacgatctcatctacgaacagcctagaagcaaccccacgattcccggacaaacgacctaaaatccccacaaatcacatgagcatgacaggataaacagcggaaccgatcagatctacacgaaaaccccacctcccagccacccacgatcaggaaacacgcggatctagcatgatttcgtcaacgcctcagcctagttcctagccacagaccaagcagaaccaccaaaccacgccgagcgaggagatggggcaagaggacgggggagacgatcgccgtaccttgaagcgggggaaggatcgccgagggtcgcgaggagagcaatttggatttggaacccgggggttgtgcgctccgaacgatgagacgatgtgagattgtgggaagaggcgcggagggccctgtatttatgggctgcgacggggggaggagaggtggggagggttggggaaggaatcccccacccgtgccgtgacggttccgggccgtgtgagaggagcccgctcgtctccgccacgcaatttccgcgatcggagcggagctttcgagaggcggctggatggttggtggccgttagatttgtagacgccgttaacgcctcgcctccaccgggaagagttttgagcagccgcttatgacaatggcttaacgacgttagacggagcgttagtggcaggccatgatagaatagacgtattgcaatgggattataattaaataaataagaatataataagatatggcaagtcggcactcatgacatggtcttcgaaatgatagtgctcactttcttagccgagaaagttgacgcgactgatttagaagttaagatattatttctctcttcttttctttcctcgtcatataaggatgaaataaactttagagattgccggtagtgattttggatttcggcgatcaggcttggtttgccggtttcggacggtgtgccttaggccacccgcagtgtatcttgtaatgttcaaccgataagcaagggtggggctcaagcaagtagtaaacaactatgtcaaatgtcaccatggttatggtcttgtttagttggcttcttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatctgcaggcgcgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgaaatgtacgcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctagcgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttcctgcagggcgatctattcgaatgcctaagaagaagagaaaggtcggtattcacggcgttcctgcggcgagtgacaagaagtactcgatcggcctcgacatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctgaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagtgactcgagatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataaggagctccggtgacggacgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgatgaggaactggaccaagggttttagagctagaaatagcaagttaaaataaggctagtccgttatcaacttgaaaaagtggcaccgagtcggtgctttttttcccggtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcg

SEQ ID NO: 36 - DNA, 19778 bp, Artificial Sequence
  Synthetic construct
actgacagaaccgcaacgttgaaggagccactcagcaagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgagatgtaccgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagccgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcactagtaacggccgccagtgtgctggaattcgccctttaaataagcgatcgccccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccccgatccctacagggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgatagttacagaaccgtcgagggggatccaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatttaaattgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgggcgcggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttgga

SEQ ID NO: 37 - DNA, 1107 bp, Triticum aestivum
atgctgagctcgtcggcggcgatggggatggggctgggcgggtacggccagcagcagcagcagcagcagatgcagatgcagatgcagcggggggcggggccggtgttcacgccggcgcagtgggccgagctggagcagcaggcgctgatttacaagtacctcatggcgggcgtgcccgtgccgcccgatctcctgctccccatccgcccccaccccgccggcgccggagccaccttctccttcgccaaccccgccgcctcgcccttctaccaccaccaccacccctccatgagttactacgcctactatggcaagaagctcgacccggagccgtggcggtgccgccgcaccgacggcaagaagtggaggtgctccaaggaggcgcaccccgactccaagtactgcgagcgccacatgcaccgtggccgcaaccgttcaagaaagcctgtggaatccaagtctgcttcccctgcgcaccagtcgcagcagcccccgttgtccgccgtcacgtccgccacccgcgacgccgagcctctcccctccctcccggcgggggctaagacccatggcctgtccctcggcggggctggctcgtcgcagatgcacgtcgacgcctcgtcatacggcaacaaatactcccttggagctaaatctgacgtgggtgaactgagcttcttctctggagcatcaggaaacaacaacaggggcttcaccatcgattccccaacggacagctcgtggcactcaatgggatccagcctgcccccgtaccaactgtcgaaacctagagattccggcctcatgcaaggcggcttctcgtattcccactttgagccgtcgcaggagcttgggcaggtaaccatcgcctcgctgtcccactcccaggagcaggaccgccgctccttcggtggcggcggtggtggtggaggtggaggggcagggctcatgggaaatgttaagcaggagaaccagccgctgaggcccttcttcgacgagtggccggggaggcgggactcgtggtcggagatggacgacgagcgctccaacggcacctccttctcgacgacccagctctcgatctccatcccaatgcctcgatgtgattga

SEQ ID NO: 38 - DNA, 1230 bp, Triticum aestivum
atgatgatgatgggcggtcgcgcgggggccggcggcgtcggggcaggcggcggccggtgcccgttcacggcgacgcagtggcaggagctggagcaccaggcgctcatctacaagtacatggcctccggcgtgcccatcccctccgacctcctcctcccgctccgccgcagcttcctcctcgactccgccctcgccacctccccctccctcgccttccctccccaggccgcactgggctggggatgtttcgggatggggttcggccggaaggcggaggacccggagccggggcggtgccggcggactgacggcaagaagtggcgctgctccaaggaggcgtacccggactccaagtactgcgagaagcacatgcaccggggcaagaaccgttcaagaaagcctgtggaaatgtccttggccacgcccccgccgccgccttcctcctcggcctcctcttcctcctccaacgtccactccgccgtcaacgtcgccaccaccaccacctccccagcgccgtcctaccaccgccacgccgctgcgactcacgacacgacgccctaccacgccctctacggcggcccctactcctccgccggccgccagcagcacgccagcgcctaccaccacgcggcgcaggtcagcccgttccacctgcacctcgacaccacccacccgcacccgccgccgtcctactactccaccatggaccacagcaaggacagctacgcctacgggcacagcgtcaaggaggtgcacggcggcggcgagcacgccttcttctcctccgacgtcagcaccgacagggaccaccaccaccatcagcaccaacaccacgctagcgccggcggcaacggccagtggcagttcaagcagctcggcggcatggagcccaagcagcacaaccccacgtcgctcttccccggctacggcaacaacgcggcgtacgccatcgacctgtccagcaaagaagaggacgaggagaaggagaggcggcaacagcagcagcactgcttcctgctgggcgccgacctgaggctcgacaagccgtcgtcggggcacggcgactccgccgaccagaagcctctccggccgttcttcgacgagtggccgcacgagaagactggcagcaaggggtcgtggatggggctcgagggggagacgcagctctccatctccatcgccaatgaactccccatcaccaccacctcccgctaccaccatggtgaatga

SEQ ID NO: 39 - DNA, 1227 bp, Triticum aestivum
atggagctcgggcaggtgctgggctacacgccaccggcgaccaaggacgcgagatccggcggcggcttcacccaggctgccgcttgcccctacccctacccctcccccttcctcgacgagcagaagatgctcagcttctccaaggccgccgccgctcaccagccgccctcaggtatggattttgggaggtccaatgagcagaggctgttgctggccaggagcaagatgcccttcaccccttcacagtggatggagctggagcaccaggccctcatttacaagtatctcaatgcaaaggcccccataccttccggcctgctcatctccatcagcaagagcttcagaccctcctccgatagaatgccctggaggcctgtctatcaagggttcaccaatgcagattctgacccggaacctggaagatgccgtcgaacagacggcaagaaatggcggtgctcaaaggaggcgatggccgagcacaagtactgtgagcggcacatcaataggaaccgccatcgttcaagaaagcctgtggaaaaccaaacaaggaagaacgccaaagagacacctgctgctggctcgatatcggccgctgtctcacagggtggctgtaagaaagcaaaagctggtgatgaactgaagccagggagcgtcagttattggacagataatttaaacagggcaatggtgagcaaagccaggggaaacaaccctgaagaaggcaacagtgctccactcctgaattctactaatcaacaacacacattgtccttgttctctcaactgaagcaacagagcaaaccagataagttcagcccggcagtcgatagtgaatcgatctcctcaaatactgtattgaagccttgggaaagaagcaaccagcagagcagtaaggacgtttcttccacgacgctccatgatcgcgggtgccttcaatcagtccttcaagatttcagcatgcataagaatgacaagatcgagtctcagaaaaacaatgcttcagtgccatctactttctattcatctacagaaggtcgacacatcagctgccttgcatctaacatgatgcaagtgcaggaggattgcatctcaagctcttgggagatacctcaaggtgggcctttaggtgaaatcctaacaaactccaagaatactgatgacttgaccaataagtgtgaatcaagatcatatggttggttactgagtcttgatgaacatgaaatgtga

SEQ ID NO: 40 - DNA, 1917 bp, Artificial Sequence
  Synthetic construct
atgatgatgatgggcggtcgcgcgggggccggcggcgtcggggcaggcggcggccggtgcccgttcacggcgacgcagtggcaggagctggagcaccaggcgctcatctacaagtacatggcctccggcgtgcccatcccctccgacctcctcctcccgctccgccgcagcttcctcctcgactccgccctcgccacctccccctccctcgccttccctccccaggccgcactgggctggggatgtttcgggatggggttcggccggaaggcggaggacccggagccggggcggtgccggcggactgacggcaagaagtggcgctgctccaaggaggcgtacccggactccaagtactgcgagaagcacatgcaccggggcaagaaccgttcaagaaagcctgtggaaatgtccttggccacgcccccgccgccgccttcctcctcggcctcctcttcctcctccaacgtccactccgccgtcaacgtcgccaccaccaccacctccccagcgccgtcctaccaccgccacgccgctgcgactcacgacacgacgccctaccacgccctctacggcggcccctactcctccgccggccgccagcagcacgccagcgcctaccaccacgcggcgcaggtcagcccgttccacctgcacctcgacaccacccacccgcacccgccgccgtcctactactccaccatggaccacagcaaggacagctacgcctacgggcacagcgtcaaggaggtgcacggcggcggcgagcacgccttcttctcctccgacgtcagcaccgacagggaccaccaccaccatcagcaccaacaccacgctagcgccggcggcaacggccagtggcagttcaagcagctcggcggcatggagcccaagcagcacaaccccacgtcgctcttccccggctacggcaacaacgcggcgtacgccatcgacctgtccagcaaagaagaggacgaggagaaggagaggcggcaacagcagcagcactgcttcctgctgggcgccgacctgaggctcgacaagccgtcgtcggggcacggcgactccgccgaccagaagcctctccggccgttcttcgacgagtggccgcacgagaagactggcagcaaggggtcgtggatggggctcgagggggagacgcagctctccatctccatcgccaatgaactccccatcaccaccacctcccgctaccaccatggtgaagcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctag

SEQ ID NO: 41 - DNA, 1794 bp, Artificial Sequence
  Synthetic construct
atgctgagctcgtcggcggcgatggggatggggctgggcgggtacggccagcagcagcagcagcagcagatgcagatgcagatgcagcggggggcggggccggtgttcacgccggcgcagtgggccgagctggagcagcaggcgctgatttacaagtacctcatggcgggcgtgcccgtgccgcccgatctcctgctccccatccgcccccaccccgccggcgccggagccaccttctccttcgccaaccccgccgcctcgcccttctaccaccaccaccacccctccatgagttactacgcctactatggcaagaagctcgacccggagccgtggcggtgccgccgcaccgacggcaagaagtggaggtgctccaaggaggcgcaccccgactccaagtactgcgagcgccacatgcaccgtggccgcaaccgttcaagaaagcctgtggaatccaagtctgcttcccctgcgcaccagtcgcagcagcccccgttgtccgccgtcacgtccgccacccgcgacgccgagcctctcccctccctcccggcgggggctaagacccatggcctgtccctcggcggggctggctcgtcgcagatgcacgtcgacgcctcgtcatacggcaacaaatactcccttggagctaaatctgacgtgggtgaactgagcttcttctctggagcatcaggaaacaacaacaggggcttcaccatcgattccccaacggacagctcgtggcactcaatgggatccagcctgcccccgtaccaactgtcgaaacctagagattccggcctcatgcaaggcggcttctcgtattcccactttgagccgtcgcaggagcttgggcaggtaaccatcgcctcgctgtcccactcccaggagcaggaccgccgctccttcggtggcggcggtggtggtggaggtggaggggcagggctcatgggaaatgttaagcaggagaaccagccgctgaggcccttcttcgacgagtggccggggaggcgggactcgtggtcggagatggacgacgagcgctccaacggcacctccttctcgacgacccagctctcgatctccatcccaatgcctcgatgtgatgcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctag

SEQ ID NO: 42 - DNA, 1914 bp, Artificial Sequence
  Synthetic construct
atggagctcgggcaggtgctgggctacacgccaccggcgaccaaggacgcgagatccggcggcggcttcacccaggctgccgcttgcccctacccctacccctcccccttcctcgacgagcagaagatgctcagcttctccaaggccgccgccgctcaccagccgccctcaggtatggattttgggaggtccaatgagcagaggctgttgctggccaggagcaagatgcccttcaccccttcacagtggatggagctggagcaccaggccctcatttacaagtatctcaatgcaaaggcccccataccttccggcctgctcatctccatcagcaagagcttcagaccctcctccgatagaatgccctggaggcctgtctatcaagggttcaccaatgcagattctgacccggaacctggaagatgccgtcgaacagacggcaagaaatggcggtgctcaaaggaggcgatggccgagcacaagtactgtgagcggcacatcaataggaaccgccatcgttcaagaaagcctgtggaaaaccaaacaaggaagaacgccaaagagacacctgctgctggctcgatatcggccgctgtctcacagggtggctgtaagaaagcaaaagctggtgatgaactgaagccagggagcgtcagttattggacagataatttaaacagggcaatggtgagcaaagccaggggaaacaaccctgaagaaggcaacagtgctccactcctgaattctactaatcaacaacacacattgtccttgttctctcaactgaagcaacagagcaaaccagataagttcagcccggcagtcgatagtgaatcgatctcctcaaatactgtattgaagccttgggaaagaagcaaccagcagagcagtaaggacgtttcttccacgacgctccatgatcgcgggtgccttcaatcagtccttcaagatttcagcatgcataagaatgacaagatcgagtctcagaaaaacaatgcttcagtgccatctactttctattcatctacagaaggtcgacacatcagctgccttgcatctaacatgatgcaagtgcaggaggattgcatctcaagctcttgggagatacctcaaggtgggcctttaggtgaaatcctaacaaactccaagaatactgatgacttgaccaataagtgtgaatcaagatcatatggttggttactgagtcttgatgaacatgaaatggcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctag

SEQ ID NO: 43 - DNA, 558 bp, Triticum aestivum
atgcagcaagcgatgcccatgccgccggcggcggcggcgccggggatgcctccatctgctggcctcagcaccgagcagatccaaaagtacctggatgaaaataagcaactaattttggctatcttggaaaatcagaacctgggaaagttggcggaatgtgctcagtatcaagctcagcttcagaagaatcttttgtatttggctgcaattgctgatactcagccacagacctctgtaagccgtcctcagatggcaccacctagtgcatccccaggggtagggcattacatgtcacaggtgccaatgttccctccaaggacccctctaacgcctcagcagatgcaggagcagcaactacagcaacaacaggctcagatgcttccgtttgctggtcaaatggttgcgagacctggggctgtgaatggcatggcccgggcccctcaagttgaaccagcctatgcagcaggtggggccagttccgagccttctggcactgagagccataggagcactggtgccgataacaacggtggaagcggcttggctgaccagtcctaa

SEQ ID NO: 44 - DNA, 606 bp, Triticum aestivum
atgcagcaggcgatgtccttgcccccgggagcggtcggcgcggtgtccccgccggccggcatcaccaccgagcagatccaaaagtatttggatgaaaataagcaacttattttggccatccttgaaaatcagaacctaggaaagttggctgaatgtgctcaatatcaagctcagctccaaaagaatctcttgtatctagctgctatcgcggatgcccaaccaccacagaaccctgcaagtcgcccccagatggtgcagcagcctggtagtatgcaaggtgcagggcattacatgtcacaagtaccaatgttccctccaagaacgcctttaaccccacagcagatgcaagagcagcagcaccagcagcttcagcagcagcaagcccaggccctttctttccccgcccaggtggtcatgagaccaggcaccgtcaacggcatgcagcagcctatgcaagcagccggcgacctccagccagcagcagcacctggagggagcaagcaggacgccgcagtggctggggccagctcggaaccatctggcaccaagagccacaagaacgcgggagcagaggaggtgggcgctgatgtagcagaacaatcctaa

SEQ ID NO: 45 - DNA, 1791 bp, Artificial Sequence
  Synthetic construct
atggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgagatgtacgcggccgctgccatgcagcaagcgatgcccatgccgccggcggcggcggcgccggggatgcctccatctgctggcctcagcaccgagcagatccaaaagtacctggatgaaaataagcaactaattttggctatcttggaaaatcagaacctgggaaagttggcggaatgtgctcagtatcaagctcagcttcagaagaatcttttgtatttggctgcaattgctgatactcagccacagacctctgtaagccgtcctcagatggcaccacctagtgcatccccaggggtagggcattacatgtcacaggtgccaatgttccctccaaggacccctctaacgcctcagcagatgcaggagcagcaactacagcaacaacaggctcagatgcttccgtttgctggtcaaatggttgcgagacctggggctgtgaatggcatggcccgggcccctcaagttgaaccagcctatgcagcaggtggggccagttccgagccttctggcactgagagccataggagcactggtgccgataacaacggtggaagcggcttggctgaccagtcctaa

SEQ ID NO: 46 - DNA, 1839 bp, Artificial Sequence
  Synthetic construct
atggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgagatgtacgcggccgctgccatgcagcaggcgatgtccttgcccccgggagcggtcggcgcggtgtccccgccggccggcatcaccaccgagcagatccaaaagtatttggatgaaaataagcaacttattttggccatccttgaaaatcagaacctaggaaagttggctgaatgtgctcaatatcaagctcagctccaaaagaatctcttgtatctagctgctatcgcggatgcccaaccaccacagaaccctgcaagtcgcccccagatggtgcagcagcctggtagtatgcaaggtgcagggcattacatgtcacaagtaccaatgttccctccaagaacgcctttaaccccacagcagatgcaagagcagcagcaccagcagcttcagcagcagcaagcccaggccctttctttccccgcccaggtggtcatgagaccaggcaccgtcaacggcatgcagcagcctatgcaagcagccggcgacctccagccagcagcagcacctggagggagcaagcaggacgccgcagtggctggggccagctcggaaccatctggcaccaagagccacaagaacgcgggagcagaggaggtgggcgctgatgtagcagaacaatcctaa

SEQ ID NO: 47 - DNA, 15957 bp, Artificial Sequence
  Synthetic construct
aagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagactagagttatcaacaagtttgtacaaaaaagctgccaccatggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgaaatgtacgcggccgctgccatgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagctagcgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcactagtaacggccgccagtgtgctggaattcgccctttaaataagcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatttaaattgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgggcgcggggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactaggtcgctaccttaggaccgttatagttactagcgaattgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaaggatcttctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcactctagtgatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgccagatccaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggctgctcgactgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgattcgatagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgccagccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagc

SEQ ID NO: 48 - DNA, 1671 bp, Artificial Sequence
  Synthetic construct
atgaatagcggcaacggcggtggcggtgctggtggggctggtgggttggggatgatgggaatgagggcatcaccttttacggtatcacaatggcaagaactggaacatcaagctttgatctttaagtacatggtggcaggtttgcctgtgccacctgatcttgtactccctatacaaaagagctttgagtctatttctcataggttctttcatcaccccaccatgggttattgttccttctatgggaaaaaagttgatcccgaaccaggacggtgcaggaggactgacggcaaaaaatggcggtgttccaaagatgcatacccagactcaaagtactgtgagcgccacatgcaccgcggccgcaaccgttcaagaaagcctgtggaatcacaaaccataacacagtcatcatccactgtgacatcactgactgccactggaagcagtggtgggactggaagctaccagaaccttccattacatgcctttgctaatccccaaggcactacttctggaaccaaccaatcccattatcatgtggactccatttcctgtggaattcccaacaaagaatacaggtaccttcaagttcaaggacttaaacccgaggttggagagcatagtttcttcactgaagcttcaggaagcaataggggtctccagatggactcaccaattgacagtgcatggcctctgatgcaatctagagtctcctcgcatccccagtcgaaatccagcaacacctccattttgcagaatgattatcctcagcattcacttttcagtagtgaattcacctcgggtgagcctgtaaaacaggaaggtcagtctctccgacctttctttgatgagtggcctaagacaagagactcatggtctggtcttgaagacaagagatcccaccagacctcattctcaacaaccaagctctctatatccatccctatggcctcatccgacttctcggcaacgagttctcgatcgcctcatgataacgcggccgctgccatgcaacagcacctgatgcagatgcagcccatgatggcagcttattatcccaacaacgtcactactgaccacattcaacagtatctagatgagaacaaatcattgattttgaagattgttgagagccagaattcagggaaactgagcgagtgtgcagagaaccaggcaagattgcagcggaatctcatgtacctggctgctattgctgatgctcaaccccaaccacctagcgttcatgcccagttctcttctggtggcattatgcagccaggagctcactatatgcaacaccagcaatctcagccaatgacaccacagtcacttatggctgcacgctcatccatggtgtactctcaacagcaattttcagtgcttcagcaacagcaagccttgcatggtcagcttggcatgagctctggtggtagctcaggacttcacatgctgcaaagtgagggtagtactgcaggaggtagtggttcacttgggggtgggggattccctgattttggccgtggctcatctggtgaaggcttgcactcaaggggaatggggagcaagcatgatataggcagttctggatctgctgaaggacgaggagggagctcaggaagccaagatggaggcgaaactctctacttgaaaggggctgatgatggaaattaa

SEQ ID NO: 49 - DNA, 17363 bp, Artificial Sequence
  Synthetic construct
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagttatcaacaagtttgtacaaaaaagctgccaccatgaatagcggcaacggcggtggcggtgctggtggggctggtgggttggggatgatgggaatgagggcatcaccttttacggtatcacaatggcaagaactggaacatcaagctttgatctttaagtacatggtggcaggtttgcctgtgccacctgatcttgtactccctatacaaaagagctttgagtctatttctcataggttctttcatcaccccaccatgggttattgttccttctatgggaaaaaagttgatcccgaaccaggacggtgcaggaggactgacggcaaaaaatggcggtgttccaaagatgcatacccagactcaaagtactgtgagcgccacatgcaccgcggccgcaaccgttcaagaaagcctgtggaatcacaaaccataacacagtcatcatccactgtgacatcactgactgccactggaagcagtggtgggactggaagctaccagaaccttccattacatgcctttgctaatccccaaggcactacttctggaaccaaccaatcccattatcatgtggactccatttcctgtggaattcccaacaaagaatacaggtaccttcaagttcaaggacttaaacccgaggttggagagcatagtttcttcactgaagcttcaggaagcaataggggtctccagatggactcaccaattgacagtgcatggcctctgatgcaatctagagtctcctcgcatccccagtcgaaatccagcaacacctccattttgcagaatgattatcctcagcattcacttttcagtagtgaattcacctcgggtgagcctgtaaaacaggaaggtcagtctctccgacctttctttgatgagtggcctaagacaagagactcatggtctggtcttgaagacaagagatcccaccagacctcattctcaacaaccaagctctctatatccatccctatggcctcatccgacttctcggcaacgagttctcgatcgcctcatgataacgcggccgctgccatgcaacagcacctgatgcagatgcagcccatgatggcagcttattatcccaacaacgtcactactgaccacattcaacagtatctagatgagaacaaatcattgattttgaagattgttgagagccagaattcagggaaactgagcgagtgtgcagagaaccaggcaagattgcagcggaatctcatgtacctggctgctattgctgatgctcaaccccaaccacctagcgttcatgcccagttctcttctggtggcattatgcagccaggagctcactatatgcaacaccagcaatctcagccaatgacaccacagtcacttatggctgcacgctcatccatggtgtactctcaacagcaattttcagtgcttcagcaacagcaagccttgcatggtcagcttggcatgagctctggtggtagctcaggacttcacatgctgcaaagtgagggtagtactgcaggaggtagtggttcacttgggggtgggggattccctgattttggccgtggctcatctggtgaaggcttgcactcaaggggaatggggagcaagcatgatataggcagttctggatctgctgaaggacgaggagggagctcaggaagccaagatggaggcgaaactctctacttgaaaggggctgatgatggaaattaacgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattagcttcatcaacgcaagacatgcgcacgaccgtctgacaggagaggaatttccgacgagcacagaaaggacttgctcttggacgtaggcctatttctcaggcacatgtatcaagtgttcggacgtgggttttcgatggtgtatcagccgccgccaactgggagatgaggaggctttcttggggggcagtcagcagttcatttcacaagacagaggaacttgtaaggagatgcactgatttatcttggcgcaaaccagcaggacgaattagtgggaatagcccgcgaatatctaagttatgcctgtcggcatgagcagaaacttccaattcgaaacagtttggagaggttgtttttgggcataccttttgttagtcagcctctcgattgctcatcgtcattacacagtaccgaagtttgatcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcgacgcactccttctttactccaccatctcgtccttattgaaaacgtgggtagcaccaaaacgaatcaagtcgctggaactgaagttaccaatcacgctggatgatttgccagttggattaatcttgcctttccccgcatgaataatattgatgaatgcatgcgtgaggggtatttcgattttggcaatagctgcaattgccgcgacatcctccaacgagcataattcttcagaaaaatagcgatgttccatgttgtcagggcatgcatgatgcacgttatgaggtgacggtgctaggcagtattccctcaaagtttcatagtcagtatcatattcatcattgcattcctgcaagagagaattgagacgcaatccacacgctgcggcaaccttccggcgttcgtggtctatttgctcttggacgttgcaaacgtaagtgttggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcggggtcgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacggatctctaggacgcgtcctagaagctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 50 - DNA, 1698 bp, Artificial Sequence
  Synthetic construct
atgaagcaaagctttgtgggggtttcttcacaaacagaggctgagacaagagccagagagagtgacacacagacacacccacaaaggaacctcagtcactgtccccaaaaacatcctcaaaacttgcaaagaacccaaaatggtgggtctgttgggggggcatcaatggcaacagggatgatggtgaggtcatcaccattcacagtgtctcagtggcaggagctggagcatcaagctttgatctttaagtatctaatggcaggactacccgtcccacctgatcttgtgcttcctattcagaagagctttgaatctatgtcttctaggttcttccaccatcctaccatgggttactgttccttctatgggaagaaggtggacccggagccaggtagatgccggaggactgatggcaagaaatggaggtgctccaaagacgcctacccggactccaagtactgtgagcgccacatgcaccgtggccgcaaccgttctagaaaaccggtcgaatcacaaactactacacagtcttcttcgactgtgacttcactaactgtcactggaggcagtggcagtggagccgggaccttccagaaccttcccctgcaaaacttcaataacccccaaggcggcccggggtccggaaccaaccaatcccaattccatatggagcccattccctatggcattcctaataaagattacaggtatcttcatggacttaaacccgaggtaggcgaacatagtttcttctctgaagcttcaggaagcaacaggggtcttcagatggactcgcctttggacagcacatggcctctgatgccatcacgggtctcatcattccccccatcaaaatcaagtgaaaactccatcttgcagagtgactacccccagcattcgttcttcagcagcgaatttgcgtcgggagaggttgtgaaacaggagggtcagtctctccgtcctttctttgacgagtggcctaaaaccagagactcctggtccggccttgaggaagagaggtccaaccagacctcattctccacaacccagctctcaatttcgatcccaatggcctcctctgacttctcaacaaccagctctcggtctccccacgataacgcggccgctgccatgcagcccatgatggcagcctattaccccagcaacgtcaccactgatcacattcagcagtatcttgatgaaaacaagtcattgattctgaagattgttgagagccagaattcaggaaaattgactgaatgtgcagagaaccaggcaagactacagagaaacctcatgtacctggctgcaatcgctgattctcaaccccaaccacccaccatgcatgctcagttccctcctagtggcattgttcagccaggagctcactacatgcagcaccaacaagctcaacaaatgacaccacagtcgctcctggctgcacgctcctccatgctgtactcccaacaaccattttcggccctgcaacaacaacaagccatccatagccagcttggcatgggctctggtggaagtgcaggacttcacatgctgcaaagcgaggggagtaatccaggaggcaatggaacactggggactgatatgggaaatgcagaagggcgaggagggaactcaggaggtcagggtggggatggaggtgagactctttacttgaaagctgctgaagatgggaattga

SEQ ID NO: 51 - DNA, 1383 bp, Triticum aestivum
atggcgatgccgtatgcctctctttccccggcaggcgaccgccgctcctccccggccgccaccgccaccgcctccctcctccccttctgccgctcctcccccttctccgccggcggcaatggcggcatgggggaggaggcgcggatggacgggaggtggatggcgaggccggtgcccttcacggcggcgcagtacgaggagctggagcaccaggcgctcatatacaagtacctggtggccggcgtgtccgtcccgccggatctcgtgctccccatccgccgcggcatcgagtccctcgccgcccgcttctaccacaaccccctcgccatcgggtacggatcgtacctgggcaagaaggtggatccggagccgggccggtgccggcgcacggacggcaagaagtggcggtgcgccaaggaggccgcctccgactccaagtattgcgagcgccacatgcaccgcggccgcaaccgttcaagaaagcctgtggaaacgcagctcgtctcgcactcccagccgccggccgcctccgtcgtgccgcccctcgccaccggcttccacaaccactccctctaccccgccatcggcggcaccaacggtggtggaggcggggggaacaacggcatgtccatgcccggcacgttctcctccgcgctggggccgcctcagcagcacatgggcaacaatgccgcctctccctacgcggctctcggcggcgccggaacatgcaaagatttcaggtataccgcatatggaataagatctttggcagacgagcagagtcagctcatgacagaagccatgaacacctccgtggagaacccatggcgcctgccgccatcttctcaaacgactacattcccgctctcaagctactctcctcagcttggagcaacgagtgacctgggtcagaacaacagcagcaacaacaacagcggcgtcaaggccgagggacagcagcagcagcagccgctctccttcccggggtgcggcgacttcggcagcggcgactccgcgaagcaggagaaccagacgctgcggccgttcttcgacgagtggccgaagacgagggactcgtggtcggacctgaccgacgacaactcgaacgtcgcctccttctcggccacccagctgtcgatctcgatacccatgacgtcctccgacttctccgccgccagctcccagtcgcccaacggcatgctgttcgccggcgagatgtaccgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttag

SEQ ID NO: 52 - DNA, 837 bp, Triticum aestivum
atgcagcagcaacacctgatgcagatgaaccagagcatgatggggggctacgcttcctctaccactgccaccactgatctcattcagcagtacctggatgagaacaagcagctgatcctggccatcctcgacaaccagaacaacggcaaggtggaggagtgcgcacggaaccaagctaagctccagcagaacctcatgtacctcgccgccatcgccgacagccagcctccgcagacggcatcgctgtctcagtacccgtccaacctgatgatgcagtccgggccgcggtacatgcagcagcagtcggcgcagatgatgtcgccgcagtcgctgatggcggcgcggtcgtcgatgatgtacgcgcagcaggccatgtcgccgctccagcagcagcagcagcagcagcagcaccaggcggccgcgcacggccagctggggatgtcctccggcgcgaccaccgggttcaacctcctgcacggtgaggccagcatgggcggcggcggcggcgccactggcaacagcatgatgaacgccagcgtcttctcggactatggccgcggcggcagcggcgccaaggaggggtcgacctcgctgtcggccgacgctcgcggcgccaactctggcgcgcacagcggcgacggggagtacctcaagggcaccgaggaggaaggaagccgttcagctttcttgtacaaagtggttgataacagcgggttaattaacatcttttacccatacgatgttcctgactatgcgggctatccctatgacgtcccggactatgcaggatcctatccatatgacgttccagattacgctgctcagtgcagcgcttag

SEQ ID NO: 53 - DNA, 42 bp, Artificial Sequence
  Synthetic construct
gcggccgcaaccgttcaagaaagcctgtggaaacgcagctcg

SEQ ID NO: 54 - DNA, 42 bp, Artificial Sequence
  Synthetic construct
gcggccgcaaccgttctagaaaaccggtcgaaacgcagctcg

SEQ ID NO: 55 - DNA, 21 bp, Artificial Sequence
  Synthetic construct
gtcaagttctttcggacacct

SEQ ID NO: 56 - protein, 7 aa, Artificial Sequence
  Synthetic construct
RSRKLVE

SEQ ID NO: 57 - DNA, 25 bp, Artificial Sequence
  Synthetic construct
gtttacaccacaatatatcctgcca

SEQ ID NO: 58 - DNA, 25 bp, Artificial Sequence
  Synthetic construct
gtttacccgccaatatatcctgtca

SEQ ID NO: 59 - DNA, 1992 bp, Artificial Sequence
  Synthetic construct
aagctttgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 60 - DNA, 120 bp, Artificial Sequence
  Synthetic construct
gggttaattaacatcttttacccatacgatgttcctgactatgcgggctatccctatgacgtcccggactatgcaggatcctatccatatgacgttccagattacgctgctcagtgcagc

SEQ ID NO: 61 - DNA, 253 bp, Artificial Sequence
  Synthetic construct
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 62 - DNA, 755 bp, Artificial Sequence
  Synthetic construct
ccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggac

SEQ ID NO: 63 - DNA, 1038 bp, Artificial Sequence
  Synthetic construct
atgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtag

SEQ ID NO: 64 - DNA, 4203 bp, Artificial Sequence
  Synthetic construct
atgcctaagaagaagagaaaggtcggtattcacggcgttcctgcggcgagtgacaagaagtactcgatcggcctcgacatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctgaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagtga

SEQ ID NO: 65 - DNA, 362 bp, Artificial Sequence
  Synthetic construct
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagactt

SEQ ID NO: 66 - DNA, 20 bp, Artificial Sequence
  Synthetic construct
gatgaggaactggaccaagg

SEQ ID NO: 67 - DNA, 51 bp, Artificial Sequence
  Synthetic construct
gttttagagctagaaatagcaagttaaaataaggctagtccgttatcaact

SEQ ID NO: 68 - DNA, 40 bp, Artificial Sequence
  Synthetic construct
gttttttgccagatgaggaactggaccaaggaggagttcg

SEQ ID NO: 69 - protein, 35 aa, Artificial Sequence
  Synthetic construct
PFTATQWQELEHQALIYKYMASGVPIPSDLLLPLR

SEQ ID NO: 70 - protein, 35 aa, Artificial Sequence
  Synthetic construct
LFTASQWRELEHQALIYKYMAAGSQVPHELVLPLR

SEQ ID NO: 71 - protein, 35 aa, Artificial Sequence
  Synthetic construct
TFTAAQYEELEQQALIYKYLVAGVPVPPDLLLPIR

SEQ ID NO: 72 - protein, 35 aa, Artificial Sequence
  Synthetic construct
PFTAAQYEELEHQALIYKYLVAGVSVPPDLVLPIR

SEQ ID NO: 73 - protein, 35 aa, Artificial Sequence
  Synthetic construct
VFTPAQWAELEQQALIYKYLMAGVPVPPDLLLPIR

SEQ ID NO: 74 - protein, 35 aa, Artificial Sequence
  Synthetic construct
PFTASQWQELEHQALIYKYMASGTPIPSDLILPLR

SEQ ID NO: 75 - protein, 35 aa, Artificial Sequence
  Synthetic construct
LFTATQWQELEHQALIYKYMAAGAPVPPDLLLHLR

SEQ ID NO: 76 - protein, 35 aa, Artificial Sequence
  Synthetic construct
PFTAAQYEELEQQALIYKYLVAGVPVPADLLLPIR

SEQ ID NO: 77 - protein, 36 aa, Artificial Sequence
  Synthetic construct
PPFTAAQYEELEQQALIYKYLVAGVPVPPDLVLPIR

SEQ ID NO: 78 - protein, 35 aa, Artificial Sequence
  Synthetic construct
VFTAAQWAELEQQALIYKYLVAGVPVPGDLLLPIR

SEQ ID NO: 79 - protein, 35 aa, Artificial Sequence
  Synthetic construct
FFSWAQWQELELQALIYRYMLAGAAVPQELLLPIK

SEQ ID NO: 80 - protein, 35 aa, Artificial Sequence
  Synthetic construct
FFSWAQWQELELQALIYRYMLAGASVPQELLLPIK

SEQ ID NO: 81 - protein, 36 aa, Artificial Sequence
  Synthetic construct
PFTPTQWEELEHQALIYKYMVSGVPVPPELIFSIRR

SEQ ID NO: 82 - protein, 35 aa, Artificial Sequence
  Synthetic construct
PFTESQWEELENQALVFKYLAANMPVPPHLLFLIK

SEQ ID NO: 83 - protein, 36 aa, Artificial Sequence
  Synthetic construct
PFTVSQWQELEHQALIFKYLMAGLPVPPDLVLPIQK

SEQ ID NO: 84 - protein, 44 aa, Artificial Sequence
  Synthetic construct
DPEPGRCRRTDGKKWRCSKEAYPDSKYCEKHMHRGKNRSRKPVE

SEQ ID NO: 85 - protein, 44 aa, Artificial Sequence
  Synthetic construct
DPEPGRCRRTDGKKWRCSREAYGESKYCDRHMHRGKNRSRKPVE

SEQ ID NO: 86 - protein, 44 aa, Artificial Sequence
  Synthetic construct
DPEPGRCRRTDGKKWRCSKEAAQDSKYCERHMHRGRNRSRKPVE

SEQ ID NO: 87 - protein, 44 aa, Artificial Sequence
  Synthetic construct
DPEPGRCRRTDGKKWRCAKEAASDSKYCERHMHRGRNRSRKPVE

SEQ ID NO: 88 - protein, 44 aa, Artificial Sequence
  Synthetic construct
DPEPWRCRRTDGKKWRCSKEAHPDSKYCERHMHRGRNRSRKPVE

SEQ ID NO: 89 - protein, 44 aa, Artificial Sequence
  Synthetic construct
DPEPGRCRRTDGKKWRCSREAYGESKYCEKHMHRGKNRSRKPVE

SEQ ID NO: 90 - protein, 44 aa, Artificial Sequence
  Synthetic construct
DPEPGRCRRTDGKKWRCSKEAAPDSKYCERHMHRGRNRSRKPVE

SEQ ID NO: 91 - protein, 44 aa, Artificial Sequence
  Synthetic construct
DPEPGRCRRTDGKKWRCSRDVFAGHKYCERHMHRGRNRSRKPVE

SEQ ID NO: 92 - protein, 44 aa, Artificial Sequence
  Synthetic construct
DPEPGRCKRTDGKKWRCSRDVVAGHKYCDRHIHRGRNRSRKPVE

SEQ ID NO: 93 - protein, 44 aa, Artificial Sequence
  Synthetic construct
DPEPGRCRRTDGKKWRCSREAYPDSKYCEKHMHRGRNRARKSLD

SEQ ID NO: 94 - protein, 44 aa, Artificial Sequence
  Synthetic construct
DAEPGRCRRTDGKKWRCSKEAYPDSKYCERHMHRGKNRSSSRKP

SEQ ID NO: 95 - protein, 44 aa, Artificial Sequence
  Synthetic construct
DPEPGRCRRTDGKKWRCSKDAYPDSKYCERHMHRGRNRSRKPVE

SEQ ID NO: 96 - protein, 79 aa, Artificial Sequence
  Synthetic construct
PFTAAQYEELEHQALIYKYLVAGVSVPPDLVLPIRDPEPGRCRRTDGKKWRCAKEAASDSKYCERHMHRGRNRSRKPVE

SEQ ID NO: 97 - protein, 59 aa, Artificial Sequence
  Synthetic construct
SNVTSDHIQQYLDENKSLILKIVESQNSGKLSECAENQARLQRNLMYLAAIADSQPQPP

SEQ ID NO: 98 - protein, 59 aa, Artificial Sequence
  Synthetic construct
NNITTEQIQKYLDENKKLIMAIMENQNLGKLAECAQYQALLQKNLMYLAAIADAQPPPP

SEQ ID NO: 99 - protein, 59 aa, Artificial Sequence
  Synthetic construct
NNITTEQIQKYLDENKKLIMAILENQNLGKLAECAQYQALLQKNLMYLAAIADAQPQPP

SEQ ID NO: 100 - protein, 59 aa, Artificial Sequence
  Synthetic construct
TTVTTDLIQQYLDENKQLILAILDNQNNGKVEECARNQAKLQQNLMYLAAIADSQPPQT

SEQ ID NO: 101 - protein, 59 aa, Artificial Sequence
  Synthetic construct
AGITTEQIQKYLDENKQLILAILENQNLGKLAECAQYQAQLQKNLLYLAAIADAQPPQN

SEQ ID NO: 102 - protein, 59 aa, Artificial Sequence
  Synthetic construct
AGLSTEQIQKYLDENKQLILAILENQNLGKLAECAQYQAQLQKNLLYLAAIADTQPQTT

SEQ ID NO: 103 - protein, 58 aa, Artificial Sequence
  Synthetic construct
NVTTDHIQQYLDENKSLILKIVESQNSGKLTECAENQARLQRNLMYLAAIADSQPQPP

SEQ ID NO: 104 - DNA, 20 bp, Artificial Sequence
  Synthetic construct
cctccgactccaagtattgc

SEQ ID NO: 105 - DNA, 20 bp, Artificial Sequence
  Synthetic construct
atcatcaggttggacgggta

SEQ ID NO: 106 - DNA, 52 bp, Artificial Sequence
  Synthetic construct
ggggacaagtttgtacaaaaaagctgccaccatggcgatgccgtatgcctct

SEQ ID NO: 107 - DNA, 51 bp, Artificial Sequence
  Synthetic construct
ggggaccactttgtacaagaaagctgaacggtacatytcgccggcgaacag

SEQ ID NO: 108 - DNA, 52 bp, Artificial Sequence
  Synthetic construct
ggggacaagtttgtacaaaaaagctgccaccatgcagcagcaacacctgatg

SEQ ID NO: 109 - DNA, 48 bp, Artificial Sequence
  Synthetic construct
ggggaccactttgtacaagaaagctgaacggcttccttcctcctcggt

SEQ ID NO: 110 - DNA, 52 bp, Artificial Sequence; identical to SEQ ID NO: 106
  Synthetic construct

SEQ ID NO: 111 - DNA, 33 bp, Artificial Sequence
  Synthetic construct
ggcagcggccgcgtacatytcgccggcgaacag

SEQ ID NO: 112 - DNA, 33 bp, Artificial Sequence
  Synthetic construct
gcggccgctgccatgcagcagcaacacctgatg

SEQ ID NO: 113 - DNA, 51 bp, Artificial Sequence
  Synthetic construct
ggggaccactttgtacaagaaagctgaacgctagcttccttcctcctcggt

SEQ ID NO: 114 - DNA, 32 bp, Artificial Sequence
  Synthetic construct
aagctgccgcggccatgcagcagcaacacctg

SEQ ID NO: 115 - DNA, 33 bp, Artificial Sequence
  Synthetic construct
tacgcggccgctgccgaagctcgaaaaacaaag

SEQ ID NO: 116 - DNA, 33 bp, Artificial Sequence
  Synthetic construct
catggccgcggcagctttttgatgaaacagaag

SEQ ID NO: 117 - DNA, 28 bp, Artificial Sequence
  Synthetic construct
tctagaaaaccggtcgaaacgcagctcg

SEQ ID NO: 118 - DNA, 27 bp, Artificial Sequence
  Synthetic construct
tcgaccggttttctagaacggttgcgg

SEQ ID NO: 119 - DNA, 24 bp, Artificial Sequence
  Synthetic construct
gccactcagcaagctttgcagcgt

SEQ ID NO: 120 - DNA, 35 bp, Artificial Sequence
  Synthetic construct
tcacgctgcaaagctctaattcccgatctagtaac

SEQ ID NO: 121 - DNA, 50 bp, Artificial Sequence
  Synthetic construct
ggggacaagtttgtacaaaaaagctgccaccatgaagcaaagctttgtgg

SEQ ID NO: 122 - DNA, 27 bp, Artificial Sequence
  Synthetic construct
tcgaccggttttctagaacggttgcgg

SEQ ID NO: 123 - DNA, 28 bp, Artificial Sequence
  Synthetic construct
tctagaaaaccggtcgaatcacaaacta

SEQ ID NO: 124 - DNA, 49 bp, Artificial Sequence
  Synthetic construct
ggggaccactttgtacaagaaagctgaacgtcaattcccatcttcagca

SEQ ID NO: 125 - protein, 82 aa, Artificial Sequence
  Synthetic construct
PFTATQWQELEHQALIYKYMASGVPIPSDLLLPLRRSFDPEPGRCRRTDGKKWRCSKEAYPDSKYCEKHMHRGKNRSRKPVE

SEQ ID NO: 126 - protein, 82 aa, Artificial Sequence
  Synthetic construct
LFTASQWRELEHQALIYKYMAAGSQVPHELVLPLRHRDDPEPGRCRRTDGKKWRCSREAYGESKYCDRHMHRGKNRSRKPVE

SEQ ID NO: 127 - protein, 82 aa, Artificial Sequence
  Synthetic construct
PFTAAQYEELEHQALIYKYLVAGVSVPPDLVLPIRRGIDPEPGRCRRTDGKKWRCAKEAASDSKYCERHMHRGRNRSRKPVE

SEQ ID NO: 128 - protein, 82 aa, Artificial Sequence
  Synthetic construct
TFTAAQYEELEQQALIYKYLVAGVPVPPDLLLPIRRGFDPEPGRCRRTDGKKWRCSKEAAQDSKYCERHMHRGRNRSRKPVE

SEQ ID NO: 129 - protein, 82 aa, Artificial Sequence
  Synthetic construct
VFTPAQWAELEQQALIYKYLMAGVPVPPDLLLPIRPHPDPEPWRCRRTDGKKWRCSKEAHPDSKYCERHMHRGRNRSRKPVE

SEQ ID NO: 130 - protein, 82 aa, Artificial Sequence
  Synthetic construct
PFTPSQWMELEHQALIYKYLNAKAPIPSGLLISISKSFDPEPGRCRRTDGKKWRCSKEAMAEHKYCERHINRNRHRSRKPVE

SEQ ID NO: 131 - protein, 82 aa, Artificial Sequence
  Synthetic construct
PFTPSQWMELEHQALIYKYLAANIAVPHSLLVPIRRSVDLEPGRCRRTDGKKWRCSRDAVADQKYCERHMNRGRHRSRKHVE

SEQ ID NO: 132 - protein, 82 aa, Artificial Sequence
  Synthetic construct
ALTFMQQQELEHQVLIYRYFAAGAPVPVHLVLPIWKSVEPEPGRCRRTDGKKWRCSRDVVQGHKYCERHVHRGRGRSRKPVE

SEQ ID NO: 133 - protein, 205 aa, Oryza sativa
MQQQMAMPAGAAAAAVPPAAGITTEQIQKYLDENKQLILAILENQNLGKLAECAQYQAQLQKNLLYLAAIADAQPPQNPGSRPQMMQPGATPGAGHYMSQVPMFPPRTPLTPQQMQEQQQQQLQQQQAQALAFPGQMLMRPGTVNGMQSIPVADPARAADLQTAAPGSVDGRGNKQDATSEPSGTESHKSAGADNDAGGDIAEKS

SEQ ID NO: 134 - protein, 185 aa, Oryza sativa
MQQQPMPMPAQAPPTAGITTEQIQKYLDENKQLILAILENQNLGKLAECAQYQAQLQKNLLYLAAIADTQPQTTISRPQMVPHGASPGLGGQYMSQVPMFPPRTPLTPQQMQEQQLQQQQAQLLSFGGQMVMRPGVVNGIPQLLQGEMHRGADHQNAGGATSEPSESHRSTGTENDGGSDFGDQS

SEQ ID NO: 135 - protein, 227 aa, Oryza sativa
MQQQHLMQMNQGMMGGYASPTTVTTDLIQQYLDENKQLILAILDNQNNGKVEECARNQAKLQHNLMYLAAIADSQPPQTAAMSQYPSNLMMQSGARYMPQQSAQMMAPQSLMAARSSMMYAQPALSPLQQQQQQQAAAAHGQLGMGSGGTTSGFSILHGEASMGGGGGGGGAGNSMMNAGVFSDFGRGGGGGGKEGSTSLSVDVRGANSGAQSGDGEYLKGTEEEGS

SEQ ID NO: 136 - protein, 59 aa, Artificial Sequence; identical to SEQ ID NO: 101
  Synthetic construct

SEQ ID NO: 137 - protein, 59 aa, Artificial Sequence
  Synthetic construct
TTVTTDLIQQYLDENKQLILAILDNQNNGKVEECARNQAKLQHNLMYLAAIADSQPPQT

SEQ ID NO: 138 - protein, 360 aa, Capsicum annuum
MSGTSTSVVGGGGEGGMGYGYGYRPPFTAVQWQELEHQAMIYKYLVAGLPVPPDLVVPIRRSFEAISARFFHHPSLGYCSYYGKKFDPEPGRCRRTDGKKWRCSKDAYPDSKYCERHMHRGRNRSRKPVESQSTSQSLSTSMSQITTGSSNTRGSFQTSGSGSFQNMPLYSVANSGALNYGSTATKLQKEPVSCGIDNKDYRYLHGIAPDADEHNLSSEASATVRSFGMGTNTDGTWVFPSQVSSSPMARQKNDSQMLGSSTEMHLPNLLEPMIDSTISKRRHQHCFFGSDIDSPGTVKEEQQHSMRPFFNEWPTAKESWSNLDDEGSNKNNFSTTQLSISIPIAPSNFSSRSACSPNDA

SEQ ID NO: 139 - protein, 215 aa, Capsicum annuum
MQQHLMQMQPMMAAYYPTNVTTDHIQQYLDENKSLILKIVESQNSGKLGECAENQARLQRNLMYLAAIADSQPQPSSMHSQFSSGGMMQPGTHNYLQQQHQQAQQIATQSLIAARSSSMLYGQQQQQQQHQLSPFQQGLHGSQLGMSSGSGGSTGFHMLQSESSPHGGGGGSGGFPHDFGRANKQDIGSTMSAEGRGRSSGGDGGENLYLKASED

SEQ ID NO: 140 - DNA, 1740 bp, Artificial Sequence
  Synthetic construct
atgagtggcacctcaacatcagtggtcgggggtgggggtgaggggggaatggggtatgggtatggttaccggccaccgtttactgcagtgcaatggcaggagctagaacatcaagcaatgatatataagtacttagtggcaggtcttcctgtgccacctgatcttgttgttcctattcgtcgtagctttgaagctatctcagctaggttctttcatcatcccagcttgggctattgttcctattatggaaagaagtttgatcctgagcctggaaggtgtagaaggactgatggaaagaagtggagatgctcgaaagatgcatatcccgactccaaatattgcgagcgtcacatgcatcgaggccgcaaccgttcaagaaagcctgtggaatctcaatctacttcccaatccttgtcgactagtatgtcacaaattacaactgggagcagtaataccagaggaagtttccaaactagcggcagcggaagcttccaaaacatgccattgtattctgttgctaattcaggagcgctgaattatggaagcactgcaacaaagttgcagaaggagcctgtctcctgtggaatagataacaaggactataggtatctccatggaattgctccagatgctgatgagcataatttatcttcagaagcttctgcaactgtcagaagttttgggatggggaccaacacagacggtacctgggtatttccttctcaagtttcttcaagccccatggcgagacaaaagaatgattcccagatgctgggtagctcaacagagatgcatctacctaatctacttgagcctatgattgactcaacaatttcaaagcgacgacaccagcattgcttctttggcagtgacattgattcacctggaacagtaaaggaggagcagcagcattcgatgcgccctttctttaacgaatggcccactgctaaagaatcgtggtccaaccttgacgatgagggatccaacaaaaacaatttctccactacacaactatccatatccattccaatcgctccttccaacttctcttcaaggagtgcttgctccccaaatgatgctgcggccgctgccatgcagcagcacctgatgcagatgcagcccatgatggcagcttactatccaacaaacgtcactactgaccatattcaacagtatttggatgagaacaaatcactcattctgaagattgttgagagccagaactccgggaaactcggcgaatgtgcagagaaccaggctaggcttcagaggaatctgatgtaccttgctgctatcgctgattcacaacctcaaccttctagcatgcattctcagttctcttctggtggcatgatgcagccagggacacacaattacctgcagcagcagcaccaacaagcccaacaaatagcaacacaatcactcatagctgcaagatcctcttcaatgctctatggacaacaacaacaacaacagcagcatcaattatcgccatttcaacaaggcttgcatggtagccaacttggcatgagctccggcagcggtggaagcactggttttcacatgcttcaaagtgaatcatcacctcatggtggtggtggtggaagtggtggtttccctcatgacttcggccgtgcaaataagcaagacattgggagtactatgtctgctgaagggcgcggcagaagctcaggtggtgatggtggtgagaatctttatcttaaagcttctgaggattga